SEQUENCE LISTING (373 sequences)

SEQ ID NO: 1 - DNA, 357 bp, Artificial Sequence
  synthetic
caggtgcagctggtggagtctgggggaggcgtggtccagcctggaaggtccctgagactctcctgtgtagcgtctggattcaccttcagtagttatggcattcactgggtccgccaggctccaggcaaggggctggagtgggtggcagttatatgggatgatggaaataatataaactattcagactccgtgaagggccgattcatcatctccagagacaattccaggaagacagtgtatctgcaaatgaacagcctgagaggcgaggacacggctgtttattactgtgcgagagatgcccccatagcaccagtccctgactattggggccagggaaccctggtcaccgtctcctca

SEQ ID NO: 2 - protein, 119 aa, Artificial Sequence
  synthetic
QVQLVESGGGVVQPGRSLRLSCVASGFTFSSYGIHWVRQAPGKGLEWVAVIWDDGNNINYSDSVKGRFIISRDNSRKTVYLQMNSLRGEDTAVYYCARDAPIAPVPDYWGQGTLVTVSS

SEQ ID NO: 3 - DNA, 24 bp, Artificial Sequence
  synthetic
ggattcaccttcagtagttatggc

SEQ ID NO: 4 - protein, 8 aa, Artificial Sequence
  synthetic
GFTFSSYG

SEQ ID NO: 5 - DNA, 24 bp, Artificial Sequence
  synthetic
atatgggatgatggaaataatata

SEQ ID NO: 6 - protein, 8 aa, Artificial Sequence
  synthetic
IWDDGNNI

SEQ ID NO: 7 - DNA, 36 bp, Artificial Sequence
  synthetic
gcgagagatgcccccatagcaccagtccctgactat

SEQ ID NO: 8 - protein, 12 aa, Artificial Sequence
  synthetic
ARDAPIAPVPDY

SEQ ID NO: 9 - DNA, 321 bp, Artificial Sequence
  synthetic
gacatccagatgacccagtctccttccaccctgtctgcatctgtaggagacagagtcaccatcacttgccgggccagtcagagtattagtagttggttggcctggtatcagcagaaaccagggaaagcccctaagctcctgatctataaggcgtctagtttagacactggggtcccatcaaggttcagcggcagtggatctgggacagagttcactctcaccatcagcagcctgcagcctgatgattttgcaacttattactgccaacagtataatacttattcgtacacttttggcctggggaccaaactggagatcaaa

SEQ ID NO: 10 - protein, 107 aa, Artificial Sequence
  synthetic
DIQMTQSPSTLSASVGDRVTITCRASQSISSWLAWYQQKPGKAPKLLIYKASSLDTGVPSRFSGSGSGTEFTLTISSLQPDDFATYYCQQYNTYSYTFGLGTKLEIK

SEQ ID NO: 11 - DNA, 18 bp, Artificial Sequence
  synthetic
cagagtattagtagttgg

SEQ ID NO: 12 - protein, 6 aa, Artificial Sequence
  synthetic
QSISSW

SEQ ID NO: 13 - DNA, 9 bp, Artificial Sequence
  synthetic
aaggcgtct

SEQ ID NO: 14 - protein, 3 aa, Artificial Sequence
  synthetic
KAS

SEQ ID NO: 15 - DNA, 27 bp, Artificial Sequence
  synthetic
caacagtataatacttattcgtacact

SEQ ID NO: 16 - protein, 9 aa, Artificial Sequence
  synthetic
QQYNTYSYT

SEQ ID NO: 17 - DNA, 357 bp, Artificial Sequence
  synthetic
caggtgcaactggtggagtctgggggaggcttggtcaagcctggagggtccctgagactctcctgtgcagcttctggattcaccttcagtgactactacatgagctggatccgccaggctccagggaaggggctggagtgggtttcatatattagcagtagtggtaataccataaaatatgcagactctatgaagggccgattcaccatctccagggacaacgccaagaaatcactgtttgtggaaatgaacagcctgagagccgaggacacggccgtgtattactgtgcgaggtataaaagttcgtccgactactttgaccactggggccagggaaccctggtcaccgtctcctca

SEQ ID NO: 18 - protein, 119 aa, Artificial Sequence
  synthetic
QVQLVESGGGLVKPGGSLRLSCAASGFTFSDYYMSWIRQAPGKGLEWVSYISSSGNTIKYADSMKGRFTISRDNAKKSLFVEMNSLRAEDTAVYYCARYKSSSDYFDHWGQGTLVTVSS

SEQ ID NO: 19 - DNA, 24 bp, Artificial Sequence
  synthetic
ggattcaccttcagtgactactac

SEQ ID NO: 20 - protein, 8 aa, Artificial Sequence
  synthetic
GFTFSDYY

SEQ ID NO: 21 - DNA, 24 bp, Artificial Sequence
  synthetic
attagcagtagtggtaataccata

SEQ ID NO: 22 - protein, 8 aa, Artificial Sequence
  synthetic
ISSSGNTI

SEQ ID NO: 23 - DNA, 36 bp, Artificial Sequence
  synthetic
gcgaggtataaaagttcgtccgactactttgaccac

SEQ ID NO: 24 - protein, 12 aa, Artificial Sequence
  synthetic
ARYKSSSDYFDH

SEQ ID NO: 25 - DNA, 321 bp, Artificial Sequence
  synthetic
gaaattgtgttgacacagtctccagccaccctgtctttgtctccaggggaaagagccaccctctcctgcagggccagtcagagtgttaggagttacttagcctggtaccaacagaaacctggccaggctcccaggctcctcatctatgatgcatccaacagggccactgccatcccagccaggttcagtggcagtgggtctgggacagacttcactctcaccatcagcagcctagagcctgaagatttagcagtttattactgtcagcagtctggcaactggccgctcactttcggcggagggaccaaggtggagatcaaa

SEQ ID NO: 26 - protein, 107 aa, Artificial Sequence
  synthetic
EIVLTQSPATLSLSPGERATLSCRASQSVRSYLAWYQQKPGQAPRLLIYDASNRATAIPARFSGSGSGTDFTLTISSLEPEDLAVYYCQQSGNWPLTFGGGTKVEIK

SEQ ID NO: 27 - DNA, 18 bp, Artificial Sequence
  synthetic
cagagtgttaggagttac

SEQ ID NO: 28 - protein, 6 aa, Artificial Sequence
  synthetic
QSVRSY

SEQ ID NO: 29 - DNA, 9 bp, Artificial Sequence
  synthetic
gatgcatcc

SEQ ID NO: 30 - protein, 3 aa, Artificial Sequence
  synthetic
DAS

SEQ ID NO: 31 - DNA, 27 bp, Artificial Sequence
  synthetic
cagcagtctggcaactggccgctcact

SEQ ID NO: 32 - protein, 9 aa, Artificial Sequence
  synthetic
QQSGNWPLT

SEQ ID NO: 33 - DNA, 357 bp, Artificial Sequence
  synthetic
caggtgcagctggtggagtctgggggaggcgtggtccagcctgggaggtccttgagactctcctgtggagcgtctggattcaccttcagtacttatggcatgcactgggtccgccaggctccaggcaaggggctggagtgggtggcagttatctgggatgatggaaataataaatattatgcagactccgtgaagggccgattcaccatctccagagacaattcgaagaacacgctgtatctgcagatgaacagcctgagagccgaggacacggctgtttattactgtgcgagagattcagaggtcgccccagttggggactactggggccagggcaccctggtcaccgtctcctca

SEQ ID NO: 34 - protein, 119 aa, Artificial Sequence
  synthetic
QVQLVESGGGVVQPGRSLRLSCGASGFTFSTYGMHWVRQAPGKGLEWVAVIWDDGNNKYYADSVKGRFTISRDNSKNTLYLQMNSLRAEDTAVYYCARDSEVAPVGDYWGQGTLVTVSS

SEQ ID NO: 35 - DNA, 24 bp, Artificial Sequence
  synthetic
ggattcaccttcagtacttatggc

SEQ ID NO: 36 - protein, 8 aa, Artificial Sequence
  synthetic
GFTFSTYG

SEQ ID NO: 37 - DNA, 24 bp, Artificial Sequence
  synthetic
atctgggatgatggaaataataaa

SEQ ID NO: 38 - protein, 8 aa, Artificial Sequence
  synthetic
IWDDGNNK

SEQ ID NO: 39 - DNA, 36 bp, Artificial Sequence
  synthetic
gcgagagattcagaggtcgccccagttggggactac

SEQ ID NO: 40 - protein, 12 aa, Artificial Sequence
  synthetic
ARDSEVAPVGDY

SEQ ID NO: 41 - DNA, 321 bp, Artificial Sequence
  synthetic
gacatccagatgacccagtctccttccaccctgtctgcatctgtaggagacagagtcactatcatttgccgggccagtcagagtattaacaggtggttggcctggtatcagcagaaaccagggaaggcccctaaactcctgatctataaggcgtctagtttagaaagtggggtcccatcaaggttcagcggcagtggatctgggacagaattcactctcaccatcagcagcctgcagcctgatgattttgcagcttattactgccaacagtataatgattattcgtacacttttggccaggggaccaagctggagatcaaa

SEQ ID NO: 42 - protein, 107 aa, Artificial Sequence
  synthetic
DIQMTQSPSTLSASVGDRVTIICRASQSINRWLAWYQQKPGKAPKLLIYKASSLESGVPSRFSGSGSGTEFTLTISSLQPDDFAAYYCQQYNDYSYTFGQGTKLEIK

SEQ ID NO: 43 - DNA, 18 bp, Artificial Sequence
  synthetic
cagagtattaacaggtgg

SEQ ID NO: 44 - protein, 6 aa, Artificial Sequence
  synthetic
QSINRW

SEQ ID NO: 45 - DNA, 9 bp, Artificial Sequence
  synthetic
aaggcgtct

SEQ ID NO: 46 - protein, 3 aa, Artificial Sequence
  synthetic
KAS

SEQ ID NO: 47 - DNA, 27 bp, Artificial Sequence
  synthetic
caacagtataatgattattcgtacact

SEQ ID NO: 48 - protein, 9 aa, Artificial Sequence
  synthetic
QQYNDYSYT

SEQ ID NO: 49 - DNA, 366 bp, Artificial Sequence
  synthetic
gaggtgcagctggtggagtctgggggagacttggtccagcctggagggtccctgagactctcctgtgcagcctctggattcaccttcagtgaccactatatggactgggtccgccaggctccagggaaggggctggactggattggccgtattagaaacaaagctaacgcttataacacagaatacgccgcgtctgtgagaggcagattcaccatctcaagagatgattcacagaatttactgtatctgcaaatgaacagcctgaaaaccgatgacacggccgtatattattgtgttagagtctggaactacgcctacttcgctatggacgtctggggccaagggaccacggtcaccgtctcctca

SEQ ID NO: 50 - protein, 122 aa, Artificial Sequence
  synthetic
EVQLVESGGDLVQPGGSLRLSCAASGFTFSDHYMDWVRQAPGKGLDWIGRIRNKANAYNTEYAASVRGRFTISRDDSQNLLYLQMNSLKTDDTAVYYCVRVWNYAYFAMDVWGQGTTVTVSS

SEQ ID NO: 51 - DNA, 24 bp, Artificial Sequence
  synthetic
ggattcaccttcagtgaccactat

SEQ ID NO: 52 - protein, 8 aa, Artificial Sequence
  synthetic
GFTFSDHY

SEQ ID NO: 53 - DNA, 30 bp, Artificial Sequence
  synthetic
attagaaacaaagctaacgcttataacaca

SEQ ID NO: 54 - protein, 10 aa, Artificial Sequence
  synthetic
IRNKANAYNT

SEQ ID NO: 55 - DNA, 39 bp, Artificial Sequence
  synthetic
gttagagtctggaactacgcctacttcgctatggacgtc

SEQ ID NO: 56 - protein, 13 aa, Artificial Sequence
  synthetic
VRVWNYAYFAMDV

SEQ ID NO: 57 - DNA, 321 bp, Artificial Sequence
  synthetic
gacatccagatgacccagtctccatcctccctatctgcatctgtgggagacagagtcaccatcacttgccggtcaagtcagaacattggaatctttttaaactggtatcaacaaaaaccaggggaagcccctaacctcctgatctccgctgcatccagtttacacagtggggtcccttcaaggttcagtggcagtgggtctgggacagatttcactctcaccatcggcagtctgcagcctgaagattttgcgacttactactgtcaacagacgtacaataccatattcactttcggccctgggaccaaagtggatatcaaa

SEQ ID NO: 58 - protein, 107 aa, Artificial Sequence
  synthetic
DIQMTQSPSSLSASVGDRVTITCRSSQNIGIFLNWYQQKPGEAPNLLISAASSLHSGVPSRFSGSGSGTDFTLTIGSLQPEDFATYYCQQTYNTIFTFGPGTKVDIK

SEQ ID NO: 59 - DNA, 18 bp, Artificial Sequence
  synthetic
cagaacattggaatcttt

SEQ ID NO: 60 - protein, 6 aa, Artificial Sequence
  synthetic
QNIGIF

SEQ ID NO: 61 - DNA, 9 bp, Artificial Sequence
  synthetic
gctgcatcc

SEQ ID NO: 62 - protein, 3 aa, Artificial Sequence
  synthetic
AAS

SEQ ID NO: 63 - DNA, 27 bp, Artificial Sequence
  synthetic
caacagacgtacaataccatattcact

SEQ ID NO: 64 - protein, 9 aa, Artificial Sequence
  synthetic
QQTYNTIFT

SEQ ID NO: 65 - DNA, 363 bp, Artificial Sequence
  synthetic
gaggtgcagctggtggagtctgggggagacttggtacagcctggggggtccctgagactctcctgtgcagcctctggattcacctttagcagctatgccatgaactgggtccgccagggtccagggaagggactggagtgggtctcagctattagtggtcgtggtgatagtacatactacgcagactccgtgaagggccggctcaccatctccagagacaattccaagaacacgctgtatctgcaaatgaacagcctgagagccgaggacacggccgtatattactgtgtgaaagagggggagcaactcgtctactggtacttcgatctctggggccgtggcaccctggtcaccgtctcctca

SEQ ID NO: 66 - protein, 121 aa, Artificial Sequence
  synthetic
EVQLVESGGDLVQPGGSLRLSCAASGFTFSSYAMNWVRQGPGKGLEWVSAISGRGDSTYYADSVKGRLTISRDNSKNTLYLQMNSLRAEDTAVYYCVKEGEQLVYWYFDLWGRGTLVTVSS

SEQ ID NO: 67 - DNA, 24 bp, Artificial Sequence
  synthetic
ggattcacctttagcagctatgcc

SEQ ID NO: 68 - protein, 8 aa, Artificial Sequence
  synthetic
GFTFSSYA

SEQ ID NO: 69 - DNA, 24 bp, Artificial Sequence
  synthetic
attagtggtcgtggtgatagtaca

SEQ ID NO: 70 - protein, 8 aa, Artificial Sequence
  synthetic
ISGRGDST

SEQ ID NO: 71 - DNA, 42 bp, Artificial Sequence
  synthetic
gtgaaagagggggagcaactcgtctactggtacttcgatctc

SEQ ID NO: 72 - protein, 14 aa, Artificial Sequence
  synthetic
VKEGEQLVYWYFDL

SEQ ID NO: 73 - DNA, 321 bp, Artificial Sequence
  synthetic
gacatccagatgacccagtctccatcctccctgtctgcatctgtaggagacagagtcaccatcacttgccgggcaagtcagaccattagcaactttttacattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagatttttcaacttacttctgtcaacagagttacactaccccgctcactttcggcggagggaccaaggtggagatcaaa

SEQ ID NO: 74 - protein, 107 aa, Artificial Sequence
  synthetic
DIQMTQSPSSLSASVGDRVTITCRASQTISNFLHWYQQKPGKAPKLLIYAASSLQSGVPSRFSGSGSGTDFTLTISSLQPEDFSTYFCQQSYTTPLTFGGGTKVEIK

SEQ ID NO: 75 - DNA, 18 bp, Artificial Sequence
  synthetic
cagaccattagcaacttt

SEQ ID NO: 76 - protein, 6 aa, Artificial Sequence
  synthetic
QTISNF

SEQ ID NO: 77 - DNA, 9 bp, Artificial Sequence
  synthetic
gctgcatcc

SEQ ID NO: 78 - protein, 3 aa, Artificial Sequence
  synthetic
AAS

SEQ ID NO: 79 - DNA, 27 bp, Artificial Sequence
  synthetic
caacagagttacactaccccgctcact

SEQ ID NO: 80 - protein, 9 aa, Artificial Sequence
  synthetic
QQSYTTPLT

SEQ ID NO: 81 - DNA, 363 bp, Artificial Sequence
  synthetic
gaggtgcagctggtggagtctgggggaggcttggtgaggtcgggggggtccctgagactctcctgtgcagcctctggattcacctttaacagatatgccatgacctgggtccgccaggctccagggaaggggctggagtgggtctcagctataagtggtagtggtagcagcacatactacacagactccgtgaaaggccggttcaccatctccagagacaattccaagaattcggtggatctgcaaatgcacagcctgagagtcgaagacacggccatatattattgtgcgagagggactacagtcactacggggtacggtatggacgtctggggccaagggaccacggtcaccgtctcctca

SEQ ID NO: 82 - protein, 121 aa, Artificial Sequence
  synthetic
EVQLVESGGGLVRSGGSLRLSCAASGFTFNRYAMTWVRQAPGKGLEWVSAISGSGSSTYYTDSVKGRFTISRDNSKNSVDLQMHSLRVEDTAIYYCARGTTVTTGYGMDVWGQGTTVTVSS

SEQ ID NO: 83 - DNA, 24 bp, Artificial Sequence
  synthetic
ggattcacctttaacagatatgcc

SEQ ID NO: 84 - protein, 8 aa, Artificial Sequence
  synthetic
GFTFNRYA

SEQ ID NO: 85 - DNA, 24 bp, Artificial Sequence
  synthetic
ataagtggtagtggtagcagcaca

SEQ ID NO: 86 - protein, 8 aa, Artificial Sequence
  synthetic
ISGSGSST

SEQ ID NO: 87 - DNA, 42 bp, Artificial Sequence
  synthetic
gcgagagggactacagtcactacggggtacggtatggacgtc

SEQ ID NO: 88 - protein, 14 aa, Artificial Sequence
  synthetic
ARGTTVTTGYGMDV

SEQ ID NO: 89 - DNA, 321 bp, Artificial Sequence
  synthetic
gacatccagatgacccagtctccatcctccctgtctgcatctgtaggagacagagtcaccttcacttgccaggcgagtcaggacattaccaattctttaaattggtatcaacagaaacctgggagagcccctaagctcctgatctacgatgcatcgtatttgaaggcaggggtcccatcaagattcagtggaagtggatctgggacagattttactttcaccatcagcagcctgcagcctgaagatattgcaacatattactgtcaacaatatgatgatctcccatacacttttggccaggggaccaagctggagatcaaa

SEQ ID NO: 90 - protein, 107 aa, Artificial Sequence
  synthetic
DIQMTQSPSSLSASVGDRVTFTCQASQDITNSLNWYQQKPGRAPKLLIYDASYLKAGVPSRFSGSGSGTDFTFTISSLQPEDIATYYCQQYDDLPYTFGQGTKLEIK

SEQ ID NO: 91 - DNA, 18 bp, Artificial Sequence
  synthetic
caggacattaccaattct

SEQ ID NO: 92 - protein, 6 aa, Artificial Sequence
  synthetic
QDITNS

SEQ ID NO: 93 - DNA, 9 bp, Artificial Sequence
  synthetic
gatgcatcg

SEQ ID NO: 94 - protein, 3 aa, Artificial Sequence
  synthetic
DAS

SEQ ID NO: 95 - DNA, 27 bp, Artificial Sequence
  synthetic
caacaatatgatgatctcccatacact

SEQ ID NO: 96 - protein, 9 aa, Artificial Sequence
  synthetic
QQYDDLPYT

SEQ ID NO: 97 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatatttgactactggggccagggaaccctggtcaccgtctcctca

SEQ ID NO: 98 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 99 - DNA, 24 bp, Artificial Sequence
  synthetic
ggtgactccgtcagtagttcctac

SEQ ID NO: 100 - protein, 8 aa, Artificial Sequence
  synthetic
GDSVSSSY

SEQ ID NO: 101 - DNA, 21 bp, Artificial Sequence
  synthetic
atctattacagtgggagttcc

SEQ ID NO: 102 - protein, 7 aa, Artificial Sequence
  synthetic
IYYSGSS

SEQ ID NO: 103 - DNA, 42 bp, Artificial Sequence
  synthetic
gcgagagaagggaacgtggatacaactatgatatttgactac

SEQ ID NO: 104 - protein, 14 aa, Artificial Sequence
  synthetic
AREGNVDTTMIFDY

SEQ ID NO: 105 - DNA, 321 bp, Artificial Sequence
  synthetic
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggacgttcggccaagggaccaaggtggaaatcaaa

SEQ ID NO: 106 - protein, 107 aa, Artificial Sequence
  synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 107 - DNA, 18 bp, Artificial Sequence
  synthetic
cagggcattagaaatgat

SEQ ID NO: 108 - protein, 6 aa, Artificial Sequence
  synthetic
QGIRND

SEQ ID NO: 109 - DNA, 9 bp, Artificial Sequence
  synthetic
gctgcatcc

SEQ ID NO: 110 - protein, 3 aa, Artificial Sequence
  synthetic
AAS

SEQ ID NO: 111 - DNA, 27 bp, Artificial Sequence
  synthetic
ctacaagatttcaattacccgtggacg

SEQ ID NO: 112 - protein, 9 aa, Artificial Sequence
  synthetic
LQDFNYPWT

SEQ ID NO: 113 - DNA, 321 bp, Artificial Sequence
  synthetic
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtcatcaagatttcaattacccgtggacgttcggccaagggaccaaggtggaaatcaaa

SEQ ID NO: 114 - protein, 107 aa, Artificial Sequence
  synthetic
AIQMTQSPSSLSASVGDRVTITCRASQGIRNDLGWYQQKPGKAPKLLIYAASSLQSGVPSRFAGRGSGTDFTLTISSLQPEDFATYYCHQDFNYPWTFGQGTKVEIK

SEQ ID NO: 115 - DNA, 18 bp, Artificial Sequence
  synthetic
cagggcattagaaatgat

SEQ ID NO: 116 - protein, 6 aa, Artificial Sequence
  synthetic
QGIRND

SEQ ID NO: 117 - DNA, 9 bp, Artificial Sequence
  synthetic
gctgcatcc

SEQ ID NO: 118 - protein, 3 aa, Artificial Sequence
  synthetic
AAS

SEQ ID NO: 119 - DNA, 27 bp, Artificial Sequence
  synthetic
catcaagatttcaattacccgtggacg

SEQ ID NO: 120 - protein, 9 aa, Artificial Sequence
  synthetic
HQDFNYPWT

SEQ ID NO: 121 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaacataacgtggatacaactatgatatttgactactggggccagggaaccctggtcaccgtctcctca

SEQ ID NO: 122 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREHNVDTTMIFDYWGQGTLVTVSS

SEQ ID NO: 123 - DNA, 24 bp, Artificial Sequence
  synthetic
ggtgactccgtcagtagttcctac

SEQ ID NO: 124 - protein, 8 aa, Artificial Sequence
  synthetic
GDSVSSSY

SEQ ID NO: 125 - DNA, 21 bp, Artificial Sequence
  synthetic
atctattacagtgggagttcc

SEQ ID NO: 126 - protein, 7 aa, Artificial Sequence
  synthetic
IYYSGSS

SEQ ID NO: 127 - DNA, 42 bp, Artificial Sequence
  synthetic
gcgagagaacataacgtggatacaactatgatatttgactac

SEQ ID NO: 128 - protein, 14 aa, Artificial Sequence
  synthetic
AREHNVDTTMIFDY

SEQ ID NO: 129 - DNA, 321 bp, Artificial Sequence
  synthetic
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggcacttcggccaagggaccaaggtggaaatcaaa

SEQ ID NO: 130 - protein, 107 aa, Artificial Sequence
  synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWHFGQGTKVEIK

SEQ ID NO: 131 - DNA, 18 bp, Artificial Sequence
  synthetic
cagggcattagaaatgat

SEQ ID NO: 132 - protein, 6 aa, Artificial Sequence
  synthetic
QGIRND

SEQ ID NO: 133 - DNA, 9 bp, Artificial Sequence
  synthetic
gctgcatcc

SEQ ID NO: 134 - protein, 3 aa, Artificial Sequence
  synthetic
AAS

SEQ ID NO: 135 - DNA, 27 bp, Artificial Sequence
  synthetic
ctacaagatttcaattacccgtggcac

SEQ ID NO: 136 - protein, 9 aa, Artificial Sequence
  synthetic
LQDFNYPWH

SEQ ID NO: 137 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatacatgactactggggccagggaaccctggtcaccgtctcctca

SEQ ID NO: 138 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIHDYWGQGTLVTVSS

SEQ ID NO: 139 - DNA, 24 bp, Artificial Sequence
  synthetic
ggtgactccgtcagtagttcctac

SEQ ID NO: 140 - protein, 8 aa, Artificial Sequence
  synthetic
GDSVSSSY

SEQ ID NO: 141 - DNA, 21 bp, Artificial Sequence
  synthetic
atctattacagtgggagttcc

SEQ ID NO: 142 - protein, 7 aa, Artificial Sequence
  synthetic
IYYSGSS

SEQ ID NO: 143 - DNA, 42 bp, Artificial Sequence
  synthetic
gcgagagaagggaacgtggatacaactatgatacatgactac

SEQ ID NO: 144 - protein, 14 aa, Artificial Sequence
  synthetic
AREGNVDTTMIHDY

SEQ ID NO: 145 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatcacactatgatatttgactactggggccagggaaccctggtcaccgtctcctca

SEQ ID NO: 146 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDHTMIFDYWGQGTLVTVSS

SEQ ID NO: 147 - DNA, 24 bp, Artificial Sequence
  synthetic
ggtgactccgtcagtagttcctac

SEQ ID NO: 148 - protein, 8 aa, Artificial Sequence
  synthetic
GDSVSSSY

SEQ ID NO: 149 - DNA, 21 bp, Artificial Sequence
  synthetic
atctattacagtgggagttcc

SEQ ID NO: 150 - protein, 7 aa, Artificial Sequence
  synthetic
IYYSGSS

SEQ ID NO: 151 - DNA, 42 bp, Artificial Sequence
  synthetic
gcgagagaagggaacgtggatcacactatgatatttgactac

SEQ ID NO: 152 - protein, 14 aa, Artificial Sequence
  synthetic
AREGNVDHTMIFDY

SEQ ID NO: 153 - DNA, 360 bp, Artificial Sequence
  synthetic
caggtgcagctggtggagtctgggggaggcttggtcaagcctggagggtccctgagactctcctgtgcagcctctggattcaccttcagtgactcctacatgtcctggatccgtcaggctccagggaagggactagagtggatttcatacattggtagtagtggtaataccttttactacgcagactctgtgaagggccggttcaccatttccagagacaacgccaacaatttactgtatctgcaaatgaccagcctgagagccgaggacacggccgtgtattactgtgcgagagaagaaggcgatttttggagtgccgttgactcctggggccagggaaccctggtcaccgtctcctca

SEQ ID NO: 154 - protein, 120 aa, Artificial Sequence
  synthetic
QVQLVESGGGLVKPGGSLRLSCAASGFTFSDSYMSWIRQAPGKGLEWISYIGSSGNTFYYADSVKGRFTISRDNANNLLYLQMTSLRAEDTAVYYCAREEGDFWSAVDSWGQGTLVTVSS

SEQ ID NO: 155 - DNA, 24 bp, Artificial Sequence
  synthetic
ggattcaccttcagtgactcctac

SEQ ID NO: 156 - protein, 8 aa, Artificial Sequence
  synthetic
GFTFSDSY

SEQ ID NO: 157 - DNA, 24 bp, Artificial Sequence
  synthetic
attggtagtagtggtaataccttt

SEQ ID NO: 158 - protein, 8 aa, Artificial Sequence
  synthetic
IGSSGNTF

SEQ ID NO: 159 - DNA, 39 bp, Artificial Sequence
  synthetic
gcgagagaagaaggcgatttttggagtgccgttgactcc

SEQ ID NO: 160 - protein, 13 aa, Artificial Sequence
  synthetic
AREEGDFWSAVDS

SEQ ID NO: 161 - DNA, 321 bp, Artificial Sequence
  synthetic
gacatccagttgacccagtctccatccttcctgtctgcatctgtaggagacagagtcaccatcacttgctgggccagtcagggcattagcagttatttagcctggtatcagcaaaaaccaggtaaagcccctaaactcctgatccatactgcatccactttgcaaagtggggtcccatcaaggttcagcggcagtggatctgggacagaattcactctcacaatcagcaacctgcagcctgaagattttgcaacttattactgtcaacagcttaatagttacccattcactttcggccctgggaccaaagtggatatcaaa

SEQ ID NO: 162 - protein, 107 aa, Artificial Sequence
  synthetic
DIQLTQSPSFLSASVGDRVTITCWASQGISSYLAWYQQKPGKAPKLLIHTASTLQSGVPSRFSGSGSGTEFTLTISNLQPEDFATYYCQQLNSYPFTFGPGTKVDIK

SEQ ID NO: 163 - DNA, 18 bp, Artificial Sequence
  synthetic
cagggcattagcagttat

SEQ ID NO: 164 - protein, 6 aa, Artificial Sequence
  synthetic
QGISSY

SEQ ID NO: 165 - DNA, 9 bp, Artificial Sequence
  synthetic
actgcatcc

SEQ ID NO: 166 - protein, 3 aa, Artificial Sequence
  synthetic
TAS

SEQ ID NO: 167 - DNA, 27 bp, Artificial Sequence
  synthetic
caacagcttaatagttacccattcact

SEQ ID NO: 168 - protein, 9 aa, Artificial Sequence
  synthetic
QQLNSYPFT

SEQ ID NO: 169 - DNA, 366 bp, Artificial Sequence
  synthetic
caggtgcagctggtggagtctgggggaggcgtggtccagcctggggggtccctgagactctcctgtgcagcctctggattcaccttcggtggccatgccatgcactgggtccgccaggctccaggcaaggggctggagtggctggcagttatatcatctgatggcagtaataaacagtatgcagattctgtgaagggccgattcaccatctccagggacaatcccaagaacacgctgtatctgcaaatgaacagtctgagagttggggacacggctatttattactgtgcgaaagaggtggcacctcgttattattattacggtctggacgtctggggccaagggaccacggtcaccgtctcctca

SEQ ID NO: 170 - protein, 122 aa, Artificial Sequence
  synthetic
QVQLVESGGGVVQPGGSLRLSCAASGFTFGGHAMHWVRQAPGKGLEWLAVISSDGSNKQYADSVKGRFTISRDNPKNTLYLQMNSLRVGDTAIYYCAKEVAPRYYYYGLDVWGQGTTVTVSS

SEQ ID NO: 171 - DNA, 24 bp, Artificial Sequence
  synthetic
ggattcaccttcggtggccatgcc

SEQ ID NO: 172 - protein, 8 aa, Artificial Sequence
  synthetic
GFTFGGHA

SEQ ID NO: 173 - DNA, 24 bp, Artificial Sequence
  synthetic
atatcatctgatggcagtaataaa

SEQ ID NO: 174 - protein, 8 aa, Artificial Sequence
  synthetic
ISSDGSNK

SEQ ID NO: 175 - DNA, 45 bp, Artificial Sequence
  synthetic
gcgaaagaggtggcacctcgttattattattacggtctggacgtc

SEQ ID NO: 176 - protein, 15 aa, Artificial Sequence
  synthetic
AKEVAPRYYYYGLDV

SEQ ID NO: 177 - DNA, 318 bp, Artificial Sequence
  synthetic
gacatccagatgacccagtctccatcctccctgtctgcatctgtgggagacagagtcaccatcacttgccgggcgagtcaggacattagcaattttttagcctggtatcagcagaaaccagggaaggttcctaaactcctgatctatactgcatccactttacaatcaggggtcccatctcggttcagtggcagtggatctgggacagatttcactctcaccgtcagcagcctacagcctgaagatgttgcaacttattactgtcaaaagtatgccggcgccctcactttcggccctgggaccaaagtggatatcaaa

SEQ ID NO: 178 - protein, 106 aa, Artificial Sequence
  synthetic
DIQMTQSPSSLSASVGDRVTITCRASQDISNFLAWYQQKPGKVPKLLIYTASTLQSGVPSRFSGSGSGTDFTLTVSSLQPEDVATYYCQKYAGALTFGPGTKVDIK

SEQ ID NO: 179 - DNA, 18 bp, Artificial Sequence
  synthetic
caggacattagcaatttt

SEQ ID NO: 180 - protein, 6 aa, Artificial Sequence
  synthetic
QDISNF

SEQ ID NO: 181 - DNA, 9 bp, Artificial Sequence
  synthetic
actgcatcc

SEQ ID NO: 182 - protein, 3 aa, Artificial Sequence
  synthetic
TAS

SEQ ID NO: 183 - DNA, 24 bp, Artificial Sequence
  synthetic
caaaagtatgccggcgccctcact

SEQ ID NO: 184 - protein, 8 aa, Artificial Sequence
  synthetic
QKYAGALT

SEQ ID NO: 185 - DNA, 357 bp, Artificial Sequence
  synthetic
gaggtgcagctggtggagtctgggggaggcttggcacagcctggggggtccctgagactctcctgtgcagcctctggattcacgtttagaagctatgccatgagctgggtccgccaggctccagggaaggggccggagtgggtctcaggtataggtggtaatggtgttaccacatactacgcagactccgtgaagggccggttcaccatctccagagacaattccaagaacacgctgtttctgcaaatgaatagcctgagagccgaggacacggccgtatattattgtgtgcaggggggtttaggtggttattttacaggctactggggccagggaaccctggtcaccgtctcctca

SEQ ID NO: 186 - protein, 119 aa, Artificial Sequence
  synthetic
EVQLVESGGGLAQPGGSLRLSCAASGFTFRSYAMSWVRQAPGKGPEWVSGIGGNGVTTYYADSVKGRFTISRDNSKNTLFLQMNSLRAEDTAVYYCVQGGLGGYFTGYWGQGTLVTVSS

SEQ ID NO: 187 - DNA, 24 bp, Artificial Sequence
  synthetic
ggattcacgtttagaagctatgcc

SEQ ID NO: 188 - protein, 8 aa, Artificial Sequence
  synthetic
GFTFRSYA

SEQ ID NO: 189 - DNA, 24 bp, Artificial Sequence
  synthetic
ataggtggtaatggtgttaccaca

SEQ ID NO: 190 - protein, 8 aa, Artificial Sequence
  synthetic
IGGNGVTT

SEQ ID NO: 191 - DNA, 36 bp, Artificial Sequence
  synthetic
gtgcaggggggtttaggtggttattttacaggctac

SEQ ID NO: 192 - protein, 12 aa, Artificial Sequence
  synthetic
VQGGLGGYFTGY

SEQ ID NO: 193 - DNA, 321 bp, Artificial Sequence
  synthetic
gacatccagatgacccagtctccatcctccctgtctgcatctgtaggagacagagtcaccatcacttgccgggcaagtcagagtattagtacctatttaaattggtatcagcagaatccagggaaagcccctaaactcctgatctttgatgcatccagtttgcaaagtggggtcccatcaaggttcagtggcagtggatctgggacagatttcactctcaccatcagaggtctgcaacctgaagattttgcaacttactactgtcaacagagttacagtgccccgctcactttcggcggagggaccaaggtggagatcaaa

SEQ ID NO: 194 - protein, 107 aa, Artificial Sequence
  synthetic
DIQMTQSPSSLSASVGDRVTITCRASQSISTYLNWYQQNPGKAPKLLIFDASSLQSGVPSRFSGSGSGTDFTLTIRGLQPEDFATYYCQQSYSAPLTFGGGTKVEIK

SEQ ID NO: 195 - DNA, 18 bp, Artificial Sequence
  synthetic
cagagtattagtacctat

SEQ ID NO: 196 - protein, 6 aa, Artificial Sequence
  synthetic
QSISTY

SEQ ID NO: 197 - DNA, 9 bp, Artificial Sequence
  synthetic
gatgcatcc

SEQ ID NO: 198 - protein, 3 aa, Artificial Sequence
  synthetic
DAS

SEQ ID NO: 199 - DNA, 27 bp, Artificial Sequence
  synthetic
caacagagttacagtgccccgctcact

SEQ ID NO: 200 - protein, 9 aa, Artificial Sequence
  synthetic
QQSYSAPLT

SEQ ID NO: 201 - DNA, 357 bp, Artificial Sequence
  synthetic
caggtgcagctggtggagtctgggggaggcgtggtccagcctgggaggtccctgagactctcctgtgcagcgtctggattcaccttcagtggttatggcatgcactgggtccgccaggctccaggcaaggggctggagtgggtggcacttatatggcttgatggaagtaatgactactatgcagactccgtgaagggccgattcaccatctccagagacaattccaagaacacgttatatctgcaaatgaacagactgagagccgaggacacggctgtgtattactgtgcgagagatggcccggttgctgctatacccgactactggggccagggaaccctggtcaccgtctcctca

SEQ ID NO: 202 - protein, 119 aa, Artificial Sequence
  synthetic
QVQLVESGGGVVQPGRSLRLSCAASGFTFSGYGMHWVRQAPGKGLEWVALIWLDGSNDYYADSVKGRFTISRDNSKNTLYLQMNRLRAEDTAVYYCARDGPVAAIPDYWGQGTLVTVSS

SEQ ID NO: 203 - DNA, 24 bp, Artificial Sequence
  synthetic
ggattcaccttcagtggttatggc

SEQ ID NO: 204 - protein, 8 aa, Artificial Sequence
  synthetic
GFTFSGYG

SEQ ID NO: 205 - DNA, 24 bp, Artificial Sequence
  synthetic
atatggcttgatggaagtaatgac

SEQ ID NO: 206 - protein, 8 aa, Artificial Sequence
  synthetic
IWLDGSND

SEQ ID NO: 207 - DNA, 36 bp, Artificial Sequence
  synthetic
gcgagagatggcccggttgctgctatacccgactac

SEQ ID NO: 208 - protein, 12 aa, Artificial Sequence
  synthetic
ARDGPVAAIPDY

SEQ ID NO: 209 - DNA, 321 bp, Artificial Sequence
  synthetic
gacatccagatgacccagtctccttccaccctgtctgcatctgtaggagacagagtcaccatcacttgccgggccagtcagagtattagtaggtggttggcctggtatcagctgaaaccagggaaagcccctaagctcctgatctataaggcgtctagtttagaaagtggggtcccatcaaggttcagcggcagtggatctgggacagacttcactctcaccatcagcagcctgcaacctgatgattttgcaacttattactgccaacagtataatacttattcgtacacttttggccaggggaccaagctggagatcaaa

SEQ ID NO: 210 - protein, 107 aa, Artificial Sequence
  synthetic
DIQMTQSPSTLSASVGDRVTITCRASQSISRWLAWYQLKPGKAPKLLIYKASSLESGVPSRFSGSGSGTDFTLTISSLQPDDFATYYCQQYNTYSYTFGQGTKLEIK

SEQ ID NO: 211 - DNA, 18 bp, Artificial Sequence
  synthetic
cagagtattagtaggtgg

SEQ ID NO: 212 - protein, 6 aa, Artificial Sequence
  synthetic
QSISRW

SEQ ID NO: 213 - DNA, 9 bp, Artificial Sequence
  synthetic
aaggcgtct

SEQ ID NO: 214 - protein, 3 aa, Artificial Sequence
  synthetic
KAS

SEQ ID NO: 215 - DNA, 27 bp, Artificial Sequence
  synthetic
caacagtataatacttattcgtacact

SEQ ID NO: 216 - protein, 9 aa, Artificial Sequence
  synthetic
QQYNTYSYT

SEQ ID NO: 217 - DNA, 363 bp, Artificial Sequence
  synthetic
gaggtgcagctggtggagtctgggggaggtgtggtacggcctggggggtccctgagactctcctgtgcagcctctggattcacctttgatgaatatggcatgacttgggtccgccaagttccagggaaggggctggagtgggtctctggtattacttggaatggtggtttcacagattatacagactctgtgaagggccgattcaccagctccagagacaacgccaagaactccctgtatctgcaaatgaacagtctgagagccgaggacacggccttgtattactgtgcgagagatggatatagcagctcgtggggggcttatgatatatggggccaagggacaatggtcaccgtctcttca

SEQ ID NO: 218 - protein, 121 aa, Artificial Sequence
  synthetic
EVQLVESGGGVVRPGGSLRLSCAASGFTFDEYGMTWVRQVPGKGLEWVSGITWNGGFTDYTDSVKGRFTSSRDNAKNSLYLQMNSLRAEDTALYYCARDGYSSSWGAYDIWGQGTMVTVSS

SEQ ID NO: 219 - DNA, 24 bp, Artificial Sequence
  synthetic
ggattcacctttgatgaatatggc

SEQ ID NO: 220 - protein, 8 aa, Artificial Sequence
  synthetic
GFTFDEYG

SEQ ID NO: 221 - DNA, 24 bp, Artificial Sequence
  synthetic
attacttggaatggtggtttcaca

SEQ ID NO: 222 - protein, 8 aa, Artificial Sequence
  synthetic
ITWNGGFT

SEQ ID NO: 223 - DNA, 42 bp, Artificial Sequence
  synthetic
gcgagagatggatatagcagctcgtggggggcttatgatata

SEQ ID NO: 224 - protein, 14 aa, Artificial Sequence
  synthetic
ARDGYSSSWGAYDI

SEQ ID NO: 225 - DNA, 321 bp, Artificial Sequence
  synthetic
gacatccagatgacccagtctccatcatccctgtctgcatctgtgggagacagagtcaccatcacttgccgggcaagtcagagcattagcacctatttaaattggtatcagcagaaaccagggaaagcccctaagctcctgatctatgctgcatccagtttgcaaagtggggtcccattaaggttcagtggcagtggatctgggactgatttcactctcaccatcagcagtctgcaacctgaagattttgcaagttatttctgtcaacagagttacagtaccccgtacacttttggccaggggaccaagctggagatcaaa

SEQ ID NO: 226 - protein, 107 aa, Artificial Sequence
  synthetic
DIQMTQSPSSLSASVGDRVTITCRASQSISTYLNWYQQKPGKAPKLLIYAASSLQSGVPLRFSGSGSGTDFTLTISSLQPEDFASYFCQQSYSTPYTFGQGTKLEIK

SEQ ID NO: 227 - DNA, 18 bp, Artificial Sequence
  synthetic
cagagcattagcacctat

SEQ ID NO: 228 - protein, 6 aa, Artificial Sequence
  synthetic
QSISTY

SEQ ID NO: 229 - DNA, 9 bp, Artificial Sequence
  synthetic
gctgcatcc

SEQ ID NO: 230 - protein, 3 aa, Artificial Sequence
  synthetic
AAS

SEQ ID NO: 231 - DNA, 27 bp, Artificial Sequence
  synthetic
caacagagttacagtaccccgtacact

SEQ ID NO: 232 - protein, 9 aa, Artificial Sequence
  synthetic
QQSYSTPYT

SEQ ID NO: 233 - DNA, 360 bp, Artificial Sequence
  synthetic
gaagtgcagctggtggagtctgggggaggcgtggtacagcctggggggtccctgagactctcctgtgcagcctctggattcacctttaatgattatgccatgcactgggtccgtcaagctccagggaagggtctggagtgggtctctcttattagtggagatggtggtaacacatactatgcagactctgtgaagggccgactcaccatctccagagacaacagcaaaaactccctgtatctgcaaatgaacagtctgagaacagaggacaccgccttatattactgtgcaaaagataagggctggaacttcggttacttcgatctctggggccgtggcaccctggtcactgtctcctca

SEQ ID NO: 234 - protein, 120 aa, Artificial Sequence
  synthetic
EVQLVESGGGVVQPGGSLRLSCAASGFTFNDYAMHWVRQAPGKGLEWVSLISGDGGNTYYADSVKGRLTISRDNSKNSLYLQMNSLRTEDTALYYCAKDKGWNFGYFDLWGRGTLVTVSS

SEQ ID NO: 235 - DNA, 24 bp, Artificial Sequence
  synthetic
ggattcacctttaatgattatgcc

SEQ ID NO: 236 - protein, 8 aa, Artificial Sequence
  synthetic
GFTFNDYA

SEQ ID NO: 237 - DNA, 24 bp, Artificial Sequence
  synthetic
attagtggagatggtggtaacaca

SEQ ID NO: 238 - protein, 8 aa, Artificial Sequence
  synthetic
ISGDGGNT

SEQ ID NO: 239 - DNA, 39 bp, Artificial Sequence
  synthetic
gcaaaagataagggctggaacttcggttacttcgatctc

SEQ ID NO: 240 - protein, 13 aa, Artificial Sequence
  synthetic
AKDKGWNFGYFDL

SEQ ID NO: 241 - DNA, 327 bp, Artificial Sequence
  synthetic
gacatccagatgacccagtctccatcctccctgtctacatctgtgggagacagagtcaccatcacttgccgggcaagtcagaacattgacacctatttaaattggtatcagcagaaaccagggaaagcccctaaactcctgatctatgatgcatccagtttacaaagtggggtcccatcacggttcagtggcagcggatctgggacagatttcactctcaccatcaccagtctgcaacctgaagattttgccacttactactgtcaacagaatgacaatattcttcaccctctcactttcggcggagggaccaaggtggagatcaaa

SEQ ID NO: 242 - protein, 109 aa, Artificial Sequence
  synthetic
DIQMTQSPSSLSTSVGDRVTITCRASQNIDTYLNWYQQKPGKAPKLLIYDASSLQSGVPSRFSGSGSGTDFTLTITSLQPEDFATYYCQQNDNILHPLTFGGGTKVEIK

SEQ ID NO: 243 - DNA, 18 bp, Artificial Sequence
  synthetic
cagaacattgacacctat

SEQ ID NO: 244 - protein, 6 aa, Artificial Sequence
  synthetic
QNIDTY

SEQ ID NO: 245 - DNA, 9 bp, Artificial Sequence
  synthetic
gatgcatcc

SEQ ID NO: 246 - protein, 3 aa, Artificial Sequence
  synthetic
DAS

SEQ ID NO: 247 - DNA, 33 bp, Artificial Sequence
  synthetic
caacagaatgacaatattcttcaccctctcact

SEQ ID NO: 248 - protein, 11 aa, Artificial Sequence
  synthetic
QQNDNILHPLT

SEQ ID NO: 249 - DNA, 369 bp, Artificial Sequence
  synthetic
gaggtgcagctggtggagtctgggggaggcttggtccaaccgggggggtccctgagactctcctgtgcagcctctggattccactctaatagatattggatggactgggtccgccaggctccagggaaggggctggagtgggtggccaacataaagcaagatggaagtgaggaaaactatgtggactctgtgaagggccgattcaccatctccagagacaacgccaagaactcactttatctgcaaatgaacagcctgagagccgaggacacggctgtgtattactgtgcgagagatcgaagcacctcgtgggtcccttactggttcttcgatctctggggccgtggcaccctggtcactgtctcctca

SEQ ID NO: 250 - protein, 123 aa, Artificial Sequence
  synthetic
EVQLVESGGGLVQPGGSLRLSCAASGFHSNRYWMDWVRQAPGKGLEWVANIKQDGSEENYVDSVKGRFTISRDNAKNSLYLQMNSLRAEDTAVYYCARDRSTSWVPYWFFDLWGRGTLVTVSS

SEQ ID NO: 251 - DNA, 24 bp, Artificial Sequence
  synthetic
ggattccactctaatagatattgg

SEQ ID NO: 252 - protein, 8 aa, Artificial Sequence
  synthetic
GFHSNRYW

SEQ ID NO: 253 - DNA, 24 bp, Artificial Sequence
  synthetic
ataaagcaagatggaagtgaggaa

SEQ ID NO: 254 - protein, 8 aa, Artificial Sequence
  synthetic
IKQDGSEE

SEQ ID NO: 255 - DNA, 48 bp, Artificial Sequence
  synthetic
gcgagagatcgaagcacctcgtgggtcccttactggttcttcgatctc

SEQ ID NO: 256 - protein, 16 aa, Artificial Sequence
  synthetic
ARDRSTSWVPYWFFDL

SEQ ID NO: 257 - DNA, 324 bp, Artificial Sequence
  synthetic
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 258 - protein, 108 aa, Artificial Sequence
  synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 259 - DNA, 18 bp, Artificial Sequence
  synthetic
cagagcattagcagctat

SEQ ID NO: 260 - protein, 6 aa, Artificial Sequence
  synthetic
QSISSY

SEQ ID NO: 261 - DNA, 9 bp, Artificial Sequence
  synthetic
gctgcatcc

SEQ ID NO: 262 - protein, 3 aa, Artificial Sequence
  synthetic
AAS

SEQ ID NO: 263 - DNA, 30 bp, Artificial Sequence
  synthetic
caacagagttacagtacccctccgatcacc

SEQ ID NO: 264 - protein, 10 aa, Artificial Sequence
  synthetic
QQSYSTPPIT

SEQ ID NO: 265 - DNA, 360 bp, Artificial Sequence
  synthetic
gaagtgcagctggtggagtctgggggaggcgtggtacagcggggggagtccctgagactctcctgttcagcctctgacttcatctttaaagattatgccatgtactgggtccgtcaaattccagggaagggtctagagtggatctctcttattagtggtgatggtgacactacatggtatggagactctgtgaagggccgattcaccatctccagagacaacaacgaaaactccctctttctgcaaatgaacgatctgagaactgaggacaccgccatgtactactgtgcaagagatatggggtggaacttctttcagttgcaatactggggccagggaaccctggtcaccgtctcctca

SEQ ID NO: 266 - protein, 120 aa, Artificial Sequence
  synthetic
EVQLVESGGGVVQRGESLRLSCSASDFIFKDYAMYWVRQIPGKGLEWISLISGDGDTTWYGDSVKGRFTISRDNNENSLFLQMNDLRTEDTAMYYCARDMGWNFFQLQYWGQGTLVTVSS

SEQ ID NO: 267 - DNA, 24 bp, Artificial Sequence
  synthetic
gacttcatctttaaagattatgcc

SEQ ID NO: 268 - protein, 8 aa, Artificial Sequence
  synthetic
DFIFKDYA

SEQ ID NO: 269 - DNA, 24 bp, Artificial Sequence
  synthetic
attagtggtgatggtgacactaca

SEQ ID NO: 270 - protein, 8 aa, Artificial Sequence
  synthetic
ISGDGDTT

SEQ ID NO: 271 - DNA, 39 bp, Artificial Sequence
  synthetic
gcaagagatatggggtggaacttctttcagttgcaatac

SEQ ID NO: 272 - protein, 13 aa, Artificial Sequence
  synthetic
ARDMGWNFFQLQY

SEQ ID NO: 273 - DNA, 361 bp, Artificial Sequence
  synthetic
dcaggtgcagctgcaggagtcgggccccgcactggtgaagccttcacagaccctgtccctcacctgcactgtctctggtggctccatcatcagaggtagtacctactggagttgggtccgccaattcccagggaagggcctggagtggattggatacagttattacagtgggaccgcctactataatccgtccctcgagagtcgagctaccatttctgtagacacgtctaagaaccagttctccctgaacctgaagtctgtgacggccgcggacacggccgtgtattattgtacaagagaaataggagtggctggtctctttgacatctggggccagggaaccctggtcaccgtctcctca

SEQ ID NO: 274 - protein, 120 aa, Artificial Sequence
  synthetic
QVQLQESGPALVKPSQTLSLTCTVSGGSIIRGSTYWSWVRQFPGKGLEWIGYSYYSGTAYYNPSLESRATISVDTSKNQFSLNLKSVTAADTAVYYCTREIGVAGLFDIWGQGTLVTVSS

SEQ ID NO: 275 - DNA, 30 bp, Artificial Sequence
  synthetic
ggtggctccatcatcagaggtagtacctac

SEQ ID NO: 276 - protein, 10 aa, Artificial Sequence
  synthetic
GGSIIRGSTY

SEQ ID NO: 277 - DNA, 21 bp, Artificial Sequence
  synthetic
agttattacagtgggaccgcc

SEQ ID NO: 278 - protein, 7 aa, Artificial Sequence
  synthetic
SYYSGTA

SEQ ID NO: 279 - DNA, 36 bp, Artificial Sequence
  synthetic
acaagagaaataggagtggctggtctctttgacatc

SEQ ID NO: 280 - protein, 12 aa, Artificial Sequence
  synthetic
TREIGVAGLFDI

SEQ ID NO: 281 - DNA, 324 bp, Artificial Sequence
  synthetic
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 282 - protein, 108 aa, Artificial Sequence
  synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 283 - DNA, 36 bp, Artificial Sequence
  synthetic
agagcaagtcagtcagtctctagctcttatctcgcc

SEQ ID NO: 284 - protein, 12 aa, Artificial Sequence
  synthetic
RASQSVSSSYLA

SEQ ID NO: 285 - DNA, 21 bp, Artificial Sequence
  synthetic
ggggcaagttccagggccacc

SEQ ID NO: 286 - protein, 7 aa, Artificial Sequence
  synthetic
GASSRAT

SEQ ID NO: 287 - DNA, 27 bp, Artificial Sequence
  synthetic
caacagtacggaagcagcccgtggacg

SEQ ID NO: 288 - protein, 9 aa, Artificial Sequence
  synthetic
QQYGSSPWT

SEQ ID NO: 289 - DNA, 357 bp, Artificial Sequence
  synthetic
caggagcagttggtgcagtctggggctgaggtgaagaagcctggggcctcagtgaaggtctcctgtaaggcttctggatacaccttcaccggctactatatacattgggtgcgacaggcccctggactagggcttgaatggatgggatggatcaaccctaacagtggtggcacaaaatatgcacagaagtttcagggcagggtcaccatgaccagggacacgtccatcaatacagcctacatggagctgaaaagactgaaatctgacgactcggccgtatattactgtgcgagagacgcccctccccatgatgtttttgatatctggggccaagggacattggtcaccgtctcttca

SEQ ID NO: 290 - protein, 119 aa, Artificial Sequence
  synthetic
QEQLVQSGAEVKKPGASVKVSCKASGYTFTGYYIHWVRQAPGLGLEWMGWINPNSGGTKYAQKFQGRVTMTRDTSINTAYMELKRLKSDDSAVYYCARDAPPHDVFDIWGQGTLVTVSS

SEQ ID NO: 291 - DNA, 24 bp, Artificial Sequence
  synthetic
ggatacaccttcaccggctactat

SEQ ID NO: 292 - protein, 8 aa, Artificial Sequence
  synthetic
GYTFTGYY

SEQ ID NO: 293 - DNA, 24 bp, Artificial Sequence
  synthetic
atcaaccctaacagtggtggcaca

SEQ ID NO: 294 - protein, 8 aa, Artificial Sequence
  synthetic
INPNSGGT

SEQ ID NO: 295 - DNA, 36 bp, Artificial Sequence
  synthetic
gcgagagacgcccctccccatgatgtttttgatatc

SEQ ID NO: 296 - protein, 12 aa, Artificial Sequence
  synthetic
ARDAPPHDVFDI

SEQ ID NO: 297 - DNA, 321 bp, Artificial Sequence
  synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaattggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaa

SEQ ID NO: 298 - protein, 107 aa, Artificial Sequence
  synthetic
DIQMTQSPSSLSASVGDRVTITCRASQGIRNDLGWYQQKPGKAPKRLIYAASSLQIGVPSRFSGSGSGTEFTLTISSLQPEDFATYYCLQHNSYPLTFGGGTKVEIK

SEQ ID NO: 299 - DNA, 18 bp, Artificial Sequence
  synthetic
cagggcattagaaatgat

SEQ ID NO: 300 - protein, 6 aa, Artificial Sequence
  synthetic
QGIRND

SEQ ID NO: 301 - DNA, 9 bp, Artificial Sequence
  synthetic
gctgcatcc

SEQ ID NO: 302 - protein, 3 aa, Artificial Sequence
  synthetic
AAS

SEQ ID NO: 303 - DNA, 27 bp, Artificial Sequence
  synthetic
ctacagcataatagttacccgctcact

SEQ ID NO: 304 - protein, 9 aa, Artificial Sequence
  synthetic
LQHNSYPLT

SEQ ID NO: 305 - DNA, 357 bp, Artificial Sequence
  synthetic
caggtgcagctgcaagagtcgggcccaggactggtgaagccttcacagaccctgtccctcacctgcactgtctctggtggctccatcagtagtggtgcttaccactggagctggatccgccagcacccagggaagggcctagagtggattggatacatctattacaatggggacacctactataatccgtccctcaagagtcgcgttaccatttcagtggacacgtctaagaaccaattcttcctgaaggtgacctctgtgactgccgcggacacggccatgtattactgtgcgggagaaaagcagctgactgcttttgatatctggggccaagggacattggtcaccgtctcttca

SEQ ID NO: 306 - protein, 119 aa, Artificial Sequence
  synthetic
QVQLQESGPGLVKPSQTLSLTCTVSGGSISSGAYHWSWIRQHPGKGLEWIGYIYYNGDTYYNPSLKSRVTISVDTSKNQFFLKVTSVTAADTAMYYCAGEKQLTAFDIWGQGTLVTVSS

SEQ ID NO: 307 - DNA, 30 bp, Artificial Sequence
  synthetic
ggtggctccatcagtagtggtgcttaccac

SEQ ID NO: 308 - protein, 10 aa, Artificial Sequence
  synthetic
GGSISSGAYH

SEQ ID NO: 309 - DNA, 21 bp, Artificial Sequence
  synthetic
atctattacaatggggacacc

SEQ ID NO: 310 - protein, 7 aa, Artificial Sequence
  synthetic
IYYNGDT

SEQ ID NO: 311 - DNA, 33 bp, Artificial Sequence
  synthetic
gcgggagaaaagcagctgactgcttttgatatc

SEQ ID NO: 312 - protein, 11 aa, Artificial Sequence
  synthetic
AGEKQLTAFDI

SEQ ID NO: 313 - DNA, 321 bp, Artificial Sequence
  synthetic
gtcatccagatgacccagtctccatcctccctgtctgcatctgttggagacagagtcaccataacttgccgggcgagtcaggacattaataattttttaaattggtatcaacagaaattagggaaagcccctaaactcctgatctccgatgcatccaatttgcagacaggagtcccgtcaaggttcagtggaagtggatctgggacagattttactttcaccatcagcagcctgcagcctgaagatattgctgcatattactgtcaacaatatgatcatttcccgtatacttttggccaggggaccagactggagaacaat

SEQ ID NO: 314 - protein, 107 aa, Artificial Sequence
  synthetic
VIQMTQSPSSLSASVGDRVTITCRASQDINNFLNWYQQKLGKAPKLLISDASNLQTGVPSRFSGSGSGTDFTFTISSLQPEDIAAYYCQQYDHFPYTFGQGTRLENN

SEQ ID NO: 315 - DNA, 18 bp, Artificial Sequence
  synthetic
caggacattaataatttt

SEQ ID NO: 316 - protein, 6 aa, Artificial Sequence
  synthetic
QDINNF

SEQ ID NO: 317 - DNA, 9 bp, Artificial Sequence
  synthetic
gatgcatcc

SEQ ID NO: 318 - protein, 3 aa, Artificial Sequence
  synthetic
DAS

SEQ ID NO: 319 - DNA, 27 bp, Artificial Sequence
  synthetic
caacaatatgatcatttcccgtatact

SEQ ID NO: 320 - protein, 9 aa, Artificial Sequence
  synthetic
QQYDHFPYT

SEQ ID NO: 321 - DNA, 357 bp, Artificial Sequence
  synthetic
gaggtgcagttggtggagtctgggggaggtgtggttcggcctggggggtccctgagactctcctgtgcagcctctggattcacctttgatgattatggcatgacctgggtccgccaagctccagggaaggggctggagtgggtctctggtattaattggaatggcgatagcacagagtattcagactctgtgaagggccgattcaccatctccagagacaacgccaagaactccctgtatctgcaaatgaacagtctgagagccgaggacacggccttctatcactgtgcgagagagaataactggaacttctactttgactactggggccagggaaccctggtcaccgtctcctca

SEQ ID NO: 322 - protein, 119 aa, Artificial Sequence
  synthetic
EVQLVESGGGVVRPGGSLRLSCAASGFTFDDYGMTWVRQAPGKGLEWVSGINWNGDSTEYSDSVKGRFTISRDNAKNSLYLQMNSLRAEDTAFYHCARENNWNFYFDYWGQGTLVTVSS

SEQ ID NO: 323 - DNA, 24 bp, Artificial Sequence
  synthetic
ggattcacctttgatgattatggc

SEQ ID NO: 324 - protein, 8 aa, Artificial Sequence
  synthetic
GFTFDDYG

SEQ ID NO: 325 - DNA, 24 bp, Artificial Sequence
  synthetic
attaattggaatggcgatagcaca

SEQ ID NO: 326 - protein, 8 aa, Artificial Sequence
  synthetic
INWNGDST

SEQ ID NO: 327 - DNA, 36 bp, Artificial Sequence
  synthetic
gcgagagagaataactggaacttctactttgactac

SEQ ID NO: 328 - protein, 12 aa, Artificial Sequence
  synthetic
ARENNWNFYFDY

SEQ ID NO: 329 - DNA, 321 bp, Artificial Sequence
  synthetic
gaaatagtgatgacgcagtctccagccaccctgtctgtgtctcgaggggaaagagccaccctctcctgtagggccagtcagagtgttagcagcaacttagcctggtaccagcagaaacttggccaggctcccaggctcctcatctatggtgcatccaccagggccactggtatcccagccaggttcagtggcagtgggtctgggacagagttcactctcaccatcagcagcctgcagtctgaagattttgcagtttattattgtcagcagtataataactggccgtggacgttcggccaagggaccaaggtggaaatcaaa

SEQ ID NO: 330 - protein, 107 aa, Artificial Sequence
  synthetic
EIVMTQSPATLSVSRGERATLSCRASQSVSSNLAWYQQKLGQAPRLLIYGASTRATGIPARFSGSGSGTEFTLTISSLQSEDFAVYYCQQYNNWPWTFGQGTKVEIK

SEQ ID NO: 331 - DNA, 18 bp, Artificial Sequence
  synthetic
cagagtgttagcagcaac

SEQ ID NO: 332 - protein, 6 aa, Artificial Sequence
  synthetic
QSVSSN

SEQ ID NO: 333 - DNA, 9 bp, Artificial Sequence
  synthetic
ggtgcatcc

SEQ ID NO: 334 - protein, 3 aa, Artificial Sequence
  synthetic
GAS

SEQ ID NO: 335 - DNA, 27 bp, Artificial Sequence
  synthetic
cagcagtataataactggccgtggacg

SEQ ID NO: 336 - protein, 9 aa, Artificial Sequence
  synthetic
QQYNNWPWT

SEQ ID NO: 337 - DNA, 351 bp, Artificial Sequence
  synthetic
caggtccacctggtacagtctggggctgaggtgaagaagcctggggcctcagtgaaggtctcctgcaaggtttccggaaacaccctcactgaattatccatgcactgggtgcgacaggctcctggaaaagggcttgagtggatgggaggttttgatcctgaagatggtgacacaatctactcacagaagttccagggcagagtcaccttgaccgaggacacatctacagacacagcctacatggagctgagcagcctgagatctgaggacacggccgtgtattactgttcaacagtggggggacctacctctgactgctggggccagggaaccctggtcaccgtctcctca

SEQ ID NO: 338 - protein, 117 aa, Artificial Sequence
  synthetic
QVHLVQSGAEVKKPGASVKVSCKVSGNTLTELSMHWVRQAPGKGLEWMGGFDPEDGDTIYSQKFQGRVTLTEDTSTDTAYMELSSLRSEDTAVYYCSTVGGPTSDCWGQGTLVTVSS

SEQ ID NO: 339 - DNA, 24 bp, Artificial Sequence
  synthetic
ggaaacaccctcactgaattatcc

SEQ ID NO: 340 - protein, 8 aa, Artificial Sequence
  synthetic
GNTLTELS

SEQ ID NO: 341 - DNA, 24 bp, Artificial Sequence
  synthetic
tttgatcctgaagatggtgacaca

SEQ ID NO: 342 - protein, 8 aa, Artificial Sequence
  synthetic
FDPEDGDT

SEQ ID NO: 343 - DNA, 30 bp, Artificial Sequence
  synthetic
tcaacagtggggggacctacctctgactgc

SEQ ID NO: 344 - protein, 10 aa, Artificial Sequence
  synthetic
STVGGPTSDC

SEQ ID NO: 345 - DNA, 321 bp, Artificial Sequence
  synthetic
gacatccagatgacccagtctccatcctccctgtctgcatctgtaggagacagagtcaccatcacttgccaggcgagtcaggacattagcaactatttaaattggtatcagcagaaaccagggaaagcccctaaggtcctgatcttcgatgcatccaatttagaaccaggggtcccatcaaggttcagtggaagtggatctgggacagattttactttcaccatcatcagcctgcagcctgaagatattgcaacatattactgtcaacaatatgataatctcccgatcaccttcggccaggggacacgactggacattaaa

SEQ ID NO: 346 - protein, 107 aa, Artificial Sequence
  synthetic
DIQMTQSPSSLSASVGDRVTITCQASQDISNYLNWYQQKPGKAPKVLIFDASNLEPGVPSRFSGSGSGTDFTFTIISLQPEDIATYYCQQYDNLPITFGQGTRLDIK

SEQ ID NO: 347 - DNA, 18 bp, Artificial Sequence
  synthetic
caggacattagcaactat

SEQ ID NO: 348 - protein, 6 aa, Artificial Sequence
  synthetic
QDISNY

SEQ ID NO: 349 - DNA, 9 bp, Artificial Sequence
  synthetic
gatgcatcc

SEQ ID NO: 350 - protein, 3 aa, Artificial Sequence
  synthetic
DAS

SEQ ID NO: 351 - DNA, 27 bp, Artificial Sequence
  synthetic
caacaatatgataatctcccgatcacc

SEQ ID NO: 352 - protein, 9 aa, Artificial Sequence
  synthetic
QQYDNLPIT

SEQ ID NO: 353 - protein, 9 aa, Homo sapiens
NMATGMDSW

SEQ ID NO: 354 - protein, 20 aa, Homo sapiens
WEVHLVPRRKQLQFALPDSL

SEQ ID NO: 355 - protein, 18 aa, Homo sapiens
KDMQLGRLHMKTLLPVSK

SEQ ID NO: 356 - protein, 9 aa, Homo sapiens
NDETCEQRA

SEQ ID NO: 357 - protein, 7 aa, Homo sapiens
SHKDMQL

SEQ ID NO: 358 - protein, 448 aa, Artificial Sequence
  Humanized mouse immunoglobulin
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 214 aa, Artificial Sequence [sequence omitted]

SEQ ID NO: 360 - protein, 448 aa, Artificial Sequence
  Humanized mouse immunoglobulin
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 361 - protein, 214 aa, Artificial Sequence [sequence omitted]

SEQ ID NO: 362 - protein, 1676 aa, Homo sapiens
  mat_peptide (19)..(1676)
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 363 - protein, 1658 aa, Homo sapiens
QEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVHQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 364 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 365 - protein, 96 aa, Homo sapiens
MTVARPSVPAALPLLGELPRLLLLVLLCLPAVWGDCGLPPDVPNAQPALALAVQVFPRILRTNVKKALKFLARRTQSALRAVNGQILKSSAIVAAR

SEQ ID NO: 366 - protein, 96 aa, Homo sapiens
MTVARPSVPAALPLLGELPRLLLLVLLCLPAVWGDCAFPQMYLMPSQLWKAVQVFPRILRTNVKKALKFLARRTQSALRAVNGQILKSSAIVAARC

SEQ ID NO: 367 - protein, 100 aa, Homo sapiens
SSFCLISGSSVQWSDPLPECREIYCPAPPQIDNGIIQGERDHYGYRQSVTYACNKGFTMIGEHSIYSTVNNDEGEWSGPPPECRGKSLTSKVPPTVQKPT

SEQ ID NO: 368 - protein, 447 aa, Homo sapiens
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 369 - protein, 214 aa, Homo sapiens
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 370 - RNA, 23 nt, Artificial Sequence
  therapeutic oligonucleotide
uauuauaaaaauaucuugcuuuu

SEQ ID NO: 371 - RNA, 21 nt, Artificial Sequence
  therapeutic oligonucleotide
aagcaagauauuuuuauaaua

SEQ ID NO: 372 - DNA, 21 bp, Artificial Sequence
  therapeutic oligonucleotide
aagcaagauauuuuuauaaua

SEQ ID NO: 373 - DNA, 25 bp, Artificial Sequence
  therapeutic oligonucleotide
uauuauaaaaauaucuugcuuuutt

(19) United States
(12) Patent Application Publication
(10) Pub. No.: US 2021/0139573 A1
(43) Pub. Date: May 13, 2021

(54) DOSING REGIMENS FOR TREATING OR PREVENTING C5-ASSOCIATED DISEASES

(71) Applicant: Regeneron Pharmaceuticals, Inc., Tarrytown, NY (US)

(72) Inventors: John Davis, Scarsdale, NY (US); Albert Thomas Dicioccio, Madison, CT (US); Olivier Harari, New York, NY (US); Kuan-Ju Lin, Stamford, CT (US); Andrew Rankin, Stamford, CT (US); Ronda Rippley, Charlestown, MA (US); Jonathan Weyne, Chappaqua, NY (US); George Yancopoulos, Yorktown Heights, NY (US); Feng Yang, Chappaqua, NY (US); Yi Zhang, Scarsdale, NY (US)

(21) Appl. No.: 17/078,309

(22) Filed: Oct. 23, 2020

Related U.S. Application Data
(60) Provisional application No. 63/019,533, filed on May 4, 2020.
(60) Provisional application No. 62/992,330, filed on Mar. 20, 2020.
(60) Provisional application No. 62/926,213, filed on Oct. 25, 2019.

(51) Int. Cl. C07K 16/18; A61P 7/00; A61K 45/06

(52) U.S. Cl. CPC C07K 16/18; A61K 2039/505; A61K 45/06; A61P 7/00

(57) ABSTRACT

The present invention provides dosing regimens of anti-C5 antibodies, such as pozelimab, for treating or preventing C5-associated diseases such as paroxysmal nocturnal hemoglobinuria or CHAPLE disease.

This application claims the benefit of U.S. Provisional Patent Application No. 62/926,213, filed Oct. 25, 2019; U.S. Provisional Patent Application No. 62/992,330, filed Mar. 20, 2020; and U.S. Provisional Patent Application No. 63/019,533, filed May 4, 2020; each of which is herein incorporated by reference in its entirety for all purposes.

The sequence listing of the present application is submitted electronically as an ASCII formatted sequence listing with a file name “seqlist10673P2”, creation date of Mar. 20, 2020, and a size of 165 Kb. This sequence listing submitted is part of the specification and is herein incorporated by reference in its entirety.

### FIELD OF THE INVENTION

The present invention relates to methods for administering an antagonist anti-C5 antibody to treat or prevent a C5-related disorder.

### BACKGROUND OF THE INVENTION

Paroxysmal nocturnal hemoglobinuria (PNH) is a chronic, progressive, life-threatening, and rare multisystem disease. Typically, it is characterized by uncontrolled complement activation on red blood cells (RBCs), resulting in intravascular hemolysis (Sahin et al., Pesg PNH diagnosis, follow-up and treatment guidelines. Am J Blood Res 2016; 6(2):19-27), and on white blood cells (WBCs) and platelets, resulting in an increased risk of thrombosis. The estimated incidence of PNH is 1.3 cases per million individuals per year, and the estimated prevalence is 15.9 cases per million individuals per year (Preis & Lowrey, Laboratory tests for paroxysmal nocturnal hemoglobinuria. Am J Hematol 2014; 89(3):339-41).

Paroxysmal nocturnal hemoglobinuria (PNH) is a rare acquired, life-threatening disease of the blood. Defective red blood cells of PNH are extremely susceptible to premature destruction by a particular part of a person's own immune system called the complement system. The disease is characterized by destruction of red blood cells (hemolytic anemia), blood clots (thrombosis), and impaired bone marrow function.

CD55-deficient protein-losing enteropathy (CD55-deficient PLE) is a rare disease, also referred to as complement hyperactivation, angiopathic thrombosis, protein-losing enteropathy (CHAPLE disease), that can be treated by C5 blockade (Kurolap et al., Loss of CD55 in Eculizumab-Responsive Protein-Losing Enteropathy. N Engl J Med., 377(1):87-89 (2017); Ozen et al., CD55 Deficiency and Protein-Losing Enteropathy. N Engl J Med., 377(15):1499-500 (2017)). CD55-deficient PLE/CHAPLE disease is caused by biallelic loss-of-function mutations in the CD55 gene. The absence of CD55 causes overactivation of the complement system, causing the production of various complement products including anaphylatoxins and the membrane-attack complex. In CD55-deficient PLE, isolated germ line loss of CD55 expression in all tissues manifests in the GI tract, as primary intestinal lymphangiectasia, which causes PLE. The majority of patients suffer from early-onset GI manifestations, including bloody diarrhea, vomiting, and abdominal pain, and occasionally develop partial or complete intestinal obstruction and intestinal failure.

Eculizumab is an antibody directed against C5 which blocks the formation of the MAC-C5b-9, thus protecting PNH RBCs from complement-mediated intravascular hemolysis. However, not all patients receive optimal therapeutic benefit. For example, 25% of patients still need recurrent, albeit less frequent, blood transfusions. Up to 20% of patients on eculizumab therapy require significant increases in dose or dose frequency due to breakthrough hemolysis secondary to incomplete inhibition of C5 (Nakayama et al., Eculizumab Dosing Intervals Longer than 17 Days May Be Associated with Greater Risk of Breakthrough Hemolysis in Patients with Paroxysmal Nocturnal Hemoglobinuria. Biol Pharm Bull 2016; 39(2):285-8) (Hill et al., Thrombosis in paroxysmal nocturnal hemoglobinuria. Blood 2013; 121(25):4985-96) (Peffault de Latour et al., Assessing complement blockade in patients with paroxysmal nocturnal hemoglobinuria receiving eculizumab. Blood 2015; 125(5):775-83). Also, eculizumab administration every 2 weeks (Q2W) by intravenous (IV) infusion is described as burdensome for patients. Ravulizumab is also an anti-C5 antibody for treating diseases such as PNH. Some patients using ravulizumab, however, still experience some hemolytic breakthrough. In addition, IV administered ravulizumab does not offer the significant convenience and reduced burden of subcutaneous (SC) self-administration (as originally approved by the US FDA). Subcutaneous ravulizumab dosage regimens call for injection of 7 ml in two separate injections over a 10 minute time period. Alexion: Investor Day (slide deck), Mar. 20, 2019.

### SUMMARY OF THE INVENTION

The present invention provides methods for administering an antagonist antigen-binding protein that binds specifically to C5 (e.g., REGN3918) or a pharmaceutical formulation thereof, to a subject suffering from a C5-associated disease (e.g., PNH, aHUS, MG or CHAPLE), comprising introducing, into the body of the subject, one or more doses of about 30 mg/kg of antagonist antigen-binding protein that binds specifically to C5intravenously; and, optionally, one or more doses of the antagonist antigen-binding protein that binds specifically to C5 or a pharmaceutical formulation thereof subcutaneously.

The present invention also provides a dosing regimen for administering an antagonist antigen-binding protein that binds specifically to C5 (e.g., REGN3918) to a subject (e.g., a human) comprising introducing, into the body of the subject, (i) one or more doses of about 30 mg/kg of the anti-C5 antigen-binding protein intravenously (IV), then (ii) one or more doses of about 800 mg of the anti-C5 antigen-binding protein, subcutaneously (SC) (e.g., given weekly starting about 7 days after the first dose); or (a) one or more doses of about 30 mg/kg of the anti-C5 antigen-binding protein intravenously (IV), then (b) one or more subcutaneous doses (e.g., given weekly starting about 7 days after the first dose) based on body weight as follows: for body weight (BW)<10 kg, 125 mg, for BW ≥10 kg and <20 kg, 200 mg, for BW ≥20 kg and <40 kg, 350 mg, for BW ≥40 kg and <60 kg, 500 mg, and for BW ≥60 kg, 800 mg. For example, in an embodiment of the invention, the subcutaneous doses are administered once a week (weekly, q1w or qw). Weekly doses are, in an embodiment of the invention, administered about every 7 days, 7 days (±1 day), 7 days (±2 days) or 7 days (±3 days) after the immediately preceding dose. For example, if an initial dose is given on day 1, then a following weekly dose is given on about day 8 and about every 7 days thereafter. In an embodiment of the invention, the subject suffers from a C5-associated disease (e.g., PNH, CHAPLE, aHUS or MG).

The present invention further provides a method for treating or preventing CHAPLE disease, in a subject, by administering an antagonist antigen-binding protein that binds specifically to C5 set forth herein, e.g., REGN3918, to a subject, a therapeutically effective amount of the antigen-binding protein.

The present invention also provides a method for treating or preventing a C5-associated disease or reducing C5 complement activity (e.g., by about 99 or 100%, e.g., as measured by CH50 assay, e.g., CH50 assay measuring lysis of sheep red blood cells), in a subject (e.g., a human subject), comprising administering an antagonist antigen-binding protein that binds specifically to C5 (e.g., REGN3918), to the subject, by a dosing regimen discussed herein. In an embodiment of the invention, the C5-associated disease is adult respiratory distress syndrome; age-related macular degeneration (AMD); allergy; Alport's syndrome; Alzheimer's disease; amyotrophic lateral sclerosis (ALS); antiphospholipid syndrome (APS); asthma; atherosclerosis; atypical hemolytic uremic syndrome (aHUS); an autoimmune disease; autoimmune hemolytic anemia (AIHA); balloon angioplasty; bronchoconstriction; bullous pemphigoid; burns; C3 glomerulopathy; capillary leak syndrome; a cardiovascular disorder; catastrophic antiphospholipid syndrome (CAPS); a cerebrovascular disorder; CHAPLE disease (CD55 deficiency with hyperactivation of complement, angiopathic thrombosis, and protein-losing enteropathy); a chemical injury; chronic obstructive pulmonary disease (COPD); cold agglutinin disease (CAD); corneal and/or retinal tissue; Crohn's disease; Degos disease; dense deposit disease (DDD); dermatomyositis; diabetes; diabetic angiopathy; diabetic macular edema (DME); diabetic nephropathy; diabetic retinopathy; dilated cardiomyopathy; disorder of inappropriate or undesirable complement activation; dyspnea; eclampsia; emphysema; epidermolysis bullosa; epilepsy; fibrogenic dust disease; frostbite; geographic atrophy (GA); glomerulonephritis; glomerulopathy; Goodpasture's Syndrome; Graves' disease; Guillain-Barre Syndrome; Hashimoto's thyroiditis; hemodialysis complications; hemolysis-elevated liver enzymes- and low platelets (HELLP) syndrome; hemolytic anemia; hemoptysis; Henoch-Schonlein purpura nephritis; hereditary angioedema; hyperacute allograft rejection; hypersensitivity pneumonitis; idiopathic thrombocytopenic purpura (ITP); IgA nephropathy; an immune complex disorder; immune complex vasculitis; immune complex-associated inflammation; an infectious disease; inflammation caused by an autoimmune disease; an inflammatory disorder; inherited CD59 deficiency; injury due to inert dusts and/or minerals; interleukin-2 induced toxicity during IL-2 therapy; ischemia-reperfusion injury; Kawasaki's disease; a lung disease or disorder; lupus nephritis; membrane proliferative glomerulonephritis; membrano-proliferative nephritis; mesenteric artery reperfusion after aortic reconstruction; mesenteric/enteric vascular disorder; multifocal motor neuropathy (MMN); multiple sclerosis; myasthenia gravis; myocardial infarction; myocarditis; neurological disorder; neuromyelitis optica; obesity; ocular angiogenesis; ocular neovascularization affecting choroidal; organic dust disease; parasitic disease; Parkinson's disease; paroxysmal nocturnal hemoglobinuria (PNH); pauci-immune vasculitis; pemphigus; percutaneous transluminal coronary angioplasty (PTCA); peripheral (e.g., musculoskeletal) vascular disorder; pneumonia; post-ischemic reperfusion condition; post-pump syndrome in cardiopulmonary bypass; post-pump syndrome in renal bypass; pre-eclampsia; progressive kidney failure; proliferative nephritis; proteinuric kidney disease; psoriasis; pulmonary embolism; pulmonary fibrosis; pulmonary infarction; pulmonary vasculitis; recurrent fetal loss; a renal disorder; renal ischemia; renal ischemia-reperfusion injury; a renovascular disorder; restenosis following stent placement; rheumatoid arthritis (RA); rotational atherectomy; schizophrenia; sepsis; septic shock; SLE nephritis; smoke injury; spinal cord injury; spontaneous fetal loss; stroke; systemic inflammatory response to sepsis; systemic lupus erythematosus (SLE); systemic lupus erythematosus-associated vasculitis; Takayasu's disease; thermal injury; thrombotic thrombocytopenic purpura (TTP); traumatic brain injury; type I diabetes; typical hemolytic uremic syndrome (tHUS); uveitis; vasculitis; vasculitis associated with rheumatoid arthritis; venous gas embolus (VGE); and/or; xenograft rejection.

The present invention also provides a method for establishing and/or maintaining a concentration (e.g. a trough concentration) over time of at least about 100 mg/L, 150 mg/L, 400 mg/L, 600 mg/L, 700 mg/L or 600-700 mg/L of antagonist antigen-binding protein that binds specifically to C5 (e.g., REGN3918) in the serum of a subject (e.g., a human) and/or for achieving at least 80% (e.g., 81, 82, 93, 84, 85, 90, 95% or more) suppression of hemolysis in the serum of a subject (e.g., as measured by AH50 and/or CH50 assay) comprising administering the anti-C5 antigen-binding protein, to the subject, by a dosing regimen as discussed herein.

The present invention also provides a method for reducing serum lactate dehydrogenase (LDH) levels, intravascular hemolysis and/or the need for transfusions of red blood cells in a subject (e.g., a human) suffering from paroxysmal nocturnal hemoglobinuria (PNH) comprising administering the antagonist antigen-binding protein that binds specifically to C5 (e.g., REGN3918), to the subject, by a dosing regimen as discussed herein (e.g., (i) one or more doses of about 30 mg/kg of the antigen-binding protein intravenously (IV); then (ii) one or more weekly doses of about 800 mg of the antigen-binding protein, subcutaneously (SC).

In an embodiment of the invention, in a subject (e.g., suffering from PNH) receiving an antagonist antigen-binding protein that binds specifically to C5 as discussed herein (e.g., REGN3918), (i) the subject has a serum lactate dehydrogenase (LDH) level ≥2×upper limit of normal (ULN); (ii) the subject has PNH granulocytes (polymorphonuclear [PMN])) of >10%; (iii) the subject has hypoalbuminemia of less than or equal to 3.2 g/dL; (iv) the subject suffers from diarrhea; (v) the subject suffers from vomiting; (vi) the subject suffers from abdominal pain; (vii) the subject suffers from peripheral or facial edema; (viii) the subject suffers from an episode of infection with concomitant, hypogammaglobulinemia, or a thromboembolic event; (ix) the subject suffers from fatigue; (x) the subject suffers from hemoglobinuria; (xi) the subject suffers from shortness of breath (dyspnea); (xii) the subject suffers from anemia; (xiii) the subject suffers from a history of a major adverse vascular event; (xiv) the subject suffers from dysphagia; and/or (xv) the subject suffers from erectile dysfunction.

The present invention also provides a method for normalizing and/or increasing serum albumin or reducing therapeutic interventions, in a subject suffering from CD55-deficient protein-losing enteropathy, comprising administering an antagonist antigen-binding protein that binds specifically to C5 (e.g., REGN3918) to the subject by a method according to a dosing regimen set forth herein wherein the therapeutic intervention is one or more selected from the group consisting of: (i) administration of corticosteroids; (ii) administration of immunoglobulin; (iii) administration of albumin; (iv) administration of anti-tumor necrosis factor alpha therapeutic agent; (v) administration of immunomodulator; (vi) administration of micronutrient; (vii) administration of enteral or parenteral supplementation; (viii) administration of anti-coagulant; (ix) administration of antibiotic; and (x) administration of anti-platelet agent.

In an embodiment of the invention, in a subject (e.g., suffering from CHAPLE) receiving an antagonist antigen-binding protein that binds specifically to C5 as discussed herein (e.g., REGN3918), (i) the subject has a biallelic loss-of-function mutation in CD55; (ii) the subject has a biallelic loss-of-function mutation in CD55 which is a frame shift mutation; missense mutation, splice site mutation or nonsense mutation; (iii) the subject has hypoalbuminemia of less than or equal to 3.2 g/dL serum albumin; (iv) the subject suffers from diarrhea; (v) the subject suffers from vomiting; (vi) the subject suffers from abdominal pain; (vii) the subject suffers from peripheral or facial edema; (viii) the subject suffers from an episode of infection with concomitant hypogammaglobulinemia; and/or (ix) the subject suffers from a thrombotic event.

In an embodiment of the invention, an antagonist antigen-binding protein that binds specifically to C5 as discussed herein is an antibody or antigen-binding fragment thereof such as REGN3918 (pozelimab). In an embodiment of the invention, an antagonist antigen-binding protein that binds specifically to C5 as discussed herein (e.g., an antibody or antigen-binding fragment thereof) comprises (1) heavy chain variable region (HCVR) that comprises the amino acid sequence set forth in SEQ ID NO: 2 or HCDR1, HCDR2 and HCDR3 thereof, and a light chain variable region (LCVR) that comprises the amino acid sequence set forth in SEQ ID NO: 10 or LCDR1, LCDR2 and LCDR3 thereof or LCDR1, LCDR2 and LCDR3 thereof; (2) HCVR that comprises the amino acid sequence set forth in SEQ ID NO: 18 or HCDR1, HCDR2 and HCDR3 thereof, and an LCVR that comprises the amino acid sequence set forth in SEQ ID NO: 26 or LCDR1, LCDR2 and LCDR3 thereof; (3) HCVR that comprises the amino acid sequence set forth in SEQ ID NO: 34 or HCDR1, HCDR2 and HCDR3 thereof, and an LCVR that comprises the amino acid sequence set forth in SEQ ID NO: 42 or LCDR1, LCDR2 and LCDR3 thereof; (4) HCVR that comprises the amino acid sequence set forth in SEQ ID NO: 50 or HCDR1, HCDR2 and HCDR3 thereof, and an LCVR that comprises the amino acid sequence set forth in SEQ ID NO: 58 or LCDR1, LCDR2 and LCDR3 thereof; (5) HCVR that comprises the amino acid sequence set forth in SEQ ID NO: 66 or HCDR1, HCDR2 and HCDR3 thereof, and an LCVR that comprises the amino acid sequence set forth in SEQ ID NO: 74 or LCDR1, LCDR2 and LCDR3 thereof; (6) HCVR that comprises the amino acid sequence set forth in SEQ ID NO: 82 or HCDR1, HCDR2 and HCDR3 thereof, and an LCVR that comprises the amino acid sequence set forth in SEQ ID NO: 90 or LCDR1, LCDR2 and LCDR3 thereof; (7) HCVR that comprises the amino acid sequence set forth in SEQ ID NO: 98 or HCDR1, HCDR2 and HCDR3 thereof, and an LCVR that comprises the amino acid sequence set forth in SEQ ID NO: 106 or LCDR1, LCDR2 and LCDR3 thereof; (8) HCVR that comprises the amino acid sequence set forth in SEQ ID NO: 98 or HCDR1, HCDR2 and HCDR3 thereof, and an LCVR that comprises the amino acid sequence set forth in SEQ ID NO: 114 or LCDR1, LCDR2 and LCDR3 thereof; (9) HCVR that comprises the amino acid sequence set forth in SEQ ID NO: 122 or HCDR1, HCDR2 and HCDR3 thereof, and an LCVR that comprises the amino acid sequence set forth in SEQ ID NO: 106 or LCDR1, LCDR2 and LCDR3 thereof; (10) HCVR that comprises the amino acid sequence set forth in SEQ ID NO: 98 or HCDR1, HCDR2 and HCDR3 thereof, and an LCVR that comprises the amino acid sequence set forth in SEQ ID NO: 130 or LCDR1, LCDR2 and LCDR3 thereof; (11) HCVR that comprises the amino acid sequence set forth in SEQ ID NO: 138 or HCDR1, HCDR2 and HCDR3 thereof, and an LCVR that comprises the amino acid sequence set forth in SEQ ID NO: 106 or LCDR1, LCDR2 and LCDR3 thereof; (12) HCVR that comprises the amino acid sequence set forth in SEQ ID NO: 146 or HCDR1, HCDR2 and HCDR3 thereof, and an LCVR that comprises the amino acid sequence set forth in SEQ ID NO: 106 or LCDR1, LCDR2 and LCDR3 thereof; (13) HCVR that comprises the amino acid sequence set forth in SEQ ID NO: 122 or HCDR1, HCDR2 and HCDR3 thereof, and an LCVR that comprises the amino acid sequence set forth in SEQ ID NO: 130 or LCDR1, LCDR2 and LCDR3 thereof; (14) HCVR that comprises the amino acid sequence set forth in SEQ ID NO: 146 or HCDR1, HCDR2 and HCDR3 thereof, and an LCVR that comprises the amino acid sequence set forth in SEQ ID NO: 114 or LCDR1, LCDR2 and LCDR3 thereof; (15) HCVR that comprises the amino acid sequence set forth in SEQ ID NO: 146 or HCDR1, HCDR2 and HCDR3 thereof, and an LCVR that comprises the amino acid sequence set forth in SEQ ID NO: 130 or LCDR1, LCDR2 and LCDR3 thereof; (16) HCVR that comprises the amino acid sequence set forth in SEQ ID NO: 138 or HCDR1, HCDR2 and HCDR3 thereof, and an LCVR that comprises the amino acid sequence set forth in SEQ ID NO: 130 or LCDR1, LCDR2 and LCDR3 thereof; (17) HCVR that comprises the amino acid sequence set forth in SEQ ID NO: 154 or HCDR1, HCDR2 and HCDR3 thereof, and an LCVR that comprises the amino acid sequence set forth in SEQ ID NO: 162 or LCDR1, LCDR2 and LCDR3 thereof; (18) HCVR that comprises the amino acid sequence set forth in SEQ ID NO: 170 or HCDR1, HCDR2 and HCDR3 thereof, and an LCVR that comprises the amino acid sequence set forth in SEQ ID NO: 178 or LCDR1, LCDR2 and LCDR3 thereof; (19) HCVR that comprises the amino acid sequence set forth in SEQ ID NO: 186 or HCDR1, HCDR2 and HCDR3 thereof, and an LCVR that comprises the amino acid sequence set forth in SEQ ID NO: 194 or LCDR1, LCDR2 and LCDR3 thereof; (20) HCVR that comprises the amino acid sequence set forth in SEQ ID NO: 202 or HCDR1, HCDR2 and HCDR3 thereof, and an LCVR that comprises the amino acid sequence set forth in SEQ ID NO: 210 or LCDR1, LCDR2 and LCDR3 thereof; (21) HCVR that comprises the amino acid sequence set forth in SEQ ID NO: 218 or HCDR1, HCDR2 and HCDR3 thereof, and an LCVR that comprises the amino acid sequence set forth in SEQ ID NO: 226 or LCDR1, LCDR2 and LCDR3 thereof; (22) HCVR that comprises the amino acid sequence set forth in SEQ ID NO: 234 or HCDR1, HCDR2 and HCDR3 thereof, and an LCVR that comprises the amino acid sequence set forth in SEQ ID NO: 242 or LCDR1, LCDR2 and LCDR3 thereof; (23) HCVR that comprises the amino acid sequence set forth in SEQ ID NO: 250 or HCDR1, HCDR2 and HCDR3 thereof, and an LCVR that comprises the amino acid sequence set forth in SEQ ID NO: 258 or LCDR1, LCDR2 and LCDR3 thereof; (24) HCVR that comprises the amino acid sequence set forth in SEQ ID NO: 266 or HCDR1, HCDR2 and HCDR3 thereof, and an LCVR that comprises the amino acid sequence set forth in SEQ ID NO: 258 or LCDR1, LCDR2 and LCDR3 thereof; (25) HCVR that comprises the amino acid sequence set forth in SEQ ID NO: 274 or HCDR1, HCDR2 and HCDR3 thereof, and an LCVR that comprises the amino acid sequence set forth in SEQ ID NO: 282 or LCDR1, LCDR2 and LCDR3 thereof; (26) HCVR that comprises the amino acid sequence set forth in SEQ ID NO: 290 or HCDR1, HCDR2 and HCDR3 thereof, and an LCVR that comprises the amino acid sequence set forth in SEQ ID NO: 298 or LCDR1, LCDR2 and LCDR3 thereof; (27) HCVR that comprises the amino acid sequence set forth in SEQ ID NO: 306 or HCDR1, HCDR2 and HCDR3 thereof, and an LCVR that comprises the amino acid sequence set forth in SEQ ID NO: 314 or LCDR1, LCDR2 and LCDR3 thereof; (28) HCVR that comprises the amino acid sequence set forth in SEQ ID NO: 322 or HCDR1, HCDR2 and HCDR3 thereof, and an LCVR that comprises the amino acid sequence set forth in SEQ ID NO: 330 or LCDR1, LCDR2 and LCDR3 thereof; and/or, (29) HCVR that comprises the amino acid sequence set forth in SEQ ID NO: 338 or HCDR1, HCDR2 and HCDR3 thereof, and an LCVR that comprises the amino acid sequence set forth in SEQ ID NO: 346 or LCDR1, LCDR2 and LCDR3 thereof; or competes for binding to C5 with an antigen-binding protein selected from the group consisting of (1)-(29); or binds to the same epitope on C5 as an antigen-binding protein selected from the group consisting of (1)-(29). In an embodiment of the invention, a subject receiving an antagonist antigen-binding protein that binds specifically to C5 as set forth herein has previously received tesidolumab, eculizumab or ravulizumab. In an embodiment of the invention, an antagonist antigen-binding protein that binds specifically to C5 as set forth herein is administered in association with a further therapeutic agent; e.g., cemdisiran, an oligonucleotide, anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, or methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor, an anti-thrombotic agent, an antibiotic, penicillin, erythromycin, a vaccine, a Meningococcal vaccine, an anti-fungal agent, an anti-viral agent, a corticosteroid, erythropoietin, an immunosuppressive drug, an anti-coagulant, an iron supplement, folic acid, acetaminophen, aspirin, ibuprofen or hormone-replacement therapy. In an embodiment of the invention, the further therapeutic agent is an oligonucleotide which is a DNA oligonucleotide, an RNA oligonucleotide, a single stranded DNA oligonucleotide, a single stranded RNA oligonucleotide, a double stranded DNA oligonucleotide, or a double stranded RNA oligonucleotide; optionally, wherein the oligonucleotide is conjugated to a sugar.

The present invention also provides a dosing regimen for administering an antagonist antigen-binding protein that binds specifically to C5 (e.g., REGN3918) to a subject (e.g., a human) comprising introducing, into the body of the subject,

(i) one or more doses of about 30 mg/kg of the anti-C5 antigen-binding protein intravenously (IV), and/or
(ii) one or more doses of about 800 mg of the anti-C5 antigen-binding protein, subcutaneously (SC) (e.g., given weekly starting about 7 days after the first dose); or
(a) one or more doses of about 30 mg/kg of the anti-C5 antigen-binding protein intravenously (IV), and/or
(b) one or more subcutaneous doses (e.g., given weekly starting about 7 days after the first dose) based on body weight as follows: for body weight (BW)<10 kg, 125 mg, for BW ≥10 kg and <20 kg, 200 mg, for BW ≥20 kg and <40 kg, 350 mg, for BW ≥40 kg and <60 kg, 500 mg, and for BW ≥60 kg, 800 mg. As set forth above, (i) and (ii) can be in either order and (a) and (b) can be in either order. For example, in an embodiment of the invention, the subcutaneous doses are administered once a week (weekly, q1w or qw). Weekly doses are, in an embodiment of the invention, administered about every 7 days, 7 days (±1 day), 7 days (±2 days) or 7 days (±3 days) after the immediately preceding dose. For example, if an initial dose is given on day 1, then a following weekly dose is given on about day 8 and about every 7 days thereafter. In an embodiment of the invention, the subject suffers from a C5-associated disease (e.g., PNH, CHAPLE, aHUS or MG). Methods for treating or preventing a C5-associated disorder comprising said administration methods are within the scope of the present invention.

### BRIEF DESCRIPTION OF THE FIGURES

FIG. 1. Illustration of cohorts in Study of REGN3918 in Patients with paroxysmal nocturnal hemoglobinuria (PNH).

FIG. 2. Mean (±SE) serum concentrations of total REGN3918 versus nominal time in each treatment group (1 mg/kg IV, single dose; 3 mg/kg IV, single dose; 300 mg SC, single dose; 10 mg/kg IV, single dose; 600 mg SC, single dose; 30 mg/kg IV, single dose; or 15 mg/kg IV followed by 4 repeat SC doses of 400 mg q1w×4 weeks) in healthy human volunteers.

FIG. 3. Mean (±SE) percentage change from baseline in CH50 versus nominal time in each treatment group (1 mg/kg IV, single dose; 3 mg/kg IV, single dose; 300 mg SC, single dose; 10 mg/kg IV, single dose; 600 mg SC, single dose; 30 mg/kg IV, single dose; or 15 mg/kg IV followed by 4 repeat SC doses of 400 mg q1w×4 weeks) in healthy human volunteers.

FIG. 4 (A-I). In vitro alternative pathway (AP) and classical pathway (CP) hemolysis in the presence of various concentrations of pozelimab (REGN3918), eculizumab, ravulizumab or isotype control antibody (REGN1945). FIG. 4A shows AP hemolysis assay in the presence of 10% normal human serum (NHS). FIG. 4B shows AP hemolysis assay in the presence of 25% NHS. FIG. 4C shows AP hemolysis assay in the presence of 48% NHS. FIG. 4D shows CP hemolysis assays in the presence of 5% NHS. FIG. 4E shows CP hemolysis assays in the presence of 10% NHS. FIG. 4F shows CP hemolysis assays in the presence of 25% NHS. FIG. 4G shows AP hemolysis assay in the presence of 25% NHS and 1 mM MgCl₂. FIG. 4H shows AP hemolysis assay in the presence of 25% NHS and 1.5 mM MgCl₂. FIG. 4I shows AP hemolysis assay in the presence of 25% NHS and 2 mM MgCl₂.

FIG. 5. Lactate dehydrogenase (LDH) (X ULN) for six patients (410001001F; 410001002F; 410004001F; 410004002M; 410005001F and 410005002M) over time in normal scale. The LDH upper limit of normal (ULN) and 1.5×ULN is indicated.

FIG. 6. Lactate dehydrogenase (LDH) (X ULN), in semi-log scale, for six patients over time. The LDH upper limit of normal (ULN) and 1.5×ULN is indicated.

FIG. 7. Mean lactate dehydrogenase (LDH) (X ULN) for six patients over time in normal scale. The LDH upper limit of normal (ULN) and 1.5×ULN is indicated.

FIG. 8. Mean lactate dehydrogenase (LDH) (X ULN), in semi-log scale, for six patients over time. The LDH upper limit of normal (ULN) and 1.5×ULN is indicated.

FIG. 9 (A-D). FIG. 9A shows individual, normal scale concentration of total REGN3918 in serum (mg/L) versus nominal time in first 6 PNH naïve patients. FIG. 9B shows individual, semi-log scale concentration of total REGN3918 in serum (mg/L) versus nominal time in first 6 PNH naïve patients. FIG. 9C shows median, normal scale concentration of total REGN3918 in serum (mg/L) versus nominal time in first 6 PNH naïve patients. FIG. 9D shows median, semi-log scale concentration of total REGN3918 in serum (mg/L) versus nominal time in first 6 PNH naïve patients.

FIG. 10 (A-D). FIG. 10A shows individual, normal scale concentration of total REGN3918 in serum (mg/L) versus nominal time in first 6 PNH naïve patients, by gender. FIG. 10B shows individual, semi-log scale concentration of total REGN3918 in serum (mg/L) versus nominal time in first 6 PNH naïve patients, by gender. FIG. 10C shows median, normal scale concentration of total REGN3918 in serum (mg/L) versus nominal time in first 6 PNH naïve patients, by gender. FIG. 10D shows median, semi-log scale concentration of total REGN3918 in serum (mg/L) versus nominal time in first 6 PNH naïve patients, by gender.

FIG. 11 (A-B). FIG. 11A shows individual concentration of total C5 in plasma (mg/L) versus nominal time in first 6 PNH naïve patients. FIG. 11B shows median concentration of total C5 in plasma (mg/L) versus nominal time in first 6 PNH naïve patients.

FIG. 12 (A-B). FIG. 12A shows individual concentration of total C5 in plasma (mg/L) versus nominal time in first 6 PNH naïve patients, by gender. FIG. 12B shows median concentration of total C5 in plasma (mg/L) versus nominal time in first 6 PNH naïve patients, by gender.

FIG. 13 (A-B). FIG. 13A shows individual fold over baseline total C5 in plasma by nominal time in first 6 PNH naïve patients. FIG. 13B shows median fold over baseline total C5 in plasma by nominal time in first 6 PNH naïve patients.

FIG. 14 (A-B). FIG. 14A shows individual fold over baseline total C5 in plasma by nominal time in first 6 PNH naïve patients, by gender. FIG. 14B shows median fold over baseline total C5 in plasma by nominal time in first 6 PNH naïve patients, by gender.

FIG. 15. Mean (±SD) concentration (mg/L) of total C5 in plasma versus nominal time in first 6 PNH naïve patients.

FIG. 16. Collection of graphs showing individual concentrations, in six patients (A, B, C, D, E, and F), of total REGN3918 (TOR3918; triangles) and total C5 (TOC5; circles) versus nominal time.

FIG. 17. LDH (X ULN) for 6 patients beyond day 57 (Female LDH ULN=330 U/L and Male LDH ULN=281 U/L).

FIG. 18. Semi-log scale LDH (X ULN) for 6 patients beyond day 57 (Female LDH ULN=330 U/L and Male LDH ULN=281 U/L).

FIG. 19. Mean LDH (X ULN) for 6 patients beyond day 57 (Female LDH ULN=330 U/L and Male LDH ULN=281 U/L).

FIG. 20. Semi-log scale mean LDH (X ULN) for 6 patients beyond day 57 (Female LDH ULN=330 U/L and Male LDH ULN=281 U/L).

FIG. 21. LDH (X ULN) for 9 patients beyond day 57 (Female LDH ULN=330 U/L and Male LDH ULN=281 U/L).

FIG. 22. Semi-log scale LDH (X ULN) for 9 patients beyond day 57 (Female LDH ULN=330 U/L and Male LDH ULN=281 U/L).

FIG. 23. Mean LDH (X ULN) for 9 patients beyond day 57 (Female LDH ULN=330 U/L and Male LDH ULN=281 U/L).

FIG. 24. Semi-log scale mean LDH (X ULN) for 9 patients beyond day 57.

FIG. 25. Summary of study of treatment of naïve PNH patients with ALXN1210 (ravulizumab) or eculizumab.

FIG. 26 (A-D). Dose switching from eculizumab to REGN3918 results in normalization of serum C5 concentrations and maintained suppression of hemolytic activity.

FIG. 26A shows total hIgG concentration was measured by Gyros in serum collected from C5hu/hu mice administered 3 doses of REGN3918 alone (closed circles), 3 doses of eculizumab alone (squares), or 1 dose of eculizumab followed by 2 doses of REGN3918 (switch, open circles). Arrowheads on y-axis and vertical grey dashed lines indicate times of dosing. FIG. 26B shows total C5 serum concentrations in C5hu/hu mice administered REGN3918 alone (closed circles), eculizumab alone (squares), or switched from eculizumab to REGN3918 (switch, open circles) were measured from mice bled over the duration of the study. FIG. 26C shows serum collected from terminally bled C5hu/hu mice administered with REGN3918 alone (closed circles), eculizumab alone (squares), or eculizumab/REGN3918 switched (open circles) were supplemented with hC3 and the percent of CP-mediated hemolysis using ex vivo assays was assessed. FIG. 26D shows serum concentrations of total C5 and hIgG were used to calculate the ratio of C5:mAb at the indicated times. Data is plotted as mean±SEM.

FIG. 27. REGN3918 and eculizumab bind to distinct sites on C5 and when all three are present under conditions that are designed to mimic dose switching, form complexes predominantly containing 1 to 2 molecules of C5. Eculizumab:C5 complexes were analyzed by asymmetric flow field-flow fractionation coupled to multi-angle laser light scattering (A4F-MALLS). Fractograms from individual samples of eculizumab, C5, and REGN3918 are also overlaid. Relative UV absorbance at 215 nm as a function of retention time is shown for each sample and the measured molar masses of resolved peaks are indicated.

FIG. 28 (A-E). Plots of serum albumin levels in four individual CHAPLE patients over time. FIG. 28A shows serum albumin levels of the four CHAPLE patients during the treatment period. FIG. 28B shows serum albumin levels of a first of the four CHAPLE patients before treatment. FIG. 28C shows serum albumin levels of a second of the four CHAPLE patients before treatment. FIG. 28D shows serum albumin levels of a third of the four CHAPLE patients before treatment. FIG. 28E shows serum albumin levels of a fourth of the four CHAPLE patients before treatment. The lower level of normal (LLN) for the male and female patients is indicated.

FIG. 29. Plot of total serum protein in four individual CHAPLE patients over time starting at baseline. The lower level of normal (LLN) and upper level of normal (ULN) are indicated.

FIG. 30. Plot of vitamin B12 levels over time in four individual CHAPLE patients during the treatment period from baseline.

FIG. 31. Plot of platelet counts over time in four individual CHAPLE patients during the treatment period from baseline.

FIG. 32. Plot of fecal alpha-1-antitrypsin concentration over time in four individual CHAPLE patients during the treatment period from baseline. The upper level of normal

(ULN) is indicated.

FIG. 33. Plot of facial edema grade in four individual CHAPLE patients over time before and during the treatment period.

FIG. 34. Plot of peripheral edema grade in four individual CHAPLE patients over time before and during the treatment period.

FIG. 35. Plot of the daily average of bowel movements per week for four individual CHAPLE patients.

### DETAILED DESCRIPTION OF THE INVENTION

A convenient REGN3918 (pozelimab) dosing regimen, including a subcutaneous dosing component, for treating C5-related disorders, such as PNH, in humans has been developed. Subcutaneous dosing gives patients the option for home-dosing and, thus, offers the advantage of greater patient compliance over the IV dosing regimens of eculizumab and ravulizumab. This dosing regimen has been shown to be highly effective for controlling hemolysis and reducing breakthrough hemolysis in human patients receiving the antibody. REGN3918 administered 30 mg/kg IV followed by 800 mg once weekly SC (REGN3918-30+800 dosing regimen) demonstrated robust inhibition of intravascular hemolysis, with normalization of LDH in human patients with PNH. Though REGN3918 is known to bind with high affinity to C5 (R885H/C), it has been shown, in human PNH patients receiving the REGN3918-30+800 dosing regimen, to effectively normalize LDH. Data presented herein demonstrated REGN3918-30+800 dosing regimen efficacy in a human patient with the C5 variant that was resistant to prior eculizumab therapy. The REGN3918-30+800 dosing regimen also exhibited clinical advantages over eculizumab and ravulizumab. Treatment with REGN3918 led to a rapid, robust, and sustained reduction of LDH through study day 57; and LDH in all 6 patients was reduced at day 3 (48 h after one dose), with the achievement of control of intravascular hemolysis, with LDH ≤1.5×ULN (upper limit of normal), at day 14, and with normalization of LDH (≤1.0×ULN) at day 29. In contrast, evidence suggests that, in patients receiving ravulizumab and eculizumab, only about half achieve LDH normalization. Indeed, 25% of PNH patients receiving eculizumab still need recurrent, albeit less frequent, blood transfusions; and up to 20% of the patients require significant increases in dose or dose frequency due to breakthrough hemolysis secondary to incomplete inhibition of C5. See Nakayama et al., Eculizumab Dosing Intervals Longer than 17 Days May Be Associated with Greater Risk of Breakthrough Hemolysis in Patients with Paroxysmal Nocturnal Hemoglobinuria. Biol Pharm Bull 2016; 39(2):285-8; Hil et al., Thrombosis in paroxysmal nocturnal hemoglobinuria. Blood 2013; 121(25):4985-96; and Peffault de Latour et al., Assessing complement blockade in patients with paroxysmal nocturnal hemoglobinuria receiving eculizumab. Blood 2015; 125(5):775-83. Comparative ex vivo hemolysis assays presented herein suggest that pozelimab was more effective than ravulizumab and eculizumab at inhibiting AP complement-mediated hemolysis and better than ravulizumab at inhibiting CP complement-mediated hemolysis.

A discussion of a dosing regimen comprising administering A and then, optionally, B refers to regimens comprising administering only A as well as regimens comprising administering A and then B.

### Antagonist Antigen-Binding Protein that Binds Specifically to C5

The present invention provides methods for using antagonist antigen-binding proteins that bind specifically to C5 (e.g., antibodies and antigen-binding fragments thereof) and pharmaceutical formulations thereof comprising a pharmaceutically acceptable carrier, as specified herein.

In an embodiment of the invention, the antagonist antigen-binding protein that binds specifically to C5 binds to the beta chain or the alpha chain of C5 or both, e.g., at residues 591-599 and/or 775-794, e.g., NMATGMDSW (SEQ ID NO: 353) and/or WEVHLVPRRKQLQFALPDSL (SEQ ID NO: 354). In an embodiment of the invention, the anti-C5 antigen-binding protein does not bind C5a.

In an embodiment of the invention, the antagonist antigen-binding protein that binds specifically to C5 binds at residues KDMQLGRLHMKTLLPVSK (SEQ ID NO: 355).

In an embodiment of the invention, the antagonist antigen-binding protein that binds specifically to C5 binds the beta chain of C5 thereof, e.g. at residues 332-398, 332-378, 332-364, 332-348, 350-420, 369-409, 379-398 and/or 386-392.

In an embodiment of the invention, the antagonist antigen-binding protein that binds specifically to C5 binds C5a, e.g. at residues NDETCEQRA (SEQ ID NO: 356) and/or SHKDMQL (SEQ ID NO: 357).

In an embodiment of the invention, the antagonist antigen-binding protein that binds specifically to C5 binds the beta chain of C5, e.g., residues 19-180. In an embodiment of the invention, binding to C5 is reduced by E48A, D51A and/or K109A C5 mutations.

Immunoglobulin polypeptides in antagonist antigen-binding protein that binds specifically to C5 (e.g., antibody or antigen-binding fragment thereof) which may be used in the methods of the present invention are set forth in Table A.

TABLE A
Anti-C5 Antibody Chain Amino Acid Sequences*

| Antibody | SEQ ID NOs | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 2 | 4 | 6 | 8 | 10 | 12 | 14 | 16 |
| H2M11686N | 18 | 20 | 22 | 24 | 26 | 28 | 30 | 32 |
| H4H12159P | 34 | 36 | 38 | 40 | 42 | 44 | 46 | 48 |
| H4H12161P | 50 | 52 | 54 | 56 | 58 | 60 | 62 | 64 |
| H4H12163P | 66 | 68 | 70 | 72 | 74 | 76 | 78 | 80 |
| H4H12164P | 82 | 84 | 86 | 88 | 90 | 92 | 94 | 96 |
| H4H12166P | 98 | 100 | 102 | 104 | 106 | 108 | 110 | 112 |
| H4H12166P2 | 98 | 100 | 102 | 104 | 114 | 116 | 118 | 120 |
| H4H12166P3 | 122 | 124 | 126 | 128 | 106 | 108 | 110 | 112 |
| H4H12166P4 | 98 | 100 | 102 | 104 | 130 | 132 | 134 | 136 |
| H4H12166P5 | 138 | 140 | 142 | 144 | 106 | 108 | 110 | 112 |
| H4H12166P6 | 146 | 148 | 150 | 152 | 106 | 108 | 110 | 112 |
| H4H12166P7 | 122 | 124 | 126 | 128 | 130 | 132 | 134 | 136 |
| H4H12166P8 | 146 | 148 | 150 | 152 | 114 | 116 | 118 | 120 |
| H4H12166P9 | 146 | 148 | 150 | 152 | 130 | 132 | 134 | 136 |
| H4H12166P10 | 138 | 140 | 142 | 144 | 130 | 132 | 134 | 136 |
| H4H12167P | 154 | 156 | 158 | 160 | 162 | 164 | 166 | 168 |
| H4H12168P | 170 | 172 | 174 | 176 | 178 | 180 | 182 | 184 |
| H4H12169P | 186 | 188 | 190 | 192 | 194 | 196 | 198 | 200 |
| H4H12170P | 202 | 204 | 206 | 208 | 210 | 212 | 214 | 216 |
| H4H12171P | 218 | 220 | 222 | 224 | 226 | 228 | 230 | 232 |
| H4H12175P | 234 | 236 | 238 | 240 | 242 | 244 | 246 | 248 |
| H4H12176P2 | 250 | 252 | 254 | 256 | 258 | 260 | 262 | 264 |
| H4H12177P2 | 266 | 268 | 270 | 272 | 258 | 260 | 262 | 264 |
| H4H12183P2 | 274 | 276 | 278 | 280 | 282 | 284 | 286 | 288 |
| H2M11682N | 290 | 292 | 294 | 296 | 298 | 300 | 302 | 304 |
| H2M11684N | 306 | 308 | 310 | 312 | 314 | 316 | 318 | 320 |
| H2M11694N | 322 | 324 | 326 | 328 | 330 | 332 | 334 | 336 |
| H2M11695N | 338 | 340 | 342 | 344 | 346 | 348 | 350 | 352 |

*Antibodies and fragments may include one or more variants of said sequences See WO2017/218515

Polynucleotides encoding the chains set forth in Table A are set forth below in Table B.

TABLE B
Anti-C5 Antibody Chain Nucleotide Sequences*

| Antibody | SEQ ID NOs | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 1 | 3 | 5 | 7 | 9 | 11 | 13 | 15 |
| H2M11686N | 17 | 19 | 21 | 23 | 25 | 27 | 29 | 31 |
| H4H12159P | 33 | 35 | 37 | 39 | 41 | 43 | 45 | 47 |
| H4H12161P | 49 | 51 | 53 | 55 | 57 | 59 | 61 | 63 |
| H4H12163P | 65 | 67 | 69 | 71 | 73 | 75 | 77 | 79 |
| H4H12164P | 81 | 83 | 85 | 87 | 89 | 91 | 93 | 95 |
| H4H12166P | 97 | 99 | 101 | 103 | 105 | 107 | 109 | 111 |
| H4H12166P2 | 97 | 99 | 101 | 103 | 113 | 115 | 117 | 119 |
| H4H12166P3 | 121 | 123 | 125 | 127 | 105 | 107 | 109 | 111 |
| H4H12166P4 | 97 | 99 | 101 | 103 | 129 | 131 | 133 | 135 |
| H4H12166P5 | 137 | 139 | 141 | 143 | 105 | 107 | 109 | 111 |
| H4H12166P6 | 145 | 147 | 149 | 151 | 105 | 107 | 109 | 111 |
| H4H12166P7 | 121 | 123 | 125 | 127 | 129 | 131 | 133 | 135 |
| H4H12166P8 | 145 | 147 | 149 | 151 | 113 | 115 | 117 | 119 |
| H4H12166P9 | 145 | 147 | 149 | 151 | 129 | 131 | 133 | 135 |
| H4H12166P10 | 137 | 139 | 141 | 143 | 129 | 131 | 133 | 135 |
| H4H12167P | 153 | 155 | 157 | 159 | 161 | 163 | 165 | 167 |
| H4H12168P | 169 | 171 | 173 | 175 | 177 | 179 | 181 | 183 |
| H4H12169P | 185 | 187 | 189 | 191 | 193 | 195 | 197 | 199 |
| H4H12170P | 201 | 203 | 205 | 207 | 209 | 211 | 213 | 215 |
| H4H12171P | 217 | 219 | 221 | 223 | 225 | 227 | 229 | 231 |
| H4H12175P | 233 | 235 | 237 | 239 | 241 | 243 | 245 | 247 |
| H4H12176P2 | 249 | 251 | 253 | 255 | 257 | 259 | 261 | 263 |
| H4H12177P2 | 265 | 267 | 269 | 271 | 257 | 259 | 261 | 263 |
| H4H12183P2 | 273 | 275 | 277 | 279 | 281 | 283 | 285 | 287 |
| H2M11682N | 289 | 291 | 293 | 295 | 297 | 299 | 301 | 303 |
| H2M11684N | 305 | 307 | 309 | 311 | 313 | 315 | 317 | 319 |
| H2M11694N | 321 | 323 | 325 | 327 | 329 | 331 | 333 | 335 |
| H2M11695N | 337 | 339 | 341 | 343 | 345 | 347 | 349 | 351 |

*Antibodies and fragments may include one or more variants of said sequences See WO2017/218515

```
H2M11683N
HCVR
(SEQ ID NO: 2)
Gln Val Gln Leu Val Glu Ser Gly Gly Gly Val Val Gln Pro Gly Arg
1 5 10 15
Ser Leu Arg Leu Ser Cys Val Ala Ser Gly Phe Thr Phe Ser Ser Tyr
              20 25 30
Gly Ile His Trp Val Arg Gln Ala Pro Gly Lys Gly Leu Glu Trp Val
            35 40 45
Ala Val Ile Trp Asp Asp Gly Asn Asn Ile Asn Tyr Ser Asp Ser Val
    50 55 60
Lys Gly Arg Phe Ile Ile Ser Arg Asp Asn Ser Arg Lys Thr Val Tyr
65 70 75 80
Leu Gln Met Asn Ser Leu Arg Gly Glu Asp Thr Ala Val Tyr Tyr Cys
                  85 90 95
Ala Arg Asp Ala Pro Ile Ala Pro Val Pro Asp Tyr Trp Gly Gln Gly
                100 105 110
Thr Leu Val Thr Val Ser Ser
                    115
LCVR
(SEQ ID NO: 10)
Asp Ile Gln Met Thr Gln Ser Pro Ser Thr Leu Ser Ala Ser Val Gly
1 5 10 15
Asp Arg Val Thr Ile Thr Cys Arg Ala Ser Gln Ser Ile Ser Ser Trp
              20 25 30
Leu Ala Trp Tyr Gln Gln Lys Pro Gly Lys Ala Pro Lys Leu Leu Ile
            35 40 45
Tyr Lys Ala Ser Ser Leu Asp Thr Gly Val Pro Ser Arg Phe Ser Gly
    50 55 60
Ser Gly Ser Gly Thr Glu Phe Thr Leu Thr Ile Ser Ser Leu Gln Pro
65 70 75 80
Asp Asp Phe Ala Thr Tyr Tyr Cys Gln Gln Tyr Asn Thr Tyr Ser Tyr
                  85 90 95
Thr Phe Gly Leu Gly Thr Lys Leu Glu Ile Lys
                100 105
H2M11686N
HCVR
(SEQ ID NO: 18)
Gln Val Gln Leu Val Glu Ser Gly Gly Gly Leu Val Lys Pro Gly Gly
1 5 10 15
Ser Leu Arg Leu Ser Cys Ala Ala Ser Gly Phe Thr Phe Ser Asp Tyr
              20 25 30
Tyr Met Ser Trp Ile Arg Gln Ala Pro Gly Lys Gly Leu Glu Trp Val
            35 40 45
Ser Tyr Ile Ser Ser Ser Gly Asn Thr Ile Lys Tyr Ala Asp Ser Met
    50 55 60
Lys Gly Arg Phe Thr Ile Ser Arg Asp Asn Ala Lys Lys Ser Leu Phe
65 70 75 80
Val Glu Met Asn Ser Leu Arg Ala Glu Asp Thr Ala Val Tyr Tyr Cys
                  85 90 95
Ala Arg Tyr Lys Ser Ser Ser Asp Tyr Phe Asp His Trp Gly Gln Gly
                100 105 110
Thr Leu Val Thr Val Ser Ser
          115
LCVR
(SEQ ID NO: 26)
Glu Ile Val Leu Thr Gln Ser Pro Ala Thr Leu Ser Leu Ser Pro Gly
1 5 10 15
Glu Arg Ala Thr Leu Ser Cys Arg Ala Ser Gln Ser Val Arg Ser Tyr
              20 25 30
Leu Ala Trp Tyr Gln Gln Lys Pro Gly Gln Ala Pro Arg Leu Leu Ile
            35 40 45
Tyr Asp Ala Ser Asn Arg Ala Thr Ala Ile Pro Ala Arg Phe Ser Gly
    50 55 60
Ser Gly Ser Gly Thr Asp Phe Thr Leu Thr Ile Ser Ser Leu Glu Pro
65 70 75 80
Glu Asp Leu Ala Val Tyr Tyr Cys Gln Gln Ser Gly Asn Trp Pro Leu
                  85 90 95
Thr Phe Gly Gly Gly Thr Lys Val Glu Ile Lys
                100 105
H4H12159P
HCVR
(SEQ ID NO: 34)
Gln Val Gln Leu Val Glu Ser Gly Gly Gly Val Val Gln Pro Gly Arg
1 5 10 15
Ser Leu Arg Leu Ser Cys Gly Ala Ser Gly Phe Thr Phe Ser Thr Tyr
              20 25 30
Gly Met His Trp Val Arg Gln Ala Pro Gly Lys Gly Leu Glu Trp Val
            35 40 45
Ala Val Ile Trp Asp Asp Gly Asn Asn Lys Tyr Tyr Ala Asp Ser Val
    50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
                  85 90 95
Ala Arg Asp Ser Glu Val Ala Pro Val Gly Asp Tyr Trp Gly Gln Gly
                100 105 110
Thr Leu Val Thr Val Ser Ser
          115
LCVR
(SEQ ID NO: 42)
Asp Ile Gln Met Thr Gln Ser Pro Ser Thr Leu Ser Ala Ser Val Gly
1 5 10 15
Asp Arg Val Thr Ile Ile Cys Arg Ala Ser Gln Ser Ile Asn Arg Trp
              20 25 30
Leu Ala Trp Tyr Gln Gln Lys Pro Gly Lys Ala Pro Lys Leu Leu Ile
            35 40 45
Tyr Lys Ala Ser Ser Leu Glu Ser Gly Val Pro Ser Arg Phe Ser Gly
    50 55 60
Ser Gly Ser Gly Thr Glu Phe Thr Leu Thr Ile Ser Ser Leu Gln Pro
65 70 75 80
Asp Asp Phe Ala Ala Tyr Tyr Cys Gln Gln Tyr Asn Asp Tyr Ser Tyr
                  85 90 95
Thr Phe Gly Gln Gly Thr Lys Leu Glu Ile Lys
                100 105
H4H12161P
HCVR
(SEQ ID NO: 50)
Glu Val Gln Leu Val Glu Ser Gly Gly Asp Leu Val Gln Pro Gly Gly
1 5 10 15
Ser Leu Arg Leu Ser Cys Ala Ala Ser Gly Phe Thr Phe Ser Asp His
              20 25 30
Tyr Met Asp Trp Val Arg Gln Ala Pro Gly Lys Gly Leu Asp Trp Ile
            35 40 45
Gly Arg Ile Arg Asn Lys Ala Asn Ala Tyr Asn Thr Glu Tyr Ala Ala
    50 55 60
Ser Val Arg Gly Arg Phe Thr Ile Ser Arg Asp Asp Ser Gln Asn Leu
65 70 75 80
Leu Tyr Leu Gln Met Asn Ser Leu Lys Thr Asp Asp Thr Ala Val Tyr
                  85 90 95
Tyr Cys Val Arg Val Trp Asn Tyr Ala Tyr Phe Ala Met Asp Val Trp
                100 105 110
Gly Gln Gly Thr Thr Val Thr Val Ser Ser
          115 120
LCVR
(SEQ ID NO: 58)
Asp Ile Gln Met Thr Gln Ser Pro Ser Ser Leu Ser Ala Ser Val Gly
1 5 10 15
Asp Arg Val Thr Ile Thr Cys Arg Ser Ser Gln Asn Ile Gly Ile Phe
              20 25 30
Leu Asn Trp Tyr Gln Gln Lys Pro Gly Glu Ala Pro Asn Leu Leu Ile
            35 40 45
Ser Ala Ala Ser Ser Leu His Ser Gly Val Pro Ser Arg Phe Ser Gly
    50 55 60
Ser Gly Ser Gly Thr Asp Phe Thr Leu Thr Ile Gly Ser Leu Gln Pro
65 70 75 80
Glu Asp Phe Ala Thr Tyr Tyr Cys Gln Gln Thr Tyr Asn Thr Ile Phe
                  85 90 95
Thr Phe Gly Pro Gly Thr Lys Val Asp Ile Lys
                100 105
H4H12163P
HCVR
(SEQ ID NO: 66)
Glu Val Gln Leu Val Glu Ser Gly Gly Asp Leu Val Gln Pro Gly Gly
1 5 10 15
Ser Leu Arg Leu Ser Cys Ala Ala Ser Gly Phe Thr Phe Ser Ser Tyr
              20 25 30
Ala Met Asn Trp Val Arg Gln Gly Pro Gly Lys Gly Leu Glu Trp Val
            35 40 45
Ser Ala Ile Ser Gly Arg Gly Asp Ser Thr Tyr Tyr Ala Asp Ser Val
    50 55 60
Lys Gly Arg Leu Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
                  85 90 95
Val Lys Glu Gly Glu Gln Leu Val Tyr Trp Tyr Phe Asp Leu Trp Gly
                100 105 110
Arg Gly Thr Leu Val Thr Val Ser Ser
        115 120
LCVR
(SEQ ID NO: 74)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Thr Ile Ser Asn Phe
              20 25 30
Leu His Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ser Gly
    50 55 60
Ser Gly Ser Gly Thr Asp Phe Thr Leu Thr Ile Ser Ser Leu Gln Pro
65 70 75 80
Glu Asp Phe Ser Thr Tyr Phe Cys Gln Gln Ser Tyr Thr Thr Pro Leu
                  85 90 95
Thr Phe Gly Gly Gly Thr Lys Val Glu Ile Lys
                100 105
H4H12164P
HCVR
(SEQ ID NO: 82)
Glu Val Gln Leu Val Glu Ser Gly Gly Gly Leu Val Arg Ser Gly Gly
1 5 10 15
Ser Leu Arg Leu Ser Cys Ala Ala Ser Gly Phe Thr Phe Asn Arg Tyr
              20 25 30
Ala Met Thr Trp Val Arg Gln Ala Pro Gly Lys Gly Leu Glu Trp Val
            35 40 45
Ser Ala Ile Ser Gly Ser Gly Ser Ser Thr Tyr Tyr Thr Asp Ser Val
    50 55 60
Lys Gly Arg Phe Thr Ile Ser Arg Asp Asn Ser Lys Asn Ser Val Asp
65 70 75 80
Leu Gln Met His Ser Leu Arg Val Glu Asp Thr Ala Ile Tyr Tyr Cys
                  85 90 95
Ala Arg Gly Thr Thr Val Thr Thr Gly Tyr Gly Met Asp Val Trp Gly
                100 105 110
Gln Gly Thr Thr Val Thr Val Ser Ser
        115 120
LCVR
(SEQ ID NO: 90)
Asp Ile Gln Met Thr Gln Ser Pro Ser Ser Leu Ser Ala Ser Val Gly
1 5 10 15
Asp Arg Val Thr Phe Thr Cys Gln Ala Ser Gln Asp Ile Thr Asn Ser
              20 25 30
Leu Asn Trp Tyr Gln Gln Lys Pro Gly Arg Ala Pro Lys Leu Leu Ile
            35 40 45
Tyr Asp Ala Ser Tyr Leu Lys Ala Gly Val Pro Ser Arg Phe Ser Gly
    50 55 60
Ser Gly Ser Gly Thr Asp Phe Thr Phe Thr Ile Ser Ser Leu Gln Pro
65 70 75 80
Glu Asp Ile Ala Thr Tyr Tyr Cys Gln Gln Tyr Asp Asp Leu Pro Tyr
                  85 90 95
Thr Phe Gly Gln Gly Thr Lys Leu Glu Ile Lys
                100 105
H4H12166P
HCVR
(SEQ ID NO: 98)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp Thr Thr Met Ile Phe Asp Tyr Trp Gly Gln
                100 105 110
Gly Thr Leu Val Thr Val Ser Ser
        115 120
LCVR
(SEQ ID NO: 106)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
Thr Phe Gly Gln Gly Thr Lys Val Glu Ile Lys
                100 105
H4H12166P2
HCVR
(SEQ ID NO: 98)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp Thr Thr Met Ile Phe Asp Tyr Trp Gly Gln
                100 105 110
Gly Thr Leu Val Thr Val Ser Ser
        115 120
LCVR
(SEQ ID NO: 114)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys His Gln Asp Phe Asn Tyr Pro Trp
                  85 90 95
Thr Phe Gly Gln Gly Thr Lys Val Glu Ile Lys
                100 105
H4H12166P3
HCVR
(SEQ ID NO: 122)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu His Asn Val Asp Thr Thr Met Ile Phe Asp Tyr Trp Gly Gln
                100 105 110
Gly Thr Leu Val Thr Val Ser Ser
        115 120
LCVR
(SEQ ID NO: 106)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
Thr Phe Gly Gln Gly Thr Lys Val Glu Ile Lys
                100 105
H4H12166P4
HCVR
(SEQ ID NO: 98)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp Thr Thr Met Ile Phe Asp Tyr Trp Gly Gln
                100 105 110
Gly Thr Leu Val Thr Val Ser Ser
        115 120
LCVR
(SEQ ID NO: 130)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
His Phe Gly Gln Gly Thr Lys Val Glu Ile Lys
                100 105
H4H12166P5
HCVR
(SEQ ID NO: 138)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp Thr Thr Met Ile His Asp Tyr Trp Gly Gln
                100 105 110
Gly Thr Leu Val Thr Val Ser Ser
        115 120
LCVR
(SEQ ID NO: 106)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
Thr Phe Gly Gln Gly Thr Lys Val Glu Ile Lys
                100 105
H4H12166P6
HCVR
(SEQ ID NO: 146)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp His Thr Met Ile Phe Asp Tyr Trp Gly Gln
                100 105 110
Gly Thr Leu Val Thr Val Ser Ser
        115 120
LCVR
(SEQ ID NO: 106)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
Thr Phe Gly Gln Gly Thr Lys Val Glu Ile Lys
                100 105
H4H12166P7
HCVR
(SEQ ID NO: 122)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu His Asn Val Asp Thr Thr Met Ile Phe Asp Tyr Trp Gly Gln
                100 105 110
Gly Thr Leu Val Thr Val Ser Ser
        115 120
LCVR
(SEQ ID NO: 130)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
His Phe Gly Gln Gly Thr Lys Val Glu Ile Lys
                100 105
H4H12166P8
HCVR
(SEQ ID NO: 146)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp His Thr Met Ile Phe Asp Tyr Trp Gly Gln
                100 105 110
Gly Thr Leu Val Thr Val Ser Ser
        115 120
LCVR
(SEQ ID NO: 114)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys His Gln Asp Phe Asn Tyr Pro Trp
                  85 90 95
Thr Phe Gly Gln Gly Thr Lys Val Glu Ile Lys
                100 105
H4H12166P9
HCVR
(SEQ ID NO: 146)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp His Thr Met Ile Phe Asp Tyr Trp Gly Gln
                100 105 110
Gly Thr Leu Val Thr Val Ser Ser
        115 120
LCVR
(SEQ ID NO: 130)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
His Phe Gly Gln Gly Thr Lys Val Glu Ile Lys
                100 105
H4H12166P10
HCVR
(SEQ ID NO: 138)
Gln Val Gln Leu Gln Glu Ser Gly Pro Gly Leu Val Lys Pro Ser Glu
1 5 10 15
Thr Leu Ser Leu Thr Cys Thr Val Ser Gly Asp Ser Val Ser Ser Ser
              20 25 30
Tyr Trp Thr Trp Ile Arg Gln Pro Pro Gly Lys Gly Leu Glu Trp Ile
            35 40 45
Gly Tyr Ile Tyr Tyr Ser Gly Ser Ser Asn Tyr Asn Pro Ser Leu Lys
    50 55 60
Ser Arg Ala Thr Ile Ser Val Asp Thr Ser Lys Asn Gln Phe Ser Leu
65 70 75 80
Lys Leu Ser Ser Val Thr Ala Ala Asp Thr Ala Val Tyr Tyr Cys Ala
                  85 90 95
Arg Glu Gly Asn Val Asp Thr Thr Met Ile His Asp Tyr Trp Gly Gln
                100 105 110
Gly Thr Leu Val Thr Val Ser Ser
        115 120
LCVR
(SEQ ID NO: 130)
Ala Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ala Gly
    50 55 60
Arg Gly Ser Gly Thr Asp Phe Thr Leu Thr Ile Ser Ser Leu Gln Pro
65 70 75 80
Glu Asp Phe Ala Thr Tyr Tyr Cys Leu Gln Asp Phe Asn Tyr Pro Trp
                  85 90 95
His Phe Gly Gln Gly Thr Lys Val Glu Ile Lys
                100 105
H4H12167P
HCVR
(SEQ ID NO: 154)
Gln Val Gln Leu Val Glu Ser Gly Gly Gly Leu Val Lys Pro Gly Gly
1 5 10 15
Ser Leu Arg Leu Ser Cys Ala Ala Ser Gly Phe Thr Phe Ser Asp Ser
              20 25 30
Tyr Met Ser Trp Ile Arg Gln Ala Pro Gly Lys Gly Leu Glu Trp Ile
            35 40 45
Ser Tyr Ile Gly Ser Ser Gly Asn Thr Phe Tyr Tyr Ala Asp Ser Val
    50 55 60
Lys Gly Arg Phe Thr Ile Ser Arg Asp Asn Ala Asn Asn Leu Leu Tyr
65 70 75 80
Leu Gln Met Thr Ser Leu Arg Ala Glu Asp Thr Ala Val Tyr Tyr Cys
                  85 90 95
Ala Arg Glu Glu Gly Asp Phe Trp Ser Ala Val Asp Ser Trp Gly Gln
                100 105 110
Gly Thr Leu Val Thr Val Ser Ser
        115 120
LCVR
(SEQ ID NO: 162)
Asp Ile Gln Leu Thr Gln Ser Pro Ser Phe Leu Ser Ala Ser Val Gly
1 5 10 15
Asp Arg Val Thr Ile Thr Cys Trp Ala Ser Gln Gly Ile Ser Ser Tyr
              20 25 30
Leu Ala Trp Tyr Gln Gln Lys Pro Gly Lys Ala Pro Lys Leu Leu Ile
            35 40 45
His Thr Ala Ser Thr Leu Gln Ser Gly Val Pro Ser Arg Phe Ser Gly
    50 55 60
Ser Gly Ser Gly Thr Glu Phe Thr Leu Thr Ile Ser Asn Leu Gln Pro
65 70 75 80
Glu Asp Phe Ala Thr Tyr Tyr Cys Gln Gln Leu Asn Ser Tyr Pro Phe
                  85 90 95
Thr Phe Gly Pro Gly Thr Lys Val Asp Ile Lys
                100 105
H4H12168P
HCVR
(SEQ ID NO: 170)
Gln Val Gln Leu Val Glu Ser Gly Gly Gly Val Val Gln Pro Gly Gly
1 5 10 15
Ser Leu Arg Leu Ser Cys Ala Ala Ser Gly Phe Thr Phe Gly Gly His
              20 25 30
Ala Met His Trp Val Arg Gln Ala Pro Gly Lys Gly Leu Glu Trp Leu
            35 40 45
Ala Val Ile Ser Ser Asp Gly Ser Asn Lys Gln Tyr Ala Asp Ser Val
    50 55 60
Lys Gly Arg Phe Thr Ile Ser Arg Asp Asn Pro Lys Asn Thr Leu Tyr
65 70 75 80
Leu Gln Met Asn Ser Leu Arg Val Gly Asp Thr Ala Ile Tyr Tyr Cys
                  85 90 95
Ala Lys Glu Val Ala Pro Arg Tyr Tyr Tyr Tyr Gly Leu Asp Val Trp
                100 105 110
Gly Gln Gly Thr Thr Val Thr Val Ser Ser
        115 120
LCVR
(SEQ ID NO: 178)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Ser Asn Phe
              20 25 30
Leu Ala Trp Tyr Gln Gln Lys Pro Gly Lys Val Pro Lys Leu Leu Ile
            35 40 45
Tyr Thr Ala Ser Thr Leu Gln Ser Gly Val Pro Ser Arg Phe Ser Gly
    50 55 60
Ser Gly Ser Gly Thr Asp Phe Thr Leu Thr Val Ser Ser Leu Gln Pro
65 70 75 80
Glu Asp Val Ala Thr Tyr Tyr Cys Gln Lys Tyr Ala Gly Ala Leu Thr
                  85 90 95
Phe Gly Pro Gly Thr Lys Val Asp Ile Lys
                100 105
H4H12169P
HCVR
(SEQ ID NO: 186)
Glu Val Gln Leu Val Glu Ser Gly Gly Gly Leu Ala Gln Pro Gly Gly
1 5 10 15
Ser Leu Arg Leu Ser Cys Ala Ala Ser Gly Phe Thr Phe Arg Ser Tyr
              20 25 30
Ala Met Ser Trp Val Arg Gln Ala Pro Gly Lys Gly Pro Glu Trp Val
            35 40 45
Ser Gly Ile Gly Gly Asn Gly Val Thr Thr Tyr Tyr Ala Asp Ser Val
    50 55 60
Lys Gly Arg Phe Thr Ile Ser Arg Asp Asn Ser Lys Asn Thr Leu Phe
65 70 75 80
Leu Gln Met Asn Ser Leu Arg Ala Glu Asp Thr Ala Val Tyr Tyr Cys
                  85 90 95
Val Gln Gly Gly Leu Gly Gly Tyr Phe Thr Gly Tyr Trp Gly Gln Gly
                100 105 110
Thr Leu Val Thr Val Ser Ser
          115
LCVR
(SEQ ID NO: 194)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Ser Ile Ser Thr Tyr
              20 25 30
Leu Asn Trp Tyr Gln Gln Asn Pro Gly Lys Ala Pro Lys Leu Leu Ile
            35 40 45
Phe Asp Ala Ser Ser Leu Gln Ser Gly Val Pro Ser Arg Phe Ser Gly
    50 55 60
Ser Gly Ser Gly Thr Asp Phe Thr Leu Thr Ile Arg Gly Leu Gln Pro
65 70 75 80
Glu Asp Phe Ala Thr Tyr Tyr Cys Gln Gln Ser Tyr Ser Ala Pro Leu
                  85 90 95
Thr Phe Gly Gly Gly Thr Lys Val Glu Ile Lys
                100 105
H4H12170P
HCVR
(SEQ ID NO: 202)
Gln Val Gln Leu Val Glu Ser Gly Gly Gly Val Val Gln Pro Gly Arg
1 5 10 15
Ser Leu Arg Leu Ser Cys Ala Ala Ser Gly Phe Thr Phe Ser Gly Tyr
              20 25 30
Gly Met His Trp Val Arg Gln Ala Pro Gly Lys Gly Leu Glu Trp Val
            35 40 45
Ala Leu Ile Trp Leu Asp Gly Ser Asn Asp Tyr Tyr Ala Asp Ser Val
    50 55 60
Lys Gly Arg Phe Thr Ile Ser Arg Asp Asn Ser Lys Asn Thr Leu Tyr
65 70 75 80
Leu Gln Met Asn Arg Leu Arg Ala Glu Asp Thr Ala Val Tyr Tyr Cys
                  85 90 95
Ala Arg Asp Gly Pro Val Ala Ala Ile Pro Asp Tyr Trp Gly Gln Gly
                100 105 110
Thr Leu Val Thr Val Ser Ser
          115
LCVR
(SEQ ID NO: 210)
Asp Ile Gln Met Thr Gln Ser Pro Ser Thr Leu Ser Ala Ser Val Gly
1 5 10 15
Asp Arg Val Thr Ile Thr Cys Arg Ala Ser Gln Ser Ile Ser Arg Trp
              20 25 30
Leu Ala Trp Tyr Gln Leu Lys Pro Gly Lys Ala Pro Lys Leu Leu Ile
            35 40 45
Tyr Lys Ala Ser Ser Leu Glu Ser Gly Val Pro Ser Arg Phe Ser Gly
    50 55 60
Ser Gly Ser Gly Thr Asp Phe Thr Leu Thr Ile Ser Ser Leu Gln Pro
65 70 75 80
Asp Asp Phe Ala Thr Tyr Tyr Cys Gln Gln Tyr Asn Thr Tyr Ser Tyr
                  85 90 95
Thr Phe Gly Gln Gly Thr Lys Leu Glu Ile Lys
                100 105
H4H12171P
HCVR
(SEQ ID NO: 218)
Glu Val Gln Leu Val Glu Ser Gly Gly Gly Val Val Arg Pro Gly Gly
1 5 10 15
Ser Leu Arg Leu Ser Cys Ala Ala Ser Gly Phe Thr Phe Asp Glu Tyr
              20 25 30
Gly Met Thr Trp Val Arg Gln Val Pro Gly Lys Gly Leu Glu Trp Val
            35 40 45
Ser Gly Ile Thr Trp Asn Gly Gly Phe Thr Asp Tyr Thr Asp Ser Val
    50 55 60
Lys Gly Arg Phe Thr Ser Ser Arg Asp Asn Ala Lys Asn Ser Leu Tyr
65 70 75 80
Leu Gln Met Asn Ser Leu Arg Ala Glu Asp Thr Ala Leu Tyr Tyr Cys
                  85 90 95
Ala Arg Asp Gly Tyr Ser Ser Ser Trp Gly Ala Tyr Asp Ile Trp Gly
                100 105 110
Gln Gly Thr Met Val Thr Val Ser Ser
        115 120
LCVR
(SEQ ID NO: 226)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Ser Ile Ser Thr Tyr
              20 25 30
Leu Asn Trp Tyr Gln Gln Lys Pro Gly Lys Ala Pro Lys Leu Leu Ile
            35 40 45
Tyr Ala Ala Ser Ser Leu Gln Ser Gly Val Pro Leu Arg Phe Ser Gly
    50 55 60
Ser Gly Ser Gly Thr Asp Phe Thr Leu Thr Ile Ser Ser Leu Gln Pro
65 70 75 80
Glu Asp Phe Ala Ser Tyr Phe Cys Gln Gln Ser Tyr Ser Thr Pro Tyr
                  85 90 95
Thr Phe Gly Gln Gly Thr Lys Leu Glu Ile Lys
                100 105
H4H12175P
HCVR
(SEQ ID NO: 234)
Glu Val Gln Leu Val Glu Ser Gly Gly Gly Val Val Gln Pro Gly Gly
1 5 10 15
Ser Leu Arg Leu Ser Cys Ala Ala Ser Gly Phe Thr Phe Asn Asp Tyr
              20 25 30
Ala Met His Trp Val Arg Gln Ala Pro Gly Lys Gly Leu Glu Trp Val
            35 40 45
Ser Leu Ile Ser Gly Asp Gly Gly Asn Thr Tyr Tyr Ala Asp Ser Val
    50 55 60
Lys Gly Arg Leu Thr Ile Ser Arg Asp Asn Ser Lys Asn Ser Leu Tyr
65 70 75 80
Leu Gln Met Asn Ser Leu Arg Thr Glu Asp Thr Ala Leu Tyr Tyr Cys
                  85 90 95
Ala Lys Asp Lys Gly Trp Asn Phe Gly Tyr Phe Asp Leu Trp Gly Arg
                100 105 110
Gly Thr Leu Val Thr Val Ser Ser
        115 120
LCVR
(SEQ ID NO: 242)
Asp Ile Gln Met Thr Gln Ser Pro Ser Ser Leu Ser Thr Ser Val Gly
1 5 10 15
Asp Arg Val Thr Ile Thr Cys Arg Ala Ser Gln Asn Ile Asp Thr Tyr
              20 25 30
Leu Asn Trp Tyr Gln Gln Lys Pro Gly Lys Ala Pro Lys Leu Leu Ile
            35 40 45
Tyr Asp Ala Ser Ser Leu Gln Ser Gly Val Pro Ser Arg Phe Ser Gly
    50 55 60
Ser Gly Ser Gly Thr Asp Phe Thr Leu Thr Ile Thr Ser Leu Gln Pro
65 70 75 80
Glu Asp Phe Ala Thr Tyr Tyr Cys Gln Gln Asn Asp Asn Ile Leu His
                  85 90 95
Pro Leu Thr Phe Gly Gly Gly Thr Lys Val Glu Ile Lys
                100 105
H4H12176P2
HCVR
(SEQ ID NO: 250)
Glu Val Gln Leu Val Glu Ser Gly Gly Gly Leu Val Gln Pro Gly Gly
1 5 10 15
Ser Leu Arg Leu Ser Cys Ala Ala Ser Gly Phe His Ser Asn Arg Tyr
              20 25 30
Trp Met Asp Trp Val Arg Gln Ala Pro Gly Lys Gly Leu Glu Trp Val
            35 40 45
Ala Asn Ile Lys Gln Asp Gly Ser Glu Glu Asn Tyr Val Asp Ser Val
    50 55 60
Lys Gly Arg Phe Thr Ile Ser Arg Asp Asn Ala Lys Asn Ser Leu Tyr
65 70 75 80
Leu Gln Met Asn Ser Leu Arg Ala Glu Asp Thr Ala Val Tyr Tyr Cys
                  85 90 95
Ala Arg Asp Arg Ser Thr Ser Trp Val Pro Tyr Trp Phe Phe Asp Leu
                100 105 110
Trp Gly Arg Gly Thr Leu Val Thr Val Ser Ser
        115 120
LCVR
(SEQ ID NO: 258)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Ser Ile Ser Ser Tyr
              20 25 30
Leu Asn Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ser Gly
    50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Ser Tyr Ser Thr Pro Pro
                  85 90 95
Ile Thr Phe Gly Gln Gly Thr Arg Leu Glu Ile Lys
                100 105
H4H12177P2
HCVR
(SEQ ID NO: 266)
Glu Val Gln Leu Val Glu Ser Gly Gly Gly Val Val Gln Arg Gly Glu
1 5 10 15
Ser Leu Arg Leu Ser Cys Ser Ala Ser Asp Phe Ile Phe Lys Asp Tyr
              20 25 30
Ala Met Tyr Trp Val Arg Gln Ile Pro Gly Lys Gly Leu Glu Trp Ile
            35 40 45
Ser Leu Ile Ser Gly Asp Gly Asp Thr Thr Trp Tyr Gly Asp Ser Val
    50 55 60
Lys Gly Arg Phe Thr Ile Ser Arg Asp Asn Asn Glu Asn Ser Leu Phe
65 70 75 80
Leu Gln Met Asn Asp Leu Arg Thr Glu Asp Thr Ala Met Tyr Tyr Cys
                  85 90 95
Ala Arg Asp Met Gly Trp Asn Phe Phe Gln Leu Gln Tyr Trp Gly Gln
                100 105 110
Gly Thr Leu Val Thr Val Ser Ser
        115 120
LCVR
(SEQ ID NO: 258)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Ser Ile Ser Ser Tyr
              20 25 30
Leu Asn Trp Tyr Gln Gln Lys Pro Gly Lys Ala Pro Lys Leu Leu Ile
            35 40 45
Tyr Ala Ala Ser Ser Leu Gln Ser Gly Val Pro Ser Arg Phe Ser Gly
    50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Ser Tyr Ser Thr Pro Pro
                  85 90 95
Ile Thr Phe Gly Gln Gly Thr Arg Leu Glu Ile Lys
                100 105
H4H12183P2
HCVR
(SEQ ID NO: 274)
Gln Val Gln Leu Gln Glu Ser Gly Pro Ala Leu Val Lys Pro Ser Gln
1 5 10 15
Thr Leu Ser Leu Thr Cys Thr Val Ser Gly Gly Ser Ile Ile Arg Gly
              20 25 30
Ser Thr Tyr Trp Ser Trp Val Arg Gln Phe Pro Gly Lys Gly Leu Glu
            35 40 45
Trp Ile Gly Tyr Ser Tyr Tyr Ser Gly Thr Ala Tyr Tyr Asn Pro Ser
    50 55 60
Leu Glu Ser Arg Ala Thr Ile Ser Val Asp Thr Ser Lys Asn Gln Phe
65 70 75 80
Ser Leu Asn Leu Lys Ser Val Thr Ala Ala Asp Thr Ala Val Tyr Tyr
                  85 90 95
Cys Thr Arg Glu Ile Gly Val Ala Gly Leu Phe Asp Ile Trp Gly Gln
                100 105 110
Gly Thr Leu Val Thr Val Ser Ser
        115 120
LCVR
(SEQ ID NO: 282)
Glu Ile Val Leu Thr Gln Ser Pro Gly Thr Leu Ser Leu Ser Pro Gly
1 5 10 15
Glu Arg Ala Thr Leu Ser Cys Arg Ala Ser Gln Ser Val Ser Ser Ser
              20 25 30
Tyr Leu Ala Trp Tyr Gln Gln Lys Pro Gly Gln Ala Pro Arg Leu Leu
            35 40 45
Ile Tyr Gly Ala Ser Ser Arg Ala Thr Gly Ile Pro Asp Arg Phe Ser
    50 55 60
Gly Ser Gly Ser Gly Thr Asp Phe Thr Leu Thr Ile Ser Arg Leu Glu
65 70 75 80
Pro Glu Asp Phe Ala Val Tyr Tyr Cys Gln Gln Tyr Gly Ser Ser Pro
                  85 90 95
Trp Thr Phe Gly Gln Gly Thr Lys Val Glu Ile Lys
                100 105
H2M11682N
HCVR
(SEQ ID NO: 290)
Gln Glu Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Gly Tyr
              20 25 30
Tyr Ile His Trp Val Arg Gln Ala Pro Gly Leu Gly Leu Glu Trp Met
            35 40 45
Gly Trp Ile Asn Pro Asn Ser Gly Gly Thr Lys Tyr Ala Gln Lys Phe
    50 55 60
Gln Gly Arg Val Thr Met Thr Arg Asp Thr Ser Ile Asn Thr Ala Tyr
65 70 75 80
Met Glu Leu Lys Arg Leu Lys Ser Asp Asp Ser Ala Val Tyr Tyr Cys
                  85 90 95
Ala Arg Asp Ala Pro Pro His Asp Val Phe Asp Ile Trp Gly Gln Gly
                100 105 110
Thr Leu Val Thr Val Ser Ser
          115
LCVR
(SEQ ID NO: 298)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Asp
              20 25 30
Leu Gly Trp Tyr Gln Gln Lys Pro Gly Lys Ala Pro Lys Arg Leu Ile
            35 40 45
Tyr Ala Ala Ser Ser Leu Gln Ile Gly Val Pro Ser Arg Phe Ser Gly
    50 55 60
Ser Gly Ser Gly Thr Glu Phe Thr Leu Thr Ile Ser Ser Leu Gln Pro
65 70 75 80
Glu Asp Phe Ala Thr Tyr Tyr Cys Leu Gln His Asn Ser Tyr Pro Leu
                  85 90 95
Thr Phe Gly Gly Gly Thr Lys Val Glu Ile Lys
                100 105
H2M11684N
HCVR
(SEQ ID NO: 306)
Gln Val Gln Leu Gln Glu Ser Gly Pro Gly Leu Val Lys Pro Ser Gln
1 5 10 15
Thr Leu Ser Leu Thr Cys Thr Val Ser Gly Gly Ser Ile Ser Ser Gly
              20 25 30
Ala Tyr His Trp Ser Trp Ile Arg Gln His Pro Gly Lys Gly Leu Glu
            35 40 45
Trp Ile Gly Tyr Ile Tyr Tyr Asn Gly Asp Thr Tyr Tyr Asn Pro Ser
    50 55 60
Leu Lys Ser Arg Val Thr Ile Ser Val Asp Thr Ser Lys Asn Gln Phe
65 70 75 80
Phe Leu Lys Val Thr Ser Val Thr Ala Ala Asp Thr Ala Met Tyr Tyr
                  85 90 95
Cys Ala Gly Glu Lys Gln Leu Thr Ala Phe Asp Ile Trp Gly Gln Gly
                100 105 110
Thr Leu Val Thr Val Ser Ser
          115
LCVR
(SEQ ID NO: 314)
Val Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Asn Asn Phe
              20 25 30
Leu Asn Trp Tyr Gln Gln Lys Leu Gly Lys Ala Pro Lys Leu Leu Ile
            35 40 45
Ser Asp Ala Ser Asn Leu Gln Thr Gly Val Pro Ser Arg Phe Ser Gly
    50 55 60
Ser Gly Ser Gly Thr Asp Phe Thr Phe Thr Ile Ser Ser Leu Gln Pro
65 70 75 80
Glu Asp Ile Ala Ala Tyr Tyr Cys Gln Gln Tyr Asp His Phe Pro Tyr
                  85 90 95
Thr Phe Gly Gln Gly Thr Arg Leu Glu Asn Asn
                100 105
H2M11694N
HCVR
(SEQ ID NO: 322)
Glu Val Gln Leu Val Glu Ser Gly Gly Gly Val Val Arg Pro Gly Gly
1 5 10 15
Ser Leu Arg Leu Ser Cys Ala Ala Ser Gly Phe Thr Phe Asp Asp Tyr
              20 25 30
Gly Met Thr Trp Val Arg Gln Ala Pro Gly Lys Gly Leu Glu Trp Val
            35 40 45
Ser Gly Ile Asn Trp Asn Gly Asp Ser Thr Glu Tyr Ser Asp Ser Val
    50 55 60
Lys Gly Arg Phe Thr Ile Ser Arg Asp Asn Ala Lys Asn Ser Leu Tyr
65 70 75 80
Leu Gln Met Asn Ser Leu Arg Ala Glu Asp Thr Ala Phe Tyr His Cys
                  85 90 95
Ala Arg Glu Asn Asn Trp Asn Phe Tyr Phe Asp Tyr Trp Gly Gln Gly
                100 105 110
Thr Leu Val Thr Val Ser Ser
          115
LCVR
(SEQ ID NO: 330)
Glu Ile Val Met Thr Gln Ser Pro Ala Thr Leu Ser Val Ser Arg Gly
1 5 10 15
Glu Arg Ala Thr Leu Ser Cys Arg Ala Ser Gln Ser Val Ser Ser Asn
              20 25 30
Leu Ala Trp Tyr Gln Gln Lys Leu Gly Gln Ala Pro Arg Leu Leu Ile
            35 40 45
Tyr Gly Ala Ser Thr Arg Ala Thr Gly Ile Pro Ala Arg Phe Ser Gly
    50 55 60
Ser Gly Ser Gly Thr Glu Phe Thr Leu Thr Ile Ser Ser Leu Gln Ser
65 70 75 80
Glu Asp Phe Ala Val Tyr Tyr Cys Gln Gln Tyr Asn Asn Trp Pro Trp
                  85 90 95
Thr Phe Gly Gln Gly Thr Lys Val Glu Ile Lys
                100 105
H2M11695N
HCVR
(SEQ ID NO: 338)
Gln Val His Leu Val Gln Ser Gly Ala Glu Val Lys Lys Pro Gly Ala
1 5 10 15
Ser Val Lys Val Ser Cys Lys Val Ser Gly Asn Thr Leu Thr Glu Leu
              20 25 30
Ser Met His Trp Val Arg Gln Ala Pro Gly Lys Gly Leu Glu Trp Met
            35 40 45
Gly Gly Phe Asp Pro Glu Asp Gly Asp Thr Ile Tyr Ser Gln Lys Phe
    50 55 60
Gln Gly Arg Val Thr Leu Thr Glu Asp Thr Ser Thr Asp Thr Ala Tyr
65 70 75 80
Met Glu Leu Ser Ser Leu Arg Ser Glu Asp Thr Ala Val Tyr Tyr Cys
                  85 90 95
Ser Thr Val Gly Gly Pro Thr Ser Asp Cys Trp Gly Gln Gly Thr Leu
                100 105 110
Val Thr Val Ser Ser
          115
LCVR
(SEQ ID NO: 346)
Asp Ile Gln Met Thr Gln Ser Pro Ser Ser Leu Ser Ala Ser Val Gly
1 5 10 15
Asp Arg Val Thr Ile Thr Cys Gln Ala Ser Gln Asp Ile Ser Asn Tyr
              20 25 30
Leu Asn Trp Tyr Gln Gln Lys Pro Gly Lys Ala Pro Lys Val Leu Ile
            35 40 45
Phe Asp Ala Ser Asn Leu Glu Pro Gly Val Pro Ser Arg Phe Ser Gly
    50 55 60
Ser Gly Ser Gly Thr Asp Phe Thr Phe Thr Ile Ile Ser Leu Gln Pro
65 70 75 80
Glu Asp Ile Ala Thr Tyr Tyr Cys Gln Gln Tyr Asp Asn Leu Pro Ile
                  85 90 95
Thr Phe Gly Gln Gly Thr Arg Leu Asp Ile Lys
                100 105
```

In an embodiment of the invention, any antigen-binding protein that binds specifically to C5 (anti-C5) which is discussed herein is an antagonist. Such an antagonist (e.g., an antagonist antigen-binding protein that binds specifically to C5) binds to C5 and inhibits at least one biological activity of C5; for example, preventing or blocking complement-mediated hemolysis by classical pathway or alternative pathway and/or inhibits cleavage of C5 to C5a and C5b and/or inhibits complement mediated lysis of red blood cells and/or inhibits formation of membrane attack complex (MAC) and/or inhibits formation of C5b-6 complex.

In an embodiment of the invention, the antagonist antigen-binding protein that binds specifically to C5 is eculizumab (sold as Soliris), ravulizumab (ALXN1210; sold as Ultomiris), tesidolumab (see U.S. Pat. No. 8,241,628; WO 2010/015608; or WO2017/212375) or mubodina (see U.S. Pat. No. 7,999,081); or an antigen-binding fragment thereof. In an embodiment of the invention, the antagonist antigen-binding protein that binds specifically to C5 is pozelimab (REGN3918; H4H12166P) antibody; or an antigen-binding fragment thereof. Pozelimab (REGN3918; H4H12166P) antibody comprises a heavy chain immunoglobulin comprising the amino acid sequence:

(SEQ ID NO: 368)
QVQLQESGPG LVKPSETLSL TCTVSGDSVS SSYWTWIRQP PGKGLEWIGY IYYSGSSNYN 60
PSLKSRATIS VDTSKNQFSL KLSSVTAADT AVYYCAREGN VDTTMIFDYW GQGTLVTVSS 120
ASTKGPSVFP LAPCSRSTSE STAALGCLVK DYFPEPVTVS WNSGALTSGV HTFPAVLQSS 180
GLYSLSSVVT VPSSSLGTKT YTCNVDHKPS NTKVDKRVES KYGPPCPPCP APEFLGGPSV 240
FLFPPKPKDT LMISRTPEVT CVVVDVSQED PEVQFNWYVD GVEVHNAKTK PREEQFNSTY 300
RVVSVLTVLH QDWLNGKEYK CKVSNKGLPS SIEKTISKAK GQPREPQVYT LPPSQEEMTK 360
NQVSLTCLVK GFYPSDIAVE WESNGQPENN YKTTPPVLDS DGSFFLYSRL TVDKSRWQEG 420
NVFSCSVMHE ALHNHYTQKS LSLSLGK; 447

and a light chain immunoglobulin comprising the amino acid sequence:

(SEQ ID NO: 369)
AIQMTQSPSS LSASVGDRVT ITCRASQGIR NDLGWYQQKP GKAPKLLIYA ASSLQSGVPS 60
RFAGRGSGTD FTLTISSLQP EDFATYYCLQ DFNYPWTFGQ GTKVEIKRTV AAPSVFIFPP 120
SDEQLKSGTA SVVCLLNNFY PREAKVQWKV DNALQSGNSQ ESVTEQDSKD STYSLSSTLT 180
LSKADYEKHK VYACEVTHQG LSSPVTKSFN RGEC. 214

The present invention includes methods for using antagonist antigen-binding proteins that bind specifically to C5, e.g., antibodies and antigen-binding fragments thereof, that include the variable regions (V_{H} and V_{L}) and/or CDRs (V_{L} with LCDR1, LCDR2 and LCDR3; and V_{H} with HCDR1, HCDR2 and HCDR3) which are specifically discussed herein (e.g., of Pozelimab) as well as variable regions and CDRs which are variants of those discussed herein.

A “variant” of a polypeptide, such as an immunoglobulin chain (e.g., the H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab, eculizumab, tesidolumab or mubodina V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2; 4; 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68; 70; 72; 74; 76; 78; 80; 82; 84; 86; 88; 90; 92; 94; 96; 98; 100; 102; 104; 106; 108; 110; 112; 114; 116; 118; 120; 122; 124; 126; 128; 130; 132; 134; 136; 138; 140; 142; 144; 146; 148; 150; 152; 154; 156; 158; 160; 162; 164; 166; 168; 170; 172; 174; 176; 178; 180; 182; 184; 186; 188; 190; 192; 194; 196; 198; 200; 202; 204; 206; 208; 210; 212; 214; 216; 218; 220; 222; 224; 226; 228; 230; 232; 234; 236; 238; 240; 242; 244; 246; 248; 250; 252; 254; 256; 258; 260; 262; 264; 266; 268; 270; 272; 274; 276; 278; 280; 282; 284; 286; 288; 290; 292; 294; 296; 298; 300; 302; 304; 306; 308; 310; 312; 314; 316; 318; 320; 322; 324; 326; 328; 330; 332; 334; 336; 338; 340; 342; 344; 346; 348; 350 and/or 352); see e.g., Table A; when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain specifically set forth herein (e.g., the H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab, eculizumab, tesidolumab or mubodina V_{H}, V_{L}, HC or LC or CDR thereof); but including one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present invention includes methods for using antagonist antigen-binding proteins that bind specifically to C5, e.g., antibodies and antigen-binding fragments thereof, which include an immunoglobulin light chain (or V_{L}) variant comprising the amino acid sequence set forth in SEQ ID NO: 106 but having one or more of such mutations and/or an immunoglobulin heavy chain (or V_{H}) variant comprising the amino acid sequence set forth in SEQ ID NO: 98 but having one or more of such mutations. In an embodiment of the invention, the antagonist antigen-binding protein that binds specifically to C5 includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y. “H2M11683N”; “H2M11686N”; “H4H12159P”; “H4H12161P”; “H4H12163P”;

“H4H12164P”; “H4H12166P”; “H4H12166P2”; “H4H12166P3”; “H4H12166P4”; “H4H12166P5”; “H4H12166P6”; “H4H12166P7”; “H4H12166P8”; “H4H12166P9”; “H4H12166P10”; “H4H12167P”; “H4H12168P”; “H4H12169P”; “H4H12170P”; “H4H12171P”; “H4H12175P”; “H4H12176P2”; “H4H12177P2”; “H4H12183P2”; “H2M11682N”; “H2M11684N”; “H2M11694N” or “H2M11695N”, unless otherwise stated, refer to antagonist antigen-binding proteins that bind specifically to C5, e.g., antibodies and antigen-binding fragments thereof (including multi-specific antigen-binding proteins), that bind specifically to C5 (e.g., human C5), comprising an immunoglobulin heavy chain or variable region thereof (V_{H}) comprising the amino acid sequence specifically set forth herein corresponding, in Table A, to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; or H2M11695N (e.g., SEQ ID NO: 2; 18; 34; 50; 66; 82; 98; 138; 146; 122; 146; 154; 170; 186; 202; 218; 234; 250; 266; 274; 290; 306; 322 or 338) (or a variant thereof), and/or an immunoglobulin light chain or variable region thereof (V_{L}) comprising the amino acid sequence specifically set forth herein corresponding, in Table A, to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 10; 26; 42; 58; 74; 90; 106; 114; 130; 162; 178; 194; 210; 226; 242; 258; 282; 298; 314; 330 or 346) (or a variant thereof), respectively; and/or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof))—or, see International Patent Application Publicatoin No. WO2017/218515. In an embodiment of the invention, the V_{H} is linked to a constant heavy chain domain, such as a human constant heavy chain domain (e.g., IgG, IgG1 or IgG4 (e.g., IgG4 (S228P mutant, Eu numbering))) and/or the V_{L} is linked to a constant light chain domain, such as a human constant light chain domain (e.g., lambda or kappa). In an embodiment of the invention, the heavy chain constant domain is IgG4 having he S108P mutation.

In an embodiment of the invention, the antagonist antigen-binding protein that binds specifically to C5, H2M11683N, comprises an HCVR that comprises the amino acid sequence set forth in SEQ ID NO: 2 and an LCVR that comprises the amino acid sequence set forth in SEQ ID NO: 10 (e.g., wherein the antigen-binding protein is an antibody or antigen-binding fragment thereof).

In an embodiment of the invention, the antagonist antigen-binding protein that binds specifically to C5, H2M11686N, comprises an HCVR that comprises the amino acid sequence set forth in SEQ ID NO: 18 and an LCVR that comprises the amino acid sequence set forth in SEQ ID NO: 26 (e.g., wherein the antigen-binding protein is an antibody or antigen-binding fragment thereof).

In an embodiment of the invention, the antagonist antigen-binding protein that binds specifically to C5, H4H12159P, comprises an HCVR that comprises the amino acid sequence set forth in SEQ ID NO: 34 and an LCVR that comprises the amino acid sequence set forth in SEQ ID NO: 42 (e.g., wherein the antigen-binding protein is an antibody or antigen-binding fragment thereof).

In an embodiment of the invention, the antagonist antigen-binding protein that binds specifically to C5, H4H12161P, comprises an HCVR that comprises the amino acid sequence set forth in SEQ ID NO: 50 and an LCVR that comprises the amino acid sequence set forth in SEQ ID NO: 58 (e.g., wherein the antigen-binding protein is an antibody or antigen-binding fragment thereof).

In an embodiment of the invention, the antagonist antigen-binding protein that binds specifically to C5, H4H12163P, comprises an HCVR that comprises the amino acid sequence set forth in SEQ ID NO: 66 and an LCVR that comprises the amino acid sequence set forth in SEQ ID NO: 74 (e.g., wherein the antigen-binding protein is an antibody or antigen-binding fragment thereof).

In an embodiment of the invention, the antagonist antigen-binding protein that binds specifically to C5, H4H12164P, comprises an HCVR that comprises the amino acid sequence set forth in SEQ ID NO: 82 and an LCVR that comprises the amino acid sequence set forth in SEQ ID NO: 90 (e.g., wherein the antigen-binding protein is an antibody or antigen-binding fragment thereof).

In an embodiment of the invention, the antagonist antigen-binding protein that binds specifically to C5, H4H12166P, comprises an HCVR that comprises the amino acid sequence set forth in SEQ ID NO: 98 and an LCVR that comprises the amino acid sequence set forth in SEQ ID NO: 106 (e.g., wherein the antigen-binding protein is an antibody or antigen-binding fragment thereof).

In an embodiment of the invention, the antagonist antigen-binding protein that binds specifically to C5, H4H12166P2, comprises an HCVR that comprises the amino acid sequence set forth in SEQ ID NO: 98 and an LCVR that comprises the amino acid sequence set forth in SEQ ID NO: 114 (e.g., wherein the antigen-binding protein is an antibody or antigen-binding fragment thereof).

In an embodiment of the invention, the antagonist antigen-binding protein that binds specifically to C5, H4H12166P3, comprises an HCVR that comprises the amino acid sequence set forth in SEQ ID NO: 122 and an LCVR that comprises the amino acid sequence set forth in SEQ ID NO: 106 (e.g., wherein the antigen-binding protein is an antibody or antigen-binding fragment thereof).

In an embodiment of the invention, the antagonist antigen-binding protein that binds specifically to C5, H4H12166P4, comprises an HCVR that comprises the amino acid sequence set forth in SEQ ID NO: 98 and an LCVR that comprises the amino acid sequence set forth in SEQ ID NO: 130 (e.g., wherein the antigen-binding protein is an antibody or antigen-binding fragment thereof).

In an embodiment of the invention, the antagonist antigen-binding protein that binds specifically to C5, H4H12166P5, comprises an HCVR that comprises the amino acid sequence set forth in SEQ ID NO: 138 and an LCVR that comprises the amino acid sequence set forth in SEQ ID NO: 106 (e.g., wherein the antigen-binding protein is an antibody or antigen-binding fragment thereof).

In an embodiment of the invention, the antagonist antigen-binding protein that binds specifically to C5, H4H12166P6, comprises an HCVR that comprises the amino acid sequence set forth in SEQ ID NO: 146 and an LCVR that comprises the amino acid sequence set forth in SEQ ID NO: 106 (e.g., wherein the antigen-binding protein is an antibody or antigen-binding fragment thereof).

In an embodiment of the invention, the antagonist antigen-binding protein that binds specifically to C5, H4H12166P7, comprises an HCVR that comprises the amino acid sequence set forth in SEQ ID NO: 122 and an LCVR that comprises the amino acid sequence set forth in SEQ ID NO: 130 (e.g., wherein the antigen-binding protein is an antibody or antigen-binding fragment thereof).

In an embodiment of the invention, the antagonist antigen-binding protein that binds specifically to C5, H4H12166P8, comprises an HCVR that comprises the amino acid sequence set forth in SEQ ID NO: 146 and an LCVR that comprises the amino acid sequence set forth in SEQ ID NO: 114 (e.g., wherein the antigen-binding protein is an antibody or antigen-binding fragment thereof).

In an embodiment of the invention, the antagonist antigen-binding protein that binds specifically to C5, H4H12166P9, comprises an HCVR that comprises the amino acid sequence set forth in SEQ ID NO: 146 and an LCVR that comprises the amino acid sequence set forth in SEQ ID NO: 130 (e.g., wherein the antigen-binding protein is an antibody or antigen-binding fragment thereof).

In an embodiment of the invention, the antagonist antigen-binding protein that binds specifically to C5, H4H12166P10, comprises an HCVR that comprises the amino acid sequence set forth in SEQ ID NO: 138 and an LCVR that comprises the amino acid sequence set forth in SEQ ID NO: 130 (e.g., wherein the antigen-binding protein is an antibody or antigen-binding fragment thereof).

In an embodiment of the invention, the antagonist antigen-binding protein that binds specifically to C5, H4H12167P, comprises an HCVR that comprises the amino acid sequence set forth in SEQ ID NO: 154 and an LCVR that comprises the amino acid sequence set forth in SEQ ID NO: 162 (e.g., wherein the antigen-binding protein is an antibody or antigen-binding fragment thereof).

In an embodiment of the invention, the antagonist antigen-binding protein that binds specifically to C5, H4H12168P, comprises an HCVR that comprises the amino acid sequence set forth in SEQ ID NO: 170 and an LCVR that comprises the amino acid sequence set forth in SEQ ID NO: 178 (e.g., wherein the antigen-binding protein is an antibody or antigen-binding fragment thereof).

In an embodiment of the invention, the antagonist antigen-binding protein that binds specifically to C5, H4H12169P, comprises an HCVR that comprises the amino acid sequence set forth in SEQ ID NO: 186 and an LCVR that comprises the amino acid sequence set forth in SEQ ID NO: 194 (e.g., wherein the antigen-binding protein is an antibody or antigen-binding fragment thereof).

In an embodiment of the invention, the antagonist antigen-binding protein that binds specifically to C5, H4H12170P, comprises an HCVR that comprises the amino acid sequence set forth in SEQ ID NO: 202 and an LCVR that comprises the amino acid sequence set forth in SEQ ID NO: 210 (e.g., wherein the antigen-binding protein is an antibody or antigen-binding fragment thereof).

In an embodiment of the invention, the antagonist antigen-binding protein that binds specifically to C5, H4H12171P, comprises an HCVR that comprises the amino acid sequence set forth in SEQ ID NO: 218 and an LCVR that comprises the amino acid sequence set forth in SEQ ID NO: 226 (e.g., wherein the antigen-binding protein is an antibody or antigen-binding fragment thereof).

In an embodiment of the invention, the antagonist antigen-binding protein that binds specifically to C5, H4H12175P, comprises an HCVR that comprises the amino acid sequence set forth in SEQ ID NO: 234 and an LCVR that comprises the amino acid sequence set forth in SEQ ID NO: 242 (e.g., wherein the antigen-binding protein is an antibody or antigen-binding fragment thereof).

In an embodiment of the invention, the antagonist antigen-binding protein that binds specifically to C5, H4H12176P2, comprises an HCVR that comprises the amino acid sequence set forth in SEQ ID NO: 250 and an LCVR that comprises the amino acid sequence set forth in SEQ ID NO: 258 (e.g., wherein the antigen-binding protein is an antibody or antigen-binding fragment thereof).

In an embodiment of the invention, the antagonist antigen-binding protein that binds specifically to C5, H4H12177P2, comprises an HCVR that comprises the amino acid sequence set forth in SEQ ID NO: 266 and an LCVR that comprises the amino acid sequence set forth in SEQ ID NO: 258 (e.g., wherein the antigen-binding protein is an antibody or antigen-binding fragment thereof).

In an embodiment of the invention, the antagonist antigen-binding protein that binds specifically to C5, H4H12183P2, comprises an HCVR that comprises the amino acid sequence set forth in SEQ ID NO: 274 and an LCVR that comprises the amino acid sequence set forth in SEQ ID NO: 282 (e.g., wherein the antigen-binding protein is an antibody or antigen-binding fragment thereof).

In an embodiment of the invention, the antagonist antigen-binding protein that binds specifically to C5, H2M11682N, comprises an HCVR that comprises the amino acid sequence set forth in SEQ ID NO: 290 and an LCVR that comprises the amino acid sequence set forth in SEQ ID NO: 298 (e.g., wherein the antigen-binding protein is an antibody or antigen-binding fragment thereof).

In an embodiment of the invention, the antagonist antigen-binding protein that binds specifically to C5, H2M11684N, comprises an HCVR that comprises the amino acid sequence set forth in SEQ ID NO: 306 and an LCVR that comprises the amino acid sequence set forth in SEQ ID NO: 314 (e.g., wherein the antigen-binding protein is an antibody or antigen-binding fragment thereof).

In an embodiment of the invention, the antagonist antigen-binding protein that binds specifically to C5, H2M11694N, comprises an HCVR that comprises the amino acid sequence set forth in SEQ ID NO: 322 and an LCVR that comprises the amino acid sequence set forth in SEQ ID NO: 330 (e.g., wherein the antigen-binding protein is an antibody or antigen-binding fragment thereof).

In an embodiment of the invention, the antagonist antigen-binding protein that binds specifically to C5, H2M11695N, comprises an HCVR that comprises the amino acid sequence set forth in SEQ ID NO: 338 and an LCVR that comprises the amino acid sequence set forth in SEQ ID NO: 346 (e.g., wherein the antigen-binding protein is an antibody or antigen-binding fragment thereof).

Thus, the present invention includes antigen-binding proteins comprising the V_{H} and V_{L} variable domains set forth herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab, eculizumab, tesidolumab or mubodina) which are linked to a heavy and/or light chain constant domain, respectively, e.g., as set forth above (e.g., a V_{H} linked to a human IgG4 heavy chain constant region and a V_{L} linked to a human kappa light chain constant region).

The term “antibody”, as used herein, refers to immunoglobulin molecules comprising four polypeptide chains, two heavy chains (HCs) including three H-CDRs and two light chains (LCs) including three L-CDRs, inter-connected by disulfide bonds (e.g. IgG4)-for example H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; or H2M11695N. In an embodiment of the invention, the assignment of amino acids to each CDR domain within an immunoglobulin chain is in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883. Thus, the present invention includes antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined according to Kabat and/or Chothia.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that does not include all sequences of an antibody, but which specifically binds an antigen (e.g., C5). Non-limiting examples of antigen-binding fragments include: (i) F(ab) and F(ab′) fragments; (ii) F(ab′)₂ fragments; (iii) Fd fragments (heavy chain portion of a Fab fragment cleaved with papain); (iv) Fv fragments (a V_{H} or V_{L}); and (v) single-chain Fv (scFv) molecules; having the amino acid residues that mimic the hypervariable region of an antibody (e.g., an isolated complementarity determining region (CDR) such as a CDR3 peptide), or a constrained FR3-CDR3-FR4 peptide. Other engineered molecules, such as single domain antibodies, domain-deleted antibodies, minibodies and small modular immunopharmaceuticals (SMIPs), are also encompassed within the expression “antigen-binding fragment,” as used herein. In an embodiment of the invention, the antigen-binding fragment comprises three or more CDRs of H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; or H2M11695N (e.g., CDR-H1, CDR-H2 and CDR-H3; and/or CDR-L1, CDR-L2 and CDR-L3).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present invention includes methods for using recombinant antigen-binding proteins as set forth herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; or H2M11695N).

The present invention includes methods for using monoclonal antagonist antigen-binding proteins that bind specifically to C5 (e.g., antibodies and antigen-binding fragments thereof). The term “monoclonal antibody” or “mAb”, as used herein, refers to an antibody from a population of substantially homogeneous antibodies, i.e., the antibody molecules comprising the population are identical in amino acid sequence except for possible naturally occurring mutations that may be present in minor amounts. The modifier “monoclonal” is not to be construed as requiring production of the antibody by any particular method. Monoclonal antibodies may be made by the hybridoma method of Kohler et al. (1975) Nature 256: 495, or may be made by recombinant DNA methods (see, e.g., U.S. Pat. No. 4,816,567).

“Isolated” antagonist antigen-binding proteins that bind specifically to C5 (e.g., antibodies or antigen-binding fragments thereof), polypeptides, polynucleotides and vectors, are at least partially free of other biological molecules from the system, cells or cell culture from which they are produced. Such biological molecules include nucleic acids, proteins, other antibodies or antigen-binding fragments, lipids, carbohydrates, or other material such as cellular debris and growth medium. An isolated antigen-binding protein may be at least partially free of the growth medium in which a host cell expressing the antigen-binding protein is grown. Generally, the term “isolated” is not intended to be limited to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

An “anti-C5” antigen-binding protein specifically binds to C5 (e.g., human C5 or cynomolgous monkey C5). The term “specifically binds” refers to those antigen-binding proteins (e.g., mAbs) having a binding affinity to an antigen at 25° C., expressed as K_{D}, of at least about 10⁻⁹ M or less (a lower number) (e.g., about 10⁻¹⁰M, about 10⁻¹¹ M or about 10⁻¹² M), as measured by real-time, label free bio-layer interferometry assay, e.g., an Octet® HTX biosensor, or by surface plasmon resonance, e.g., BIACORE™, or by solution-affinity ELISA. In an embodiment of the invention, the K_{D} for binding to human C5 is about 189 pM; for binding to human C5 (R885C or R885H) is about 400-500 pM; and for binding to cynomolgus monkey C5 is about 2-3 nM at 25° C., pH7.4, by surface plasmon resonance assay. In an embodiment of the invention, human C5 (including the signal sequence) comprises the amino acid sequence set forth in SEQ ID NO: 362; and mature human C5 comprising the mutation R885H comprises the amino acid sequence set forth in SEQ ID NO: 363.

### Dosing and Administration

The present invention includes methods for the treatment or prevention of a C5-associated disease and/or for ameliorating at least one sign or symptom associated with such C5-associated disease, in a subject, by administering an antagonist antigen-binding protein that binds specifically to C5 (e.g., REGN3918) to the subject as follows: (i) administering one or more doses (e.g., 1 dose) of about 30 mg/kg (body weight (BW)) of the antigen-binding protein intravenously (IV); then (ii) administering either one or more doses (e.g., 2 or more) of about 800 mg of the antigen-binding protein (e.g., subcutaneously (SC)) (this may be referred to, herein, as the 30+800 dosing regimen), or one or more SC doses according to body weight as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; and for BW ≥60 kg: about 800 mg. Such SC dose(s) may be given on a weekly basis following the initial IV dose(s). The weekly doses can be continued indefinitely, for example, as long as a therapeutic effect or prevention of an undesired outcome (e.g., loss of serum albumin, or increase in serum LDH levels) is desired. Optionally, the subject is administered one or more doses of an oligonucleotide (e.g., cemdisiran) in association with the antigen-binding protein.

In an embodiment of the invention, the antagonist antigen-binding protein that binds specifically to C5 (e.g., pozelimab) is administered to a patient in a method as set forth herein (e.g., for treating or preventing PNH or CHAPLE) with the proviso that no other agent which reduces complement activity (e.g., that reduces C5 activity), for example, an oligonucleotide (e.g., that reduces C5 expression) such as cemdisiran, or an antibody or antigen-binding fragment thereof that binds specifically to C5, is administered to the patient.

The present invention also includes methods for treating or preventing a C5-associated disease (e.g., PNH or CHAPLE) by administering one or more doses (e.g., one or more than one) of about 30 mg/kg (body weight (BW)) of the antagonist antigen-binding protein that binds specifically to C5 (e.g., REGN3918) or pharmaceutical formulation thereof, intravenously (IV). An intravenous dose of 30 mg/kg has been demonstrated to help to quickly achieve the steady-state trough concentrations of the antigen-binding protein (e.g., antibody) required for sustained maximal CH50 inhibition which, thus, would lead to a therapeutic effect in the subject. Optional, further subcutaneous doses of antigen-binding protein may be given to the subject, e.g., weekly, e.g., following the IV dose(s).

In an embodiment of the invention, the subject (e.g., who suffers from PNH) is administered: (i) about 30 mg/kg of antagonist antigen-binding protein that binds specifically to C5 intravenously (IV) initially (day 1); then (ii) about 800 mg of the antigen-binding protein (e.g., subcutaneously (SC)) once a week (e.g., ±1, ±2 or +3 days), e.g., on about day 8 (e.g., ±1, ±2 or +3 days), 15 (e.g., ±1, ±2 or +3 days), 22 (e.g., ±1, ±2 or +3 days), etc., and every week (e.g., ±1, ±2 or ±3 days) thereafter.

The present invention includes a method for treating or preventing CHAPLE disease in a subject comprising administering, to the subject, a therapeutically effective dose of antagonist antigen-binding protein that binds specifically to C5 selected from H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; and H2M11695N, or pharmaceutical formulation thereof (e.g., 30 mg/kg intravenous). In an embodiment of the invention, the subject (e.g., who suffers from CHAPLE) is administered:

(i) about 30 mg/kg of the antigen-binding protein intravenously (IV) (on day 1); then
(ii) starting at about day 8 (e.g., day 8, day 8±1 day, day 8±2 days or day 8±3 days), one or more doses administered subcutaneously (SC), and continuing thereafter on a weekly basis, at doses depending on body weight (BW) as follows:
For body weight (BW)<10 kg: about 125 mg;
For BW ≥10 kg and <20 kg: about 200 mg;
For BW ≥20 kg and <40 kg: about 350 mg;
For BW ≥40 kg and <60 kg: about 500 mg; and
For BW ≥60 kg: about 800 mg.

Dosing once a week or weekly dosing or QW dosing refers to administering one or more doses where each occurs about 7 (e.g., ±1, ±2 or ±3) days after the immediately preceding dose.

In an embodiment of the invention, the IV and first SC dose are given on the same day.

In an embodiment of the invention, the antagonist antigen-binding protein that binds specifically to C5, when administered subcutaneously (SC), is delivered in less than 7 ml volume, about 0.625 ml, about 1 ml, about 1.75 ml, about 2.5 ml, about 4 ml, about 0.5-4.0 ml, or about 0.625-4.0 ml. In an embodiment of the invention, each SC dose is delivered in a single injection. In an embodiment of the invention, the SC injection is delivered in about 60 seconds or less.

In an embodiment of the invention, a subject (e.g., who suffers from a C5-associated disease) is administered one or more doses of an antagonist antigen-binding protein that binds specifically to C5 as follows: 1 mg/kg IV; 3 mg/kg IV; 300 mg SC; 800 mg SC; 10 mg/kg IV; 600 mg SC; or 30 mg/kg IV; or, a loading dose of 15 mg/kg IV followed by one or more SC doses of 400 mg administered once weekly.

A serum concentration of about 100 mg/liter antagonist antigen-binding protein that binds specifically to C5 (e.g., REGN3918) in a human subject maximally suppresses C5 activity (e.g., alternative, classical and lectin pathways) (e.g., as measured by AH50 and/or CH50 assay). Thus, the present invention includes methods for suppressing complement activity or C5 activity (e.g., alternative pathway (AP)) (e.g., suppressing C5 activity to about its maximal level (e.g., at least about 80%, 81%, 82%, 83%, 84%, 85%, 86%, 87%, 88%, 89%, 90%, 91%, 92%, 93%, 94%, 95%, 96%, 97%, 98%, 99% or 100%); e.g., which is measured as AH50 and/or CH50 activity) in a subject comprising administering one or more doses of the antigen-binding protein at sufficient levels so as to maintain the serum concentration of the anti-C5 antigen-binding protein at about 100 mg/liter or more (e.g., 150, 400, 600 or 700 mg/liter). In an embodiment of the invention, the dosing regimen comprises

(i) administering one or more doses (e.g., 1 dose) of about 30 mg/kg (body weight (BW)) of the antigen-binding protein intravenously (IV); then, optionally,
(ii) administering one or more weekly doses (e.g., 2 or more) of about 800 mg of the antigen-binding protein (e.g., subcutaneously (SC));
or,
one or more weekly doses, administered subcutaneously (SC) depending on body weight (BW), as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; or for BW ≥60 kg: about 800 mg. The weekly doses can be continued indefinitely, for example, as long as maintenance of the serum concentration of the anti-C5 antigen-binding protein and/or suppression of the C5 activity is desired.

The present invention includes methods for achieving or achieving and maintaining a serum concentration (e.g., a steady state serum trough concentration over time) of about 100 mg/liter or more antagonist antigen-binding protein that binds specifically to C5 in a subject comprising: (i) administering one or more doses (e.g., 1 dose) of about 30 mg/kg (body weight (BW)) of the antigen-binding protein intravenously (IV); then, optionally, (ii) administering one or more weekly doses (e.g., 2 or more) of about 800 mg of the antigen-binding protein (e.g., subcutaneously (SC)); or one or more weekly doses, administered subcutaneously (SC) depending on body weight (BW) as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; or for BW ≥60 kg: about 800 mg. The weekly doses can be continued indefinitely, for example, as long as maintenance of the anti-C5 antigen-binding protein serum concentration is desired.

The present invention further provides methods for switching a subject from a therapeutic regimen that includes administration of eculizumab or ravulizumab to a therapeutic regimen that includes administration of an antagonist antigen-binding protein that binds specifically to C5 selected from: H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; and H2M11695N; comprising administering an initial dose of the antigen-binding protein to the subject when the next dose is due in the eculizumab or ravulizumab therapeutic regimen and ceasing further administrations of the eculizumab or ravulizumab. In an embodiment of the invention, the initial dose of the antigen-binding protein is about 30 mg/kg (body weight (BW)) of the antigen-binding protein intravenously (IV), which is optionally followed by one or more further IV doses. In an embodiment of the invention, following the IV dose(s), the subject is administered one or more weekly subcutaneous doses (e.g., 2 or more) of about 800 mg of the antigen-binding protein; or one or more weekly subcutaneous doses depending on body weight (BW) as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; or for BW ≥60 kg: about 800 mg. In an embodiment of the invention, such a switching method excludes overlapping the eculizumab or ravulizumab dosing regimens with the dosing regimen of the antagonist antigen-binding protein that binds specifically to C5.

In an embodiment of the invention, the intravenous infusion of the antagonist antigen-binding protein that binds specifically to C5 is interrupted and restarted at 50% of the original infusion rate if, during the infusion, the subject suffers from one or more adverse events, such as, for example: cough, rigors/chills, Rash, pruritus (itching), urticaria (hives, welts, wheals), diaphoresis (sweating), hypotension, dyspnea (shortness of breath), vomiting or flushing.

The term “C5-associated disease” refers to a disease, disorder, condition or syndrome which is caused, maintained or exacerbated, or whose signs and/or symptoms are caused, maintained or exacerbated, directly or indirectly, by complement system activity wherein the complement system activity can be reduced or stabilized or eliminated by inhibition of C5 activity. Such C5 activity can be inhibited by preventing, for example, cleavage of C5 precursor into C5a and C5b chains, formation of membrane attack complex (MAC) and/or binding of the MAC to the surface of a target cell (e.g., a red blood cell). In an embodiment of the invention, C5 activity inhibition is as measured in a CH50 assay.

CH50 (50% Hemolytic Complement) is an assay to determine the level of classical complement pathway and it is sensitive to the reduction, absence and/or inactivity of any component of the pathway which is well known in the art. CH50 tests the functional capability of serum complement components of the classical pathway to lyse, for example, sheep red blood cells (SRBC) pre-coated with rabbit anti-sheep red blood cell antibody (haemolysin). For example, when antibody-coated SRBC are incubated with test serum, the classical pathway of complement is activated and hemolysis results. If a complement component is absent, the CH50 level will be zero; if one or more components of the classical pathway are decreased, the CH50 will be decreased. A fixed volume of optimally sensitized SRBC is added to each serum dilution. For example, after incubation, the mixture is centrifuged and the degree of hemolysis is quantified by measuring the absorbance of the hemoglobin released into the supernatant at 540 nm. The amount of complement activity is determined by examining the capacity of various dilutions of test serum to lyse antibody coated SRBC. See Costabile, Measuring the 50% haemolytic complement (CH50) activity of serum, J Vis Exp. 2010 (37): 1923; and Mayer, Complement and complement fixation, 1 p. 133-240. In E. A. 2 Kabat and M. M. Mayer (ed.), Experimental immunochemistry. Thomas, Springfield. AH50 is an analogous test to measure alternate-pathway function. See e.g., Mayer, Complement and complement fixation, p. 133-240. In E. Kabat and M. M. Mayer (ed.), Experimental immunochemistry. C. C. Thomas, Springfield, Ill. 1961; and Rapp & Borsos. Molecular basis of complement action. Appton Century Crofts, New York, N.Y. 1970. Tests evaluating the functional activity of the alternative pathway (AH50) use guinea pig, rabbit, or chicken erythrocytes as target cells. The AP has weak hemolytic activity for sheep erythrocytes. Here, activation of the classical pathway has to be blocked by adding EGTA to chelate 2′, and an optimal concentration of Mg²⁺ is required. Detection of low or absent hemolytic activity in CH50 and/or AH50 directs further complement analysis. See e.g., Joiner et al., 1983. A study of optimal reaction conditions for an assay of the human alternative complement pathway. Am. J. Clin. Pathol. 79:65-72.

A therapeutically effective amount of antagonist antigen-binding protein that binds specifically to C5 is an amount that reverses, stabilizes or eliminates an undesired disease or disorder (e.g., a C5-associated disease), for example, by causing the regression, stabilization or elimination of one or more signs or symptoms of such disease or disorder by any clinically measurable degree, e.g., with regard to C5-associated disease, by causing a reduction in or maintenance of complement activity. The dosage regimens set forth herein are examples of therapeutically effective amounts of the antagonist antigen-binding proteins.

The term “treat” or “treatment” refers to a therapeutic measure that reverses, stabilizes or eliminates an undesired disease or disorder (e.g., a C5-associated disease such as PNH, MG, aHUS or CHAPLE), for example, by causing the regression, stabilization or elimination of one or more signs or symptoms of such disease or disorder by any clinically measurable degree, e.g., with regard to C5-associated disease, by causing a reduction in or maintenance of complement activity.

Subjective evidence of a disease, disorder, condition or syndrome is a symptom. A sign is objective evidence of the disease, disorder, condition or syndrome. For example, blood coming out a nostril is a sign insofar as it is apparent to the patient, physician, and others. Anxiety, low back pain, and fatigue are symptoms insofar as only the patient can perceive them.

The term “subject” refers to a mammal such as a human, mouse, goat, rabbit, rat, dog, non-human primate or monkey. In an embodiment of the invention, amino acid Arginine 885 is mutated in the subject's C5 (e.g. human C5) to another amino acid, e.g., R885H or R885C. In an embodiment of the invention, a subject has previously received an antagonist antigen-binding protein that binds specifically to C5 other than what is currently being administered, e.g., wherein the subject previously received ravulizumab or eculizumab.

A C5-associated disease is, for example:
adult respiratory distress syndrome
age-related macular degeneration (AMD)
allergy
Alport's syndrome
Alzheimer's disease
Amyotrophic lateral sclerosis (ALS)
antiphospholipid syndrome (APS)
asthma
atherosclerosis
atypical hemolytic uremic syndrome (aHUS)
an autoimmune disease
autoimmune hemolytic anemia (AIHA)
balloon angioplasty
bronchoconstriction
bullous pemphigoid
burns
C3 glomerulopathy
capillary leak syndrome
a cardiovascular disorder
catastrophic antiphospholipid syndrome (CAPS)
a cerebrovascular disorder
CHAPLE disease (CD55 deficiency with hyperactivation of complement, angiopathic thrombosis, and protein-losing enteropathy)
a chemical injury
chronic obstructive pulmonary disease (COPD)
cold agglutinin disease (CAD)
corneal and/or retinal tissue
Crohn's disease
Degos disease
dense deposit disease (DDD)
dermatomyositis
diabetes
diabetic angiopathy
diabetic macular edema (DME)
diabetic nephropathy
diabetic retinopathy
dilated cardiomyopathy
disorder of inappropriate or undesirable complement activation
dyspnea
eclampsia
emphysema
epidermolysis bullosa
epilepsy
fibrogenic dust disease
frostbite
geographic atrophy (GA)
glomerulonephritis
glomerulopathy
Goodpasture's Syndrome
Graves' disease
Guillain-Barre Syndrome
Hashimoto's thyroiditis
hemodialysis complications
hemolysis-elevated liver enzymes- and low platelets (HELLP) syndrome
hemolytic anemia
hemoptysis
Henoch-Schonlein purpura nephritis
hereditary angioedema
hyperacute allograft rejection
hypersensitivity pneumonitis
idiopathic thrombocytopenic purpura (ITP)
IgA nephropathy
an immune complex disorder
immune complex vasculitis
immune complex-associated inflammation
an infectious disease
inflammation caused by an autoimmune disease
an inflammatory disorder
inherited CD59 deficiency
injury due to inert dusts and/or minerals
interleukin-2 induced toxicity during IL-2 therapy
ischemia-reperfusion injury
Kawasaki's disease
a lung disease or disorder
lupus nephritis
membrane proliferative glomerulonephritis
membranoproliferative nephritis
mesenteric artery reperfusion after aortic reconstruction
mesenteric/enteric vascular disorder
multifocal motor neuropathy (MMN)
multiple sclerosis
myasthenia gravis
myocardial infarction
myocarditis
neurological disorder
neuromyelitis optica
obesity
ocular angiogenesis
ocular neovascularization affecting choroidal
organic dust disease
parasitic disease
Parkinson's disease
paroxysmal nocturnal hemoglobinuria (PNH), e.g., active PNH
pauci-immune vasculitis
pemphigus
percutaneous transluminal coronary angioplasty (PTCA)
peripheral (e.g., musculoskeletal) vascular disorder
pneumonia
post-ischemic reperfusion condition
post-pump syndrome in cardiopulmonary bypass
post-pump syndrome in renal bypass
pre-eclampsia
progressive kidney failure
proliferative nephritis
proteinuric kidney disease
psoriasis
pulmonary embolism
pulmonary fibrosis
pulmonary infarction
pulmonary vasculitis
recurrent fetal loss
a renal disorder
renal ischemia
renal ischemia-reperfusion injury
a renovascular disorder
restenosis following stent placement
rheumatoid arthritis (RA)
rotational atherectomy
schizophrenia
sepsis
septic shock
SLE nephritis
smoke injury
spinal cord injury
spontaneous fetal loss
stroke
systemic inflammatory response to sepsis
systemic lupus erythematosus (SLE)
systemic lupus erythematosus-associated vasculitis
Takayasu's disease
thermal injury
thrombotic thrombocytopenic purpura (TTP)
traumatic brain injury
type I diabetes
typical hemolytic uremic syndrome (tHUS)
uveitis
vasculitis
vasculitis associated with rheumatoid arthritis
venous gas embolus (VGE); or
xenograft rejection.

Thus, the present invention includes methods for treating or preventing a C5-associated disease (e.g., PNH or aHUS), in a subject (e.g., a human) in need thereof e.g., in a subject suffering from the C5-associated disease, comprising administering an antagonist antigen-binding protein that binds specifically to C5 (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab or eculizumab) to the subject according to a dosing regimen set forth herein, optionally in association with a further therapeutic agent (e.g., cemdisiran). In addition, the present invention provides methods for reducing the need for therapeutic interventions needed to address various signs and symptoms of C5-associated diseases such as PNH or CHAPLE.

Paroxysmal nocturnal hemoglobinuria (PNH) originates from a multipotent, hematopoietic stem cell (HSC) that acquires a mutation of the phosphatidylinositol glycan anchor biosynthesis class A (PIGA) gene. The PIGA gene product is required for the biosynthesis of the glycophosphatidylinositol (GPI) anchor, a glycolipid moiety that attaches dozens of proteins to the plasma membrane of cells. Consequently, the PNH stem cell and all of its progeny have a reduction or absence of GPI-anchored proteins. The mature blood cells derived from the hematopoietic clone can have a complete deficiency (type III) or a partial deficiency (type II) of GPI-linked proteins (Hillmen et al., Effect of eculizumab on hemolysis and transfusion requirements in patients with paroxysmal nocturnal hemoglobinuria. N Engl J Med 2004; 350(6):552-9). Two of the proteins that are affected by the absence of GPI anchors are CD55 and CD59, complement regulatory proteins. CD55 regulates complement activation by inhibiting complement component 3 (C3) convertases, whereas CD59 inhibits the assembly of the membrane-attack complex (MAC) C5b-C9 by interacting with C8 and C9 (Brodsky, How I treat paroxysmal nocturnal hemoglobinuria. Blood 2009; 113(26):6522-7). Their absence renders PNH erythrocytes susceptible to complement-mediated intravascular hemolysis. This intravascular hemolysis in patients with PNH causes anemia (frequently requiring blood transfusion) and hemoglobinuria. Complications of PNH include thrombosis, abdominal pain, dysphagia, erectile dysfunction, and pulmonary hypertension (Hillmen et al., The complement inhibitor eculizumab in paroxysmal nocturnal hemoglobinuria. N Engl J Med 2006; 355(12):1233-43). Thromboembolism is a common cause of mortality in patients with PNH. Potential mechanisms for thromboembolism include platelet activation, toxicity of free hemoglobin, nitric oxide depletion, absence of other GPI-linked proteins, and endothelial dysfunction (Hill et al., Thrombosis in paroxysmal nocturnal hemoglobinuria. Blood 2013; 121(25):4985-96). PNH frequently occurs with autoimmune aplastic anemia (Luzzatto & Risitano, Advances in understanding the pathogenesis of acquired aplastic anaemia. Br J Haematol 2018; 182(6):758-76). The present invention includes methods for reducing the need for blood transfusions to address anemia secondary to hemolysis that is caused by PNH, reducing the need for erythropoietin, iron supplements and/or folic acid, reducing the incidence of anemia, reducing the incidence of hemoglobinuria or reducing the incidence of hemolysis, in a subject suffering from PNH, by administering, to the subject, an antagonist antigen-binding protein that binds specifically to C5 such as REGN3918 by a dosing regimen set forth herein.

The diagnosis of PNH can be established using an internationally accepted definition of presence of PNH granulocyte clone size of >10% measured in peripheral blood by flow cytometry. An accepted definition of “active disease” (active PNH) is the presence of 1 or more of the following PNH-related signs or symptoms within 3 months: fatigue, hemoglobinuria, abdominal pain, shortness of breath (dyspnea), anemia (hemoglobin <10 g/dL), history of a major adverse vascular event (MAVE; including thrombosis), dysphagia, or erectile dysfunction. Alternatively, activity can be established by a history of RBC transfusion due to PNH within 3 months. Methods for treating active PNH are also included within the scope of the present invention.

CHAPLE disease (CD55 deficiency with hyperactivation of complement, angiopathic thrombosis, and protein-losing enteropathy) is an autosomal recessive disorder caused by loss of function mutations in CD55 (also known as decay accelerating factor, DAF). Signs and symptoms of CHAPLE can include hypoproteinemia (low serum levels of albumin and immunoglobulins)-hypoproteinemia leads to facial and extremity edema and recurrent infections, malabsorption syndrome (chronic diarrhea, failure to thrive, anemia, and micronutrient deficiencies), complement overactivation, intestinal lymphangiectasia (IL) and bowel inflammation; and/or increased susceptibility to visceral thrombosis. CHAPLE disease is caused by biallelic loss-of-function mutations in the CD55 gene. Clinically, it manifests as a familial form of protein-losing enteropathy (PLE) caused by primary intestinal lymphangiectasia (PIL) or Waldmann's disease that is frequently severe and can be accompanied by lethal systemic manifestations. CD55 is a glycophosphatidylinositol (GPI)-anchored membrane protein that inhibits the enzymatic activity of C3b and C4b, thus preventing the formation of C3 and C5 convertases that lead ultimately to the assembly of the membrane-attack complex (C5b-C9). Thus, the absence of CD55 causes overactivation of the complement system, causing the production of various complement products including anaphylatoxins and the membrane-attack complex. When absent due to somatic mutation of the PIGA gene (required for the biosynthesis of GPI anchors) in hematopoietic stem cells, CD55 loss, as well as CD59 loss, is specific to hematopoietic cells (CD59 is another GPI-linked complement regulatory protein). Typically, the resultant complement-mediated lysis of red cells and platelets gives rise to intravascular hemolysis and thrombosis in PNH. In CHAPLE, isolated germ line loss of CD55 expression in all tissues manifests in the GI tract, as primary intestinal lymphangiectasia, which causes PLE. In general, unlike PNH, hemolysis is not observed in CHAPLE patients. The present invention includes methods for reducing the need for the administration of corticosteroids, immunoglobulin, albumin, biological therapeutic agents (e.g., antibodies or antigen-binding fragments thereof such as anti-TNFalpha, or vedolizumab), immunomodulators (e.g., azathioprine or mesalazine), micronutrients, enteral or parenteral supplementation, anti-coagulants (e.g., low-molecular-weight heparin), antibiotics and/or anti-platelet agents (e.g., aspirin, such as low-dose aspirin), in a subject suffering from CHAPLE, by administering, to the subject, an antagonist antigen-binding protein that binds specifically to C5, such as REGN3918, by a dosing regimen set forth herein. See Kurolap et al., Loss of CD55 in Eculizumab-Responsive Protein-Losing Enteropathy. N Engl J Med 2017; 377(1):87-9.; and Ozen et al., CD55 Deficiency and Protein-Losing Enteropathy. N Engl J Med 2017b; 377(15):1499-500.

CD55 mutations associated with CHAPLE disease include, for example,

149-150delAA;
149-150insCCTT;
109delC;

### 800G>C;

### 287-1G>C;

149-150delAAinsCCTT;
(as set forth in WO2018/053039)
or a CD55 mutation that results in the same mutant amino acid sequence. Thus, the present invention includes methods for treating CHAPLE disease characterized by any one or more of such mutations. In an embodiment of the invention, human CD55 comprises the amino acid sequence set forth in SEQ ID NO: 364; human CD55 comprising the mutation Glu50Alafs*12 comprises the amino acid sequence set forth in SEQ ID NO: 365 (residues 101-200); human CD55 comprising the mutation Gly37Alafs*24 comprises the amino acid sequence set forth in SEQ ID NO: 366 (residues 1-100); and human CD55 comprising the mutation Cys267Ser comprises the amino acid sequence set forth in SEQ ID NO: 367 (residues 201-300) (see International patent application publication no. WO2018/053039).

Diagnosis of CHAPLE can be done by genetic analysis to identify a CD55 loss-of-function mutation. Diagnosis can be confirmed by flow cytometry or Western blotting of peripheral blood cells to identify decreased presence of CD55. Active CHAPLE disease is, in an embodiment of the invention, characterized by hypoalbuminemia of less than or equal to 3.2 g/dL, and one or more of the following signs or symptoms which are attributable to CHAPLE: diarrhea, vomiting, abdominal pain, peripheral or facial edema, or an episode of infection with concomitant hypogammaglobulinemia, or a new thromboembolic event. The normal range of serum albumin is typically about 3.5-5.5 g/dL.

Atypical hemolytic uremic syndrome (aHUS) is a rare disease characterized by low levels of circulating red blood cells due to their destruction (hemolytic anemia), low platelet count (thrombocytopenia) due to their consumption and inability of the kidneys to process waste products from the blood and excrete them into the urine (acute kidney failure), a condition known as uremia. Most aHUS are caused by complement system defects impairing ordinary regulatory mechanisms. Activating events therefore lead to unbridled, ongoing complement activity producing widespread endothelial injury. Signs and symptoms of aHUS can include, for example, feelings of illness, fatigue, irritability, and lethargy, anemia, thrombocytopenia, acute kidney failure, hypertension and organ damage.

Antiphospholipid syndrome (APS) is an autoimmune disease characterized by arterial and venous thrombosis due to antiphospholipid antibodies. The disorder is referred to as primary when it occurs in the absence of another autoimmune disease. Secondary APS occurs in the context of an autoimmune disorder such as systemic lupus erythematosus. The catastrophic APS (CAPS) is a rare life-threatening form of APS in which widespread intravascular thrombosis results in multiorgan ischemia and failure.

Myasthenia gravis (MG) is a chronic autoimmune neuromuscular disease that causes weakness in the skeletal muscles, which are responsible for breathing and moving parts of the body, including the arms and legs.

Typical hemolytic uremic syndrome (tHUS) may follow a gastrointestinal infection with Shiga toxin-producing Escherichia coli (STEC). Typical HUS (STEC-HUS; Shiga toxin-producing Escherichia coli (STEC)-hemolytic uremic syndrome (HUS)) can be initiated when the Shiga toxin (or Shiga-like toxin), a known potent cytotoxin, binds to cell membrane glycolipid Gb3 (via domain B). Domain A is internalized and subsequently halts protein synthesis and induces apoptosis of the affected cell. The Shiga toxin has several additional effects on endothelial cells, one of which is enhanced expression of functional tissue factor that could contribute to microvascular thrombosis. The toxin causes damage to or activation of endothelium, red cells, and platelets.

The present invention provides methods for administering an antagonist antigen-binding protein that binds specifically to C5 (e.g., REGN3918) to a subject comprising (i) administering one or more doses (e.g., 1 dose) of about 30 mg/kg (body weight (BW)) of the antigen-binding protein (e.g., REGN3918) intravenously (IV); then, optionally, (ii) administering one or more doses (e.g., 2 or more) of about 800 mg of the antigen-binding protein (e.g., subcutaneously (SC)). Such SC dose(s) may be given on a weekly basis following the initial IV dose(s). The present invention also provides methods for administering an antigen-binding protein (e.g., REGN3918) to a subject comprising (i) administering one or more doses (e.g., 1 dose) of about 30 mg/kg (body weight (BW)) of the antigen-binding protein intravenously (IV); then, optionally, (ii) administering one or more SC doses according to body weight as follows: for body weight (BW)<10 kg: about 125 mg; for BW >10 kg and <20 kg: about 200 mg; for BW ≥20 kg and <40 kg: about 350 mg; for BW ≥40 kg and <60 kg: about 500 mg; and for BW ≥60 kg: about 800 mg. Such SC dose(s) may be given on a weekly basis following the initial IV dose(s). Optionally, the subject is administered one or more doses of an oligonucleotide (e.g., cemdisiran) in association with the antigen-binding protein. In an embodiment of the invention, the subject suffers from a C5-associated disease such as, for example, CHAPLE, PNH, aHUS or MG.

### Diagnostics

The present invention includes methods for treating or preventing a C5-associated disease such as PNH. PNH can be diagnosed in a subject, for example, on the basis of:

(i) Flow cytometry analysis of peripheral blood;
(ii) Serum lactate dehydrogenase (LDH) level ≥2×upper limit of normal (ULN); and/or
(iii) PNH granulocytes (denoted as polymorphonuclear [PMN]) >10%.

Flow cytometry analysis of peripheral blood is a means for laboratory detection of PNH. Flow cytometric immunophenotyping is performed to detect the presence or absence of GPI-linked proteins on granulocytes, monocytes, and erythrocytes, using fluorescently labeled monoclonal antibodies or FLAER (Fluorescein-Labeled Proaerolysin). FLAER is a fluorescently labeled variant of aerolysin that binds directly to the GPI anchor and can be used to evaluate the expression of the GPI linkage. Individuals with PNH have decreased or absent expression of CD14 on monocytes, CD16 on neutrophils and NK cells, CD24 on neutrophils, CD59 on red blood cells and FLAER on neutrophils and monocytes.

Proaerolysin is a 52-kDa protein secreted by Aeromonas hydrophila. After proteolytic nicking at the C-terminus, the active form, Aerolysin, is generated that binds to cell surface structures and oligomerizes, forming channels that result in cell lysis (Howard & Buckley, Activation of the hole-forming toxin aerolysin by extracellular processing. J. Bacteriol. 1985; 163:336-340). Aerolysin does not lyse PNH cells and it was shown that the toxin bound to the GPI moiety of GPI-linked structures (Diep et al., Glycosyl-phosphatidylinositol anchors of membrane glycoproteins are binding determinants for the channel-forming toxin aerolysin. J. Biol. Chem. 1998; 273:2355-2360.25.; Brodsky et al., Resistance of paroxysmal nocturnal hemoglobinuria cells to the glycosylphosphatidylinositol-binding toxin aerolysin. Blood 1999; 93:1749-1756). Initially, this reagent was used to enrich rare GPI-negative PNH clones. Subsequently, a fluorochrome-conjugated (Alexa 488) version of a non-lysing, mutated form of proaerolysin (FLAER) was generated that retained specificity for GPI-linked structures without causing cell lysis.

PNH is characterized by chronic uncontrolled terminal complement activation and hemolysis. Uncontrolled complement activation leads to red blood cell (RBC) hemolysis, platelet activation and subsequently thromboembolism (TE), renal and other organ impairment, pain, severe fatigue, poor quality of life and early mortality. An indicator of cell lysis is the appearance of abnormally high levels of lactate dehydrogenase (LDH) in the serum. An LDH serum level of ≥1.5 or 2.0× the upper limit of normal (LDH ≥1.5×; LDH ≥2.0×) is a marker of uncontrolled complement activation that has been used in multinational PNH clinical trials. Normal serum levels of LDH can vary depending on the laboratory and methods used for measurements; however, in children, the normal level is about 60-170 U/L and in adults it is about 100-190 U/L. Other reports have the normal adult LDH range as 140-280 U/L. In an embodiment of the invention, the normal female LDH ULN is 330 U/L and the male LDH ULN is 281 U/L. Having received one or more red blood cell transfusions, e.g., within 3 months, is also an indicator of PNH.

A large population GPI-AP (glycosyl phosphatidylinositol anchored protein)—deficient PMNs (polymorphonuclear cells) is also an indicator of PNH. Flow cytometry is a means by which to determine the presence of such PMNs.

Signs and symptoms of PNH also include fatigue, hemoglobinuria, abdominal pain, shortness of breath (dyspnea), anemia (hemoglobin <10 g/dL), a history of a major adverse vascular events (MAVE, including thrombosis), dysphagia, or erectile dysfunction.

CHAPLE disease, for example, can be diagnosed on the basis of a genotype characterized by a biallelic CD55 loss-of-function mutation and persistent protein-losing enteropathy (PLE). In an embodiment of the invention, active CHAPLE disease can be identified in a patient exhibiting: hypoalbuminemia of less than or equal to 3.2 g/dL; and within the last 6 months and attributable to CD55-deficient PLE, at least 7 days (which do not have to be consecutive) of at least one of the following symptoms or signs: diarrhea, vomiting, abdominal pain, peripheral or facial edema, or an episode of infection with concomitant hypogammaglobulinemia, or a new thromboembolic event. Other characteristics upon which a diagnosis of CHAPLE disease may be based include, e.g., primary intestinal lymphangiectasia or Waldmann's disease, growth retardation, anemia, vitamin or micronutrient deficiency, GI mucosal ulcer, Lymphoid infiltrates in GI mucosa, recurrent lung infection, hypothyroidism, arthritis, arthralgia or finger clubbing. See e.g., Ozen et al., CD55 Deficiency, Early-Onset Protein-Losing Enteropathy, and Thrombosis, New England J. of Med. 377(1): 52-61 (2017).

The present invention includes methods for treating or preventing a C5-associated disease (e.g., PNH), in a subject, by

(i) evaluating the subject for the presence of signs and/or symptoms of the disease, and, diagnosing the subject with the disease if one or more of such signs and/or symptoms are identified (e.g., as discussed herein);

and

(ii) administering an antagonist antigen-binding protein that binds specifically to C5 (e.g., antibody or antigen-binding fragment thereof; e.g., REGN3918) to the subject according to a dosing regimen of the present invention—e.g., (i) administering one or more doses (e.g., 1 dose) of about 30 mg/kg (body weight (BW)) of the antigen-binding protein intravenously (IV); then, optionally, (ii) administering one or more doses (e.g., 2 or more) of about 800 mg of the antigen-binding protein subcutaneously (SC). In an embodiment of the invention, SC doses are given on a weekly basis. In an embodiment of the invention, said signs and symptoms include LDH level ≥1.5 or 2×ULN; Type III PNH granulocytes >10%; and/or signs and symptoms of active PNH disease.

The present invention includes methods for treating or preventing a C5-associated disease (e.g., CHAPLE), in a subject, by

(i) evaluating the subject for the presence of signs and/or symptoms of the disease, e.g., CHAPLE, and, diagnosing the subject with the disease if one or more of such signs and/or symptoms are identified (e.g., as discussed herein);

and

(ii) administering one or more doses (e.g., 1 dose) of about 30 mg/kg (body weight (BW)) of antagonist antigen-binding protein that binds specifically to C5 intravenously (IV); then (ii) administering one or more SC doses according to body weight as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; and for BW ≥60 kg: about 800 mg. In an embodiment of the invention, SC doses are given on a weekly basis. In an embodiment of the invention, such signs and symptoms include loss-of-function mutation in the CD55 gene, flow cytometry or Western blot of peripheral blood cells to identify reduced presence of CD55, hypoalbuminemia of less than or equal to 3.2 g/dL and/or one or more of: diarrhea, vomiting, abdominal pain, peripheral or facial edema, or an episode of infection with concomitant hypogammaglobulinemia, or a new thromboembolic event.

### Pharmaceutical Formulations and Compositions

The present invention provides methods for treating or preventing a C5-associated disease comprising administering an antagonist antigen-binding protein that binds specifically to C5 (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab, eculizumab, tesidolumab or mubodina) according to a dosing regimen of the present invention (e.g., (i) administering one or more doses of about 30 mg/kg (body weight (BW)) of the antigen-binding protein intravenously (IV); then, optionally, (ii) administering either one or more weekly SC doses of about 800 mg of the antigen-binding protein; or one or more weekly SC doses according to body weight as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; and for BW ≥60 kg: about 800 mg; optionally, in association with one or more further therapeutic agents (e.g., an oligonucleotide such as cemdisiran). In an embodiment of the invention, an antagonist antigen-binding protein that binds specifically to C5 administered to a subject is in a pharmaceutical formulation that includes a pharmaceutically acceptable carrier. A pharmaceutically acceptable carrier includes one or more excipients. In an embodiment of the invention, a pharmaceutical formulation of the present invention is aqueous, i.e., includes water. In an embodiment of the invention, the pharmaceutical formulation comprises about 200 mg/ml antagonist antigen-binding protein that binds specifically to C5.

Pharmaceutical formulations including antagonist antigen-binding protein that binds specifically to C5 (e.g., REGN3918) may be prepared by admixing the antigen-binding protein with one or more excipients (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

In an embodiment of the invention, the further therapeutic agent is an oligonucleotide (e.g., DNA or RNA or a duplex of both), e.g., that binds to DNA or mRNA encoding C5 and inhibits C5 expression. In an embodiment of the invention, the oligonucleotide is up to about 23, about 19-22, about 19-23 or about 19, about 20, about 21, about 22 or about 23 nucleotides in length (e.g., a 19-23 nucleotide RNA molecule). In an embodiment of the invention, the oligonucleotide is single stranded (e.g., in anti-sense orientation) or double stranded. A double stranded oligonucleotide includes a strand in sense orientation and a strand in an anti-sense orientation. In an embodiment of the invention, the double stranded oligonucleotide (e.g., RNA) has a 3′ overhang and/or a 5′ overhang, for example, of at least two nucleotides. In an embodiment of the invention, the oligonucleotide is naked and in another embodiment the oligonucleotide is chemically modified.

In an embodiment of the invention, the further therapeutic agent is an oligonucleotide which is an RNAi agent that binds to an RNA encoding C5 or a portion thereof. An RNAi agent refers to an agent that contains RNA and which mediates the targeted cleavage of an RNA transcript via an RNA-induced silencing complex (RISC) pathway. RNAi directs the sequence-specific degradation of mRNA through a process known as RNA interference. The RNAi modulates, e.g., inhibits, the expression of C5 in a cell, e.g., a cell within a subject, such as a mammalian subject.

In one embodiment of the invention, an RNAi agent of the invention includes a single stranded RNA that interacts with a target RNA sequence, e.g., a C5 target mRNA sequence, to direct the cleavage of the target RNA. Without wishing to be bound by theory it is believed that long double stranded RNA introduced into cells is broken down into short-interfering RNA (siRNA) by a Type III endonuclease known as Dicer (Sharp et al. (2001) Genes Dev. 15:485). Dicer, a ribonuclease-III-like enzyme, processes the dsRNA into 19-23 base pair short interfering RNAs (siRNAs) with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., a C5 gene. Accordingly, the term “siRNA” is also used herein to refer to an RNAi as described herein.

In another embodiment, the RNAi agent may be a single-stranded siRNA that is introduced into a cell or organism to inhibit a target mRNA. In an embodiment of the invention, single-stranded RNAi agents bind to the RISC endonuclease, Argonaute 2, which then cleaves the target mRNA. The single-stranded siRNAs are, in an embodiment of the invention, 15-30 nucleotides and are chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In an embodiment of the invention, the oligonucleotide (e.g., RNAi) is conjugated to another molecule such as a sugar, such as an N-acetylgalactosamine (GalNAc) derivative such as

[chemical structure]

In an embodiment of the invention, the oligonucleotide (e.g., RNAi) is conjugated to another molecule as shown in the following schematic:

[chemical structure]

wherein X is O or S.

In an embodiment of the invention, the further therapeutic agent is cemdisiran. In an embodiment of the invention, the further therapeutic agent is a double stranded RNA comprising the anti-sense strand nucleotide sequence:

(SEQ ID NO: 370)
5′-UAUUAUAAAAAUAUCUUGCUUUU-3′;

and/or the sense strand comprises the nucleotide sequence:

(SEQ ID NO: 371)
5′-AAGCAAGAUAUUUUUAUAAUA-3′.

In an embodiment of the invention, the further therapeutic agent is a double-stranded ribonucleic acid (dsRNA) agent for inhibiting expression of complement component C5, wherein said dsRNA agent comprises a sense strand and an antisense strand, wherein the sense strand comprises:

(SEQ ID NO: 372)
5′-asasGfcAfaGfaUfAfUfuUfuuAfuAfaua-3′

and the antisense strand comprises:

(SEQ ID NO: 373)
5′-usAfsUfuAfuaAfaAfauaUfcUfuGfcuususudTdT-3′,

wherein a, g, c and u are 2′-O-methyl (2′-OMe) A, G, C, and U, respectively; Af, Gf, Cf and Uf are 2′-fluoro A, G, C and U, respectively; dT is a deoxy-thymine nucleotide; and s is a phosphorothioate linkage; and wherein said sense strand is conjugated at the 3′-terminus to the ligand:

[chemical structure]

See U.S. Pat. No. 9,249,415.

In an embodiment of the invention, the RNAi is in a pharmaceutical formulation comprising a lipid nanoparticle (LNP). An LNP is a vesicle comprising a lipid layer encapsulating a pharmaceutically active molecule such as RNAi. LNPs are described in, for example, U.S. Pat. Nos. 6,858,225, 6,815,432, 8,158,601, and 8,058,069, the entire contents of which are hereby incorporated herein by reference.

In an embodiment of the invention, the further therapeutic agent is acetaminophen, albumin (e.g., in the form of an infusion), ancrod, an angiotensin-converting enzyme inhibitor, an antibiotic (e.g. an oral antibiotic), a further antibody, an anti-CD20 agent, rituximab, an anti-coagulant, an anti-fungal agent, an antihypertensive, an anti-inflammatory drug, antiplasmin-a1, an anti-seizure agent, anti-thrombotic agent, an anti-TNFalpha agent, an anti-viral agent, argatroban, aspirin, a biological therapeutic agent, bivalirudin, a C3 inhibitor, a corticosteroid, cyclosporine A, dabigatran, defibrotide, E-aminocaproic acid, enteral feeding, erythromycin, erythropoietin, a fibrinolytic agent, folic acid, fondaparinux, heparin, hormone replacement therapy, ibuprofen, idraparinux, an immunosuppressive drug, infliximab, an inhibitor of hydroxymethylglutaryl CoA reductase, an iron supplement, lepirudin, lipid-lowering agent, magnesium sulfate, a Meningococcal vaccine (e.g. serotypes A, C, Y, W and serotype B), methotrexate, a non-steroidal anti-inflammatory drug (NSAID), an oligonucleotide, paracetamol, parenteral feeding, penicillin, phenindione, a pregnancy contraceptive drug, prostacyclin, rituximab, a thrombin inhibitor, a vaccine, vincristine, a vitamin and/or warfarin.

The term “in association with” indicates that components of composition, e.g., including (1) an antagonist antigen-binding protein that binds specifically to C5 and pharmaceutically acceptable carrier components, along with (2) one or more further therapeutic agents, such as cemdisiran, can be formulated into a single composition, e.g., for simultaneous delivery, or formulated separately into two or more compositions (e.g., a kit including each component, for example, wherein the further therapeutic agent is in a separate formulation). Components administered in association with each another can be administered to a subject at the same time or at a different time than when the other component is administered; for example, each administration may be given simultaneously (e.g., together in a single composition or essentially simultaneously during the same administration session) or non-simultaneously at one or more intervals over a given period of time. Moreover, the separate components administered in association with each another may be administered to a subject by the same or by a different route.

### C5 Oligonucleotide Dosage

The present invention provides methods for treating or preventing C5-related disorders, in a subject, by administering an antagonist antigen-binding protein that binds specifically to C5 (e.g., REGN3918), e.g., (i) administering one or more doses of about 30 mg/kg (body weight (BW)) of the antigen-binding protein intravenously (IV); then, optionally, (ii) administering either one or more weekly SC doses of about 800 mg of the antigen-binding protein; or one or more weekly SC doses according to body weight as follows: for body weight (BW)<10 kg: about 125 mg; for BW ≥10 kg and <20 kg: about 200 mg; for BW ≥20 kg and <40 kg: about 350 mg; for BW ≥40 kg and <60 kg: about 500 mg; and for BW ≥60 kg: about 800 mg; wherein, optionally, the subject is further administering a therapeutically effective amount of oligonucleotide that binds to a polynucleotide encoding C5 and inhibits expression of C5 (a C5 oligonucleotide).

A therapeutically effective dose of a dsRNA, RNAi or other oligonucleotide that binds a polynucleotide encoding C5 and inhibits expression of C5 will, in an embodiment of the invention, be in the range of about 0.001 to about 200.0 milligrams per kilogram body weight of the recipient per day, generally in the range of about 1 to 50 mg per kilogram body weight per day. For example, a dsRNA can be administered at about 0.01 mg/kg, about 0.05 mg/kg, about 0.5 mg/kg, about 1 mg/kg, about 1.5 mg/kg, about 2 mg/kg, about 3 mg/kg, about 10 mg/kg, about 20 mg/kg, about 30 mg/kg, about 40 mg/kg, or about 50 mg/kg per single dose.

In an embodiment of the invention, a C5 dsRNA is administered at a dose of about 0.1 to about 20 mg/kg, about 0.1 to about 30 mg/kg, about 0.1 to about 40 mg/kg, about 0.1 to about 45 mg/kg, about 0.1 to about 50 mg/kg, about 0.25 to about 20 mg/kg, about 0.25 to about 30 mg/kg, about 0.25 to about 40 mg/kg, about 0.25 to about 45 mg/kg, about 0.25 to about 50 mg/kg, about 0.5 to about 20 mg/kg, about 0.5 to about 30 mg/kg, about 0.5 to about 40 mg/kg, about 0.5 to about 45 mg/kg, about 0.5 to about 50 mg/kg, about 0.75 to about 20 mg/kg, about 0.75 to about 30 mg/kg, about 0.75 to about 40 mg/kg, about 0.75 to about 45 mg/kg, about 0.75 to about 50 mg/kg, about 1 to about 20 mg/mg, about 1 to about 30 mg/mg, about 1 to about 40 mg/mg, about 1 to about 45 mg/mg, about 1 to about 50 mg/mg, about 1.5 to about 20 mg/kb, about 1.5 to about 30 mg/kb, about 1.5 to about 40 mg/kb, about 1.5 to about 45 mg/kb, about 1.5 to about 50 mg/kb, about 10 to about 20 mg/kg, about 10 to about 30 mg/kg, about 10 to about 40 mg/kg, about 10 to about 45 mg/kg, about 10 to about 50 mg/kg, about 15 to about 20 mg/kg, about 15 to about 30 mg/kg, about 15 to about 40 mg/kg, about 15 to about 45 mg/kg, about 15 to about 50 mg/kg, about 2 to about 20 mg/kg, about 2 to about 30 mg/kg, about 2 to about 40 mg/kg, about 2 to about 45 mg/kg, about 2 to about 50 mg/kg, about 2.5 to about 20 mg/kg, about 2.5 to about 30 mg/kg, about 2.5 to about 40 mg/kg, about 2.5 to about 45 mg/kg, about 2.5 to about 50 mg/kg, about 20 to about 30 mg/kg, about 20 to about 40 mg/kg, about 20 to about 45 mg/kg, about 20 to about 50 mg/kg, about 25 to about 30 mg/kg, about 25 to about 40 mg/kg, about 25 to about 45 mg/kg, about 25 to about 50 mg/kg, about 3 to about 20 mg/kg, about 3 to about 30 mg/kg, about 3 to about 40 mg/kg, about 3 to about 45 mg/kg, about 3 to about 50 mg/kg, about 3.5 to about 20 mg/kg, about 3.5 to about 30 mg/kg, about 3.5 to about 40 mg/kg, about 3.5 to about 45 mg/kg, about 3.5 to about 50 mg/kg, about 30 to about 40 mg/kg, about 30 to about 45 mg/kg, about 30 to about 50 mg/kg, about 35 to about 40 mg/kg, about 35 to about 45 mg/kg, about 35 to about 50 mg/kg, about 4 to about 20 mg/kg, about 4 to about 30 mg/kg, about 4 to about 40 mg/kg, about 4 to about 45 mg/kg, about 4 to about 50 mg/kg, about 4.5 to about 20 mg/kg, about 4.5 to about 30 mg/kg, about 4.5 to about 40 mg/kg, about 4.5 to about 45 mg/kg, about 4.5 to about 50 mg/kg, about 40 to about 45 mg/kg, about 40 to about 50 mg/kg, about 45 to about 50 mg/kg, about 5 to about 20 mg/kg, about 5 to about 30 mg/kg, about 5 to about 40 mg/kg, about 5 to about 45 mg/kg, about 5 to about 50 mg/kg, about 7.5 to about 20 mg/kg, about 7.5 to about 30 mg/kg, about 7.5 to about 40 mg/kg, about 7.5 to about 45 mg/kg, about 7.5 to about 50 mg/kg. Values and ranges intermediate to the recited values are also intended to be part of this invention. In one embodiment, the dsRNA is administered at a dose of about 10 mg/kg to about 30 mg/kg.

For example, the C5 dsRNA or RNAi or other oligonucleotide may be administered e.g., subcutaneously or intravenously, at a dose (or repeated doses) of about 0.01, 0.02, 0.03, 0.04, 0.05, 0.06, 0.07, 0.08, 0.09, 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49 or 50 mg/kg. A multi-dose regimen may include administration of a therapeutic amount of C5 dsRNA or RNAi or other oligonucleotide daily, such as for two days, three days, four days, five days, six days, seven days, or longer. A repeat-dose regimen may include administration of a therapeutic amount of C5 dsRNA or RNAi or other oligonucleotide on a regular basis, such as every other day, every third day, every fourth day, twice a week, once a week, every other week, or once a month.

A C5 dsRNA or RNAi or other oligonucleotide may be administered e.g., subcutaneously or intravenously, at a dose (or repeated doses) of about 600 mg. A pharmaceutical composition including an oligonucleotide can be administered by intravenous infusion over a period of time, such as over a 5, 6, 7, 8, 9, 10, 11, 12, 13, 14, 15, 16, 17, 18, 19, 20, and 21, 22, 23, 24, or about a 25 minute period. The administration may be repeated, for example, on a regular basis, such as weekly, biweekly (i.e., every two weeks) for one month, two months, three months, four months or longer. After an initial treatment regimen, the treatments can be administered on a less frequent basis. For example, after administration weekly or biweekly for three months, administration can be repeated once per month, for six months or a year or longer.

### EXAMPLES

These examples are intended to exemplify the present invention are not a limitation thereof. Compositions and methods set forth in the Examples form part of the present invention.

### Example 1: An Open-Label, Single Arm Study to Evaluate the Efficacy and Safety of REGN3918 in Patients with Paroxysmal Nocturnal Hemoglobinuria (PNH) Who are Complement Inhibitor-Naive or have not Recently Received Complement Inhibitor Therapy

This is an open-label, single arm, 26-week treatment study in patients with confirmed diagnosis of PNH and active signs and symptoms who either are complement inhibitor naïve or have received prior treatment with a complement inhibitor, but not within 6 months prior to the screening visit.

In this study, there will be two cohorts, one for dose confirmation (cohort A) and one for dose expansion (cohort B). The dose confirmation of 30 mg/kg REGN3918 (IV), followed by 800 mg SC weekly was made at the interim analysis. The inclusion and exclusion criteria and schedule of events are the same for cohort A and cohort B. During the assessment of data from cohort A, recruitment into the study will continue. Patients will be given a single loading dose of REGN3918, 30 mg/kg intravenous (IV) on day 1, then a dose not greater than 800 mg subcutaneous (SC) once weekly (QW; ±1 day) to week 26.

The primary objective of the study is to demonstrate a reduction in intravascular hemolysis by REGN3918 over 26 weeks of treatment in patients with active PNH who are treatment-naive to complement inhibitor therapy or have not recently received complement inhibitor therapy. The secondary objectives of the study are to evaluate the safety and tolerability of REGN3918; to evaluate the effect of REGN3918 on parameters of intravascular hemolysis; to assess the concentrations of total REGN3918 in serum; to evaluate the incidence of treatment-emergent anti-drug antibodies to REGN3918; and to evaluate the effect of REGN3918 on patient-reported outcomes (PROs) measuring fatigue and health-related quality of life.

### Study Duration

The duration of the study for a patient is approximately 27 weeks, excluding the screening period. The study consists of a screening period (up to 4 weeks), 26 week treatment period, and an end-of-study visit one week after the last study drug administration. After completion of the 26 week treatment period, patients may enroll into a separate open label extension study, which will provide uninterrupted treatment with REGN3918. Patients who discontinue treatment will have a minimum of a 21 week follow up period.

### Study Population

Approximately 30 to 42 adult men and women will be enrolled. The study population will consist of adult male and female patients with confirmed diagnosis of PNH and active signs and symptoms who either are complement inhibitor-naïve or have received prior treatment with a complement inhibitor, but not within 6 months prior to screening visit.

### Inclusion Criteria

A patient must meet the following criteria to be eligible for inclusion in the study:

1. Male or female ≥18 years of age or legal age of majority at screening, whichever is greater;
2. Diagnosis of PNH confirmed by high sensitivity flow cytometry;
3. PNH granulocytes (denoted as polymorphonuclear [PMN]) >10% at screening visit;
4. Active disease, as defined by the presence of 1 or more PNH related signs or symptoms (e.g., fatigue, hemoglobinuria, abdominal pain, shortness of breath [dyspnea], anemia [hemoglobin <10 g/dL], history of a MAVE (major adverse vascular events) [including thrombosis], dysphagia, or erectile dysfunction) or history of RBC transfusion due to PNH within 3 months of screening;
5. LDH level ≥2×ULN (upper limit of normal) at screening visit;
6. Willing and able to comply with clinic visits and study related procedures;
7. Provide informed consent signed by study patient; and
8. Able to understand and complete study related questionnaires.

### Exclusion Criteria

A patient who meets any of the following criteria will be excluded from the study:

1. Prior treatment with a complement inhibitor either within 6 months prior to screening visit or at any time where the patient was refractory to complement inhibitor therapy, in the opinion of the investigator (with the exception of eculizumab refractory patients due to the C5 variant R885H/C);
2. History of bone marrow transplantation;
3. Body weight <40 kilograms at screening visit;
4. Planned modification (initiation, discontinuation, or dose/dosing interval change) to the following background concomitant medications, as applicable, during screening and treatment periods: erythropoietin, immunosuppressive drugs, corticosteroids, anti-thrombotic agents, anticoagulants, iron supplements, and folic acid;
5. Peripheral blood absolute neutrophil count (ANC)<500/μL [<1.0×10⁹/L] or peripheral blood platelet count <50,000/μL;
6. No documented meningococcal vaccination within 3 years prior to screening and patient unwillingness to undergo vaccination during the study;
7. Documented history of systemic fungal disease or unresolved tuberculosis, or evidence of active or latent tuberculosis infection (LTBI) during screening period. Assessment for active TB and LTBI should accord with local practice or guidelines, including those pertaining to risk assessment, and the use of tuberculin skin test or T-cell interferon-gamma release assay;
8. Any contraindication for receiving Neisseria meningitidis vaccination and antibiotic prophylaxis therapy as recommended in the study;
9. Any active, ongoing infection within 2 weeks of screening or during the screening period;
10. A recent infection requiring ongoing systemic treatment with antibiotics, antivirals, or antifungals within 2 weeks of screening or during the screening period;
11. Immunization with a live attenuated vaccine 1 month prior to REGN3918 administration;
12. Known hereditary complement deficiency;
13. Documented history of active, ongoing systemic autoimmune diseases;
14. Documented history of liver cirrhosis, or patients with liver disease unrelated to PNH with ALT or AST greater than 3×ULN at the screening visit;
15. Patients with an estimated glomerular filtration rate (eGFR) of <30 mL/min/1.73 m² (according to Chronic Kidney Disease Epidemiology Collaboration equation 2009) at screening visit;
16. Recent, unstable medical conditions, excluding PNH and PNH related complications, within the past 3 months prior to screening visit (e.g., myocardial infarction, congestive heart failure with New York Heart Association Class ≥III, serious uncontrolled cardiac arrhythmia, cerebrovascular accident, active gastrointestinal bleed);
17. Anticipated need for major surgery during the study;
18. Coexisting, chronic anemia unrelated to PNH;
19. History of cancer within the past 5 years, except for adequately treated basal cell skin cancer, squamous cell skin cancer, or in situ cervical cancer;
20. Participation in another interventional clinical study or use of any experimental therapy within 30 days before screening visit or within 5 half-lives of that investigational product, whichever is greater, with the exception of complement inhibitors;
21. Known sensitivity to doxycycline or to any of the components of the REGN3918 formulation and drug product;
22. History of significant multiple and/or severe allergies (including latex gloves) or has had an anaphylactic reaction or significant intolerability to prescription or nonprescription drugs;
23. Any clinically significant abnormality identified at the time of screening that in the judgment of the Investigator or any sub-Investigator would preclude safe completion of the study or constrain endpoints assessment such as major systemic diseases, or patients with short life expectancy;
24. Considered by the Investigator or any sub-investigator as inappropriate for this study for any reason, e.g.:
Deemed unable to meet specific protocol requirements, such as scheduled visits
Deemed unable to tolerate long term injections as per the patient, the investigator, sub Investigator, pharmacist, study coordinator, other study staff, or relative thereof directly involved in the conduct of the protocol, etc.
Presence of any other conditions (e.g., geographic, social, etc.), actual or anticipated, that the investigator feels would restrict or limit the patient's participation for the duration of the study;
   25. Women who are pregnant, breastfeeding, or who have a positive pregnancy test at screening visit or day 1;
   26. Patients who are committed to an institution by virtue of an order issued either by the judicial or the administrative authorities;
   27. Pregnant or breastfeeding women;
   28. Women of childbearing potential* who are unwilling to practice highly effective contraception prior to the initial dose/start of the first treatment, during the study, and for at least 21 weeks after the last dose. Highly effective contraceptive measures include:
a. stable use of combined (estrogen and progestogen containing) hormonal contraception (oral, intravaginal, transdermal) or progestogen only hormonal contraception (oral, injectable, implantable) associated with inhibition of ovulation initiated 2 or more menstrual cycles prior to screening
b. intrauterine device (IUD); intrauterine hormone releasing system (IUS)
c. bilateral tubal ligation
d. vasectomized partner
e. and/or sexual abstinences^{†‡}; * Postmenopausal women must be amenorrheic for at least 12 months in order not to be considered of childbearing potential. Pregnancy testing and contraception are not required for women with documented hysterectomy or tubal ligation.^{†} Sexual abstinence is considered a highly effective method only if defined as refraining from heterosexual intercourse during the entire period of risk associated with the study treatments. The reliability of sexual abstinence needs to be evaluated in relation to the duration of the clinical trial and the preferred and usual lifestyle of the subject.^{‡} Periodic abstinence (calendar, symptothermal, post ovulation methods), withdrawal (coitus interruptus), spermicides only, and lactational amenorrhea method (LAM) are not acceptable methods of contraception. Female condom and male condom should not be used together;
   or
   29. Known chronic infection with hepatitis B or C, defined as a testing history showing a currently positive status for hepatitis B surface antigen (HBsAg), hepatitis B e antigen (HBeAg), hepatitis B virus DNA, or hepatitis C virus RNA (HCV RNA).

### Endpoints

The co-primary endpoints are:
The proportion of patients achieving adequate control of their intravascular hemolysis, defined as LDH ≤1.5×ULN at every scheduled time point between week 4 and week 26, inclusive; and
The proportion of patients achieving transfusion avoidance defined as no post-baseline transfusion of RBCs per protocol through week 26.

The secondary endpoints are:
The rate of breakthrough hemolysis through week 26, defined as the measurement of LDH ≥2×ULN concomitant with associated signs or symptoms at any time subsequent to an initial achievement of disease control (i.e., LDH ≤1.5×ULN);
The proportion of patients achieving normalization of their intravascular hemolysis, defined as LDH ≤1.0×ULN at every scheduled time point between week 4 through week 26, inclusive;
Time to first LDH ≤1.5×ULN;
Percentage of days with LDH ≤1.5×ULN between week 4 and week 26, inclusive;
Change and percent change in LDH levels from baseline to week 26;
The rate and number of units of transfusion with RBCs through week 26;
Change in RBC hemoglobin levels from baseline to week 26;
Change in free hemoglobin levels from baseline to week 26;
Change and percent change in CH50 from baseline to week 26;
Change in patient-reported outcomes (FACIT-Fatigue, European Organization for Research and Treatment of Cancer [EORTC]-QLQ-30, and EQ-5D-3L) from baseline to week 26;
Incidence and severity of treatment-emergent adverse events (TEAEs) and other safety variables over 26 weeks;
Concentrations of total REGN3918 in serum assessed throughout the study; and
Incidence of treatment-emergent anti-drug antibodies to REGN3918 in patients over time

### Efficacy Measures/Procedures

Serum Lactate Dehydrogenase (LDH). Samples for LDH testing will be collected at visits. Serum LDH levels will the measured in a central lab. On days when Blood

Chemistry is run, then LDH will be included in the panel which will also be run in a central lab. For patients who self-administer, samples may be drawn at home by the visiting nurse when scheduled on non-clinic visits.

Transfusion Record Update. Patients will be requested to provide updated information about the history of transfusions received 1 year prior to the time of screening. During the study, the rate and number of units of transfusion with RBCs will be recorded in the case report form (CRF). Transfusions with RBCs during the study should follow the algorithm described herein. The rate and number of units of transfusion with RBCs will be recorded in the CRF. Hemoglobin levels pre- and post-transfusion will be obtained (including local values).

Total Hemolytic Complement Activity. Samples for CH50 testing will be collected at visits. Serum CH50 levels will the measured in a central lab. For patients who self-administer, samples may be drawn at home by the visiting nurse when scheduled on non-clinic visits.

Red Blood Cell Hemoglobin. Red blood cell hemoglobin testing will be measured in a safety hematology panel collected at visits and will be run in a central lab.

Free Hemoglobin. Free hemoglobin testing will be measured in the safety hematology panel collected at visits and will be run in a central lab.

Clinical Outcome Assessments. The COAs are patient self-reported. Clinical outcome assessments (COAs) will include the Functional Assessment of Chronic Illness Therapy-Fatigue (FACIT-Fatigue) and 2 health-related quality of life (HRQoL) questionnaires (EORTC quality of life questionnaire-core 30 [QLQ-C30] and the EQ-5D-3L) and Patient Global Impression of Severity (PGIS)/Patient Global Impression of Change (PGIC).

The FACIT Fatigue is a 13 item, self-reported PRO measure assessing an individual's level of fatigue during their usual daily activities over the past week. This questionnaire is part of the FACIT measurement system, a compilation of questions measuring health related QoL in patients with cancer and other chronic illnesses. The FACIT Fatigue assesses the level of fatigue using a 4 point Likert scale ranging from 0 (not at all) to 4 (very much). Scores range from 0 to 52, with higher scores indicating greater fatigue. Although the FACIT Fatigue was originally developed to assess fatigue in patients with cancer, it has been used in trials evaluating the efficacy of eculizumab. The FACIT Fatigue has demonstrated content validity among patients with PNH. (Brodsky et al., Multicenter phase 3 study of the complement inhibitor eculizumab for the treatment of patients with paroxysmal nocturnal hemoglobinuria. Blood 2008; 111(4):1840-7; Hillmen et al., The complement inhibitor eculizumab in paroxysmal nocturnal hemoglobinuria. N Engl J Med 2006; 355(12):1233-43.). The FACIT Fatigue has demonstrated content validity among patients with PNH (Weitz et al., Cross-sectional validation study of patient-reported outcomes in patients with paroxysmal nocturnal haemoglobinuria. Intern Med J 2013; 43(3):298-307).

The EORTC QLQ C30 is a 30 item, generic questionnaire commonly used to assess HRQoL in patients with cancer (Stead et al., Development of an EORTC questionnaire module to be used in health-related quality-of-life assessment for patients with multiple myeloma. European Organization for Research and Treatment of Cancer Study Group on Quality of Life. Br J Haematol 1999; 104(3):605-11; Cocks et al., An international field study of the reliability and validity of a disease-specific questionnaire module (the QLQ-MY20) in assessing the quality of life of patients with multiple myeloma. Eur J Cancer 2007; 43(11):1670-8). The EORTC QLQ C30 assesses HRQoL across multiple domains, including global health status, global quality of life, functioning (physical, role, emotional, cognitive, and social functioning), symptom scales (fatigue, nausea and vomiting, pain, appetite loss), and single items (dyspnea, insomnia, constipation, diarrhea, sleep, financial impact). Although the EORTC QLQ 30 was originally developed to assess HRQoL in patients with cancer, it has been used in trials evaluating the efficacy of eculizumab. The EORTC QLQ also has demonstrated content validity among patients with PNH (Brodsky et al., Multicenter phase 3 study of the complement inhibitor eculizumab for the treatment of patients with paroxysmal nocturnal hemoglobinuria. Blood 2008; 111(4):1840-7; Hillmen et al., The complement inhibitor eculizumab in paroxysmal nocturnal hemoglobinuria. N Engl J Med 2006; 355(12):1233-43). The EORTC QLQ also has demonstrated content validity among patients with PNH (Weitz et al., Cross-sectional validation study of patient-reported outcomes in patients with paroxysmal nocturnal haemoglobinuria. Intern Med J 2013; 43(3):298-307.).

The EQ-5D-3L is a self-administered, generic standardized health status measure, consisting of 6 questions. The EQ-5D-3L descriptive system assesses 5 dimensions of health: mobility, self-care, usual activities, pain/discomfort, and anxiety/depression. Each dimension is rated on a 3-level scale: no problems, some problems, and extreme problems. The EQ visual analog scale component is a vertical, visual analog scale used by patients to rate their health.

Patient Global Impression of Severity/Patient Global Impression of Change. Patient Global Impression of Severity consists of 3 self-administered PRO questions assessing the patient's perception of the overall severity of the symptoms of their disease and/or of a specific symptom of their disease. At study visits, patients will be asked to rate the severity of their PNH symptoms on a 6-point Likert scale ranging from “I am not experiencing PNH symptoms” to “very severe”; the impact their PNH symptoms have on their ability to perform usual daily activities on a 5-point Likert scale ranging from “not at all impacted” to “extremely impacted”; and their overall fatigue on a 5-point Likert scale ranging from “not fatigued” to “extremely fatigued”.

Patient Global Impression of Change consists of 3 self-administered PRO questions assessing the patient's perception of the change in overall severity of the symptoms of their disease and/or of a specific symptom of their disease compared to the start of the study. At key time points during the study, patients will be asked to rate the change in PNH symptoms, in their ability to perform usual daily activities, and in overall fatigue compared to before the start of the study on a 7-point Likert scale ranging from “much better” to “no change” to “much worse”.

PGIS and PGIC questions are developed for this trial and allow for the interpretation of PRO findings and the investigation of a responder definition. The answers on the PGIS and PGIC items serve as “anchors” to help interpret the mean change in disease-specific PRO measures over time and to estimate responder definitions. This empirical anchor-based approach is the primary FDA-recommended approach for defining a responder and analyzing responder-based PRO results.

### Study Design

This is an open label, single arm, 26 week treatment study in patients with confirmed diagnosis of PNH and active signs and symptoms who either are complement inhibitor naive or have received prior treatment with a complement inhibitor, but not within 6 months prior to screening visit.

In this study, there will be two cohorts, one for dose confirmation (cohort A) and one for dose expansion (cohort B). Dose confirmation will be made at the interim analysis. The inclusion and exclusion criteria and schedule of events are the same for cohort A and cohort B. During the assessment of data from cohort A, recruitment into the study will continue, with patients recruited being assigned subsequently as follows: if a decision is made to expand cohort A, they will be assigned to cohort A. If a decision is made to progress to cohort B, they will be assigned to cohort B.

Patients will be given a single loading dose of REGN3918 30 mg/kg intravenous (IV) on day 1, then a dose not greater than 800 mg subcutaneous (SC) once weekly (QW; ±1 day) to week 26.

### Dosage

A single loading dose of REGN3918 30 mg/kg IV on day 1, then 800 mg SC once weekly (QW), has been initially selected. A minimum concentration of 100 mg/L REGN3918 is required to maximally suppress C5 activity. The loading dose of 30 mg/kg IV will help to quickly achieve the steady-state trough concentration required for sustained maximal CH50 inhibition.

### Pharmacokinetics (PK)

The PK variable is the concentration of total REGN3918 at each time point. The sampling time points are specified in Table 1-1.

### Anti-Drug Antibody (ADA)

The anti-drug antibody (ADA) variables are ADA status, titer, and time point/visit. Samples in this study will be collected at the clinic visits specified in Table 1-1. Blood samples for ADA assessment in serum will be collected prior to drug administrations.

### Study Cohorts

In this study, there will be two cohorts, one for dose confirmation (cohort A) and one for dose expansion (cohort B). Dose confirmation will be made at the interim analysis. The inclusion and exclusion criteria and schedule of events are the same for cohort A and cohort B. During the assessment of data from cohort A, recruitment into the study will continue, with patients recruited being assigned subsequently as follows: If a decision is made to expand cohort A, they will be assigned to cohort A. If a decision is made to progress to cohort B, they will be assigned to cohort B. A study flow diagram depicting the treatment of each cohort is set forth in FIG. 1.

At the time of the decision, other relevant available data may be considered as part of the decision-making process, including clinical data, REGN3918 PK (as available), CH50, total C5, the LDH level achieved in those not achieving ≤1.5×ULN, and safety.

The decision to progress from cohort A to cohort B will be made by the Sponsor in conjunction with the global principal investigator based on the achievement of LDH reduction to <1.5×ULN and safety at week 8, as follows:
If all 6 out of 6 cohort A patients achieve an LDH ≤1.5×ULN at week 8 and the dosing regimen is considered well tolerated, then either the dose regimen will be confirmed and the study will progress to cohort B, or the dosing regimen will be altered, with a lower dose and/or longer dosing interval being tested in an expanded cohort A (up to a further 6 subjects). These revisions would not be considered substantial and therefore would not require a formal protocol amendment.
If one or more patients fails to achieve LDH ≤1.5×ULN at week 8, then after consideration of all data (including clinical and safety data, REGN3918 PK, CH50, total C5, baseline LDH and the LDH level achieved), a decision will be made to either:
   Confirm the dosing regimen and progress to cohort B, or
   Continue with the selected dosing regimen and expand cohort A up to a maximum of 12 patients, or
   Increase dose and/or reduce dose interval and re-assess cohort A. This option will require a substantial protocol amendment.

After the first administration of REGN3918 at the study site, subsequent administrations may either be continued at the clinical site, or by the site personnel or another healthcare professional at patient's home (if possible), or self-administered/administered by the patient or designated person, respectively.

### Drug Administration

Patients will be given a single loading dose of REGN3918 30 mg/kg IV on day 1, then a dose no greater than 800 mg SC QW (±1 day) over the treatment period. The weekly subcutaneous dose is 800 mg SC QW (±1 day) for the initial cohort A patients.

For cohort A, after the IV loading dose administration of REGN3918 at the study site, subsequent SC administrations may either be continued at the clinical site by the site personnel or at the patient's home by another healthcare professional. After week 8, self-administration/administration by the patient or designated person may occur.

For cohort B, subsequent administrations may be continued at the clinic site, at the patient's home by another healthcare professional, or by the patient/designated person. The location and administration options for SC route of administration will depend on the preference of the investigator and patient (e.g., abdomen, thigh, or upper arm), the availability of clinical supply, and home healthcare visiting professional. Clinic visits for SC administration may or may not be needed.

If self administration/administration by patient/designated person is allowed locally, then sufficient injection training at the scheduled injection with REGN3918 will be provided. After training, observation of self administration/administration by patient/designated person will be conducted by clinical site personnel or visiting healthcare professional. Once this observation is considered satisfactory, then the study drug can be subsequently administered independently by patient/designated person for the remainder of the study. In addition, a patient diary will be provided prior to initiation of self administration (i.e., week 8 for cohort A and week 4 for cohort B). The diary should be completed upon each the study drug administration. A study drug kit will be dispensed at clinical site visit, using a direct to patient (DTP) service provider, or transported by a healthcare professional, as applicable.

### Red Blood Cell (RBC) Transfusions

Transfusions with RBCs during the study should proceed according to the following predefined criteria that will trigger a transfusion; however, the actual number of units to be transfused is at the discretion of the investigator:
Transfuse with RBC(s) if the post baseline hemoglobin level is <9 g/dL with symptoms resulting from anemia; or
Transfuse with RBC(s) if the post baseline hemoglobin level is <7 g/dL.

### Pre-Treatments

Enrolled patients will require evidence of meningococcal immunization or administration of vaccination during the screening period and oral antibiotics are recommended during the treatment period, according to local practice.

### Dose Modification and Study Treatment Discontinuation Rules

Dose modification for an individual patient is not allowed. Patients who permanently discontinue from study drug and who do not withdraw from the study will be asked to return to the clinic for all remaining study visits. Patients who permanently discontinue from study drug and who opt to withdraw from the study will be asked to complete study assessments. Study drug dosing will be permanently stopped in the event of:
Evidence of pregnancy;
Serious or severe allergic reactions considered related to study drug;
Liver impairment as evidenced by one or more of the following criteria:
   Alanine aminotransferase (ALT) or aspartate aminotransferase (AST) >8× ULN; or
   ALT or AST >5×ULN for more than 2 weeks; or
   ALT or AST >3×ULN and total bilirubin >2×ULN (or international normalized ratio (INR) >1.5) and no other reason can be found to explain the combination of increased AST/ALT and total bilirubin, such as viral hepatitis A, B, or C; pre-existing or acute liver disease; or another drug capable of causing the observed injury;
Patient withdrawal of consent;
Patient non-compliance (e.g., not complying with protocol required visits, assessments, and/or dosing instructions); or
Investigator's clinical judgment that it is in the best interest of the patient.

Temporary discontinuation may be considered by the Investigator because of suspected AEs. The investigator can reinitiate treatment with study drug under close and appropriate clinical and/or laboratory monitoring once the Investigator will have considered according to his/her best medical judgment that the responsibility of the study drug in the occurrence of the concerned event was unlikely.

### Acute Reactions

Patients should be observed for 30 minutes after the IV infusion. Emergency equipment and medication for the treatment of infusion reactions must be available for immediate use. All infusion reactions must be reported as AEs and graded. The infusion should be interrupted if any of the following AEs are observed: cough, rigors/chills, rash, pruritus (itching), urticaria (hives, welts, wheals), diaphoresis (sweating), hypotension, dyspnea (shortness of breath), vomiting, flushing. The reaction(s) should be treated symptomatically, and the infusion may be restarted at 50% of the original rate. If investigators feel there is a medical need for treatment or discontinuation of the infusion other than described above, they should use clinical judgment to provide the appropriate response according to typical clinical practice.

The infusion should be terminated and NOT restarted if any of the following AEs occur: anaphylaxis^{⋅}, laryngeal/pharyngeal edema, severe bronchospasm, chest pain, seizure, severe hypotension, other neurological symptoms (confusion, loss of consciousness, paresthesia, paralysis, etc); or any other symptom or sign that, in the opinion of the investigator, warrants termination of the IV infusion * Consider anaphylaxis if the following is observed (Sampson et al., Second symposium on the definition and management of anaphylaxis: summary report—Second National Institute of Allergy and Infectious Disease/Food Allergy and Anaphylaxis Network symposium. J Allergy Clin Immunol 2006; 117(2):391-7): acute onset of an illness (minutes to several hours) with involvement of the skin, mucosal tissue, or both (e.g., generalized hives, pruritus or flushing, swollen lips tongue uvula) and at least one of the following: respiratory compromise (e.g., dyspnea, wheeze bronchospasm, stridor, reduced peak expiratory flow, hypoxemia); or reduced blood pressure or associated symptoms of end organ dysfunction (e.g., hypotonia [collapse], syncope, incontinence).

Patients should be observed for 30 minutes after the first SC injection. Emergency equipment and medication for the treatment of systemic reactions must be available for immediate use at the site. All injection reactions must be reported as AEs and graded. Acute systemic reactions following injection of study drug (SC) should be treated using clinical judgment to determine the appropriate response according to typical clinical practice. Local injection site reactions must be reported as AEs and graded.

### Concomitant Medications

Any treatment administered from the time of informed consent to the end of final study visit will be considered concomitant medication. This includes medications that were started before the study and are ongoing during the study.

The following medications are prohibited, with the exception of those listed below:
Within 24 hours prior to each clinic visit when blood is drawn, patients should not consume any alcohol;
Beginning on day 1 and continuing throughout the study, while the patient is continuing REGN3918, the patient should not take any other complement inhibitor therapy.

The following medications and procedures will be permitted, under the following conditions:
Any medication required to treat an AE, including systemic corticosteroids, at the discretion of the investigator;
Meningococcal vaccination;
Oral antibiotic prophylaxis;
Oral contraceptives and hormone replacement therapy may continue;
Acetaminophen/paracetamol, aspirin, or ibuprofen at the recommended dose per the local label;
Erythropoietin, immunosuppressive drugs, corticosteroids, anti-thrombotic agents, anticoagulants, iron supplements, and folic acid are permitted and if possible, should be kept constant throughout the study; any changes to these concomitant medications will be at the discretion of the investigator and consistent with practice prior to enrollment;
   and
Any medication required for the treatment of patient's background medical conditions.

TABLE 1-1
Schedule of Events

| Study Period | | | | | | | | | | | | |
|---|---|---|---|---|---|---|---|---|---|---|---|---|
| Screening | Treatment | | | | | | | | | | | EoS |
| Study Week | | | | | | | | | | | | |
| | | | | | | Week 4 to 23 | | | | | | |
| | | | | | | (W 4, 8, | (W 5, 9, | (W 6, 10, | (W 7, 11, | | | |
| up to −4 | 0 | | 1 | 2 | 3 | 12, 16, 20) | 13, 17, 21) | 14, 18, 22) | 15, 19, 23) | 24 | 25 | 26 |
| Study Day (W 0 to W 26 ± 1 D) | | | | | | | | | | | | |
| | | | | | | 29, 57, 85, | 36, 64, 92, | 3, 71, 99, | 50, 78, 106, | | | |
| up to −28 | 1 | 3 | 8 | 15 | 22 | 113, 141 | 120, 148 | 4 127, 155 | 134, 162 | 169 | 176 | 183 |

VISIT LOCATION

| | | | | | | | | | | | | | |
|---|---|---|---|---|---|---|---|---|---|---|---|---|---|
| Clinic Visit | X | X | X | X | X | X | X | | | | X | | X |
| Clinic, Home Healthcare, | | | | | | | | X | X² | X | | X² | |
| or Phone Call Visit¹ | | | | | | | | | | | | | |

SCREENING

| | | |
|---|---|---|
| Informed Consent | X | |
| Inclusion/Exclusion | X | X |
| Medical History | X | X |
| Demographics | X | |
| Prior Concomitant | X | |
| Medications | | |
| Historical Lab | X | |
| Parameters for | | |
| Hemolysis | | |
| Neisseria Meningitidis | X | |
| Vaccination History | | |
| TB history and | X | |
| assessment | | |
| Testing history for | X | |
| hepatitis B and C | | |
| Risk assessment for | X | |
| Neisseria gonorrhea | | |
| Height | X | |
| Enrollment | | X |

TREATMENT

| | | | | | | | | | | | | | |
|---|---|---|---|---|---|---|---|---|---|---|---|---|---|
| R3918 IV Administration | | X | | | | | | | | | | | |
| R3918 SC Administration | | | | X | X | X | X | X | X | X | X | X | |
| Oral Antibiotics | | X | X | X | X | X | X | X | X | X | X | X | X |
| Meningococcal Vaccine | X | X | | | | | | | | | | | |
| IVRS/IWRS Contact | X | X | X | X | X | X | X | X | X | X | X | X | X |
| Patient Diary | | | | | | | | X | X | X | | X | |

EFFICACY (CENTRAL)

| | | | | | | | | | | | | | |
|---|---|---|---|---|---|---|---|---|---|---|---|---|---|
| Serum LDH | X | X | X | X | X | X | X | | X | | X | X | X |
| Transfusion Data | X | X | X | X | X | X | X | X | X | X | X | X | X |
| Complement Hemolytic | X | X | X | X | X | X | X | | | | | | X |
| Assay (serum CH50) | | | | | | | | | | | | | |
| FACIT-Fatigue | | X | | | X | | X | | | | | | X |
| EORTC-QLQ-30 | | X | | | X | | X | | | | | | X |
| EQ-5D-3L | | X | | | | | X | | | | | | X |
| PGIS | | X | | | X | | X | | | | | | X |
| PGIC | | | | | X | | X | | | | | | X |

SAFETY

| Vital Signs | X | X | X | X | X | X | X | | X | | | | X |
|---|---|---|---|---|---|---|---|---|---|---|---|---|---|
| Physical Examination | X | X | X | X | | | X | | | | | | X |
| Body Weight | X | X | | | | | X | | | | | | X |
| Electrocardiogram | X | | | | | | | | | | | | X |
| Adverse Events | < -------------------------------------------------------x ----------------------------------------------------------- > | | | | | | | | | | | | |
| | If breakthrough hemolysis is suspected, blood samples will be collected for LDH and other assessments. | | | | | | | | | | | | |
| | Blood may also be collected in the event of drug hypersensitivity | | | | | | | | | | | | |
| Concomitant Meds & Tx | X | X | X | X | X | X | X | X | X | X | X | X | X |
| Patient Safety Card | | X | | | | | | | | | | | |

LABORATORY TESTING: (CENTRAL)

| | | | | | | | | |
|---|---|---|---|---|---|---|---|---|
| Hematology | X | X | | | X | | X | X |
| (Includes RBC and | | | | | | | | |
| free hemoglobin) | | | | | | | | |
| Blood Chemistry | X | X | | | X | | X | X |
| (Includes serum | | | | | | | | |
| lactate dehydrogenase) | | | | | | | | |
| Pregnancy Test | X | X | | | X | | X | X |
| Urinalysis | X | X | | | X | | X | X |
| C-Reactive Protein | X | X | X | X | X | X | X | X |
| Direct Anti-globulin | X | X | | | | | X | X |
| Test | | | | | | | | |

PK AND ADA SAMPLES

| | | | | | |
|---|---|---|---|---|---|
| PK Sample (Day 1, | X | X | X | X | X |
| sample pre dose | | | | | |
| and end of the IV | | | | | |
| infusion. Pre dose at | | | | | |
| all other visits.) | | | | | |
| ADA Sample (Pre-dose) | X | | | X (W 12) | X |

EXPLORATORY PD/BIOMARKERS

| | | | | | | | | |
|---|---|---|---|---|---|---|---|---|
| Haptoglobin | X | X | | | | | X | X |
| Reticulocyte Count | X | X | | | | | X | X |
| Complement Hemolytic Assay | X | X | X | X | X | X | X | X |
| (serum AH50) (Day 1, | | | | | | | | |
| sample pre dose | | | | | | | | |
| and end of the IV | | | | | | | | |
| infusion. Pre dose at | | | | | | | | |
| all other visits.) | | | | | | | | |
| Total Complement Levels | X | X | | | | | X | X |
| Total C5 (plasma) | X | X | X | | | | X | X |
| (Day 1, sample pre dose | | | | | | | | |
| and end of the IV | | | | | | | | |
| infusion. Pre dose at | | | | | | | | |
| all other visits) | | | | | | | | |
| C5a (plasma and urine) | X | X | | | | | X | X |
| sC5b-9 (plasma) | X | X | X | X | X | X | X | X |
| PNH Erythrocyte Cells | X | X | | | | | X (W 8, | X |
| | | | | | | | W 16) | |
| PNH Granulocyte Cells | X | X | | | | | X (W 8, | X |
| | | | | | | | W 16) | |
| NT-proBNP | X | X | | | | | X | X |
| Biomarkers of Thrombosis | X | X | | | | | X | X |
| and Inflammation | | | | | | | | |
| Renal Injury Markers | X | X | | | | | X | X |

OPTIONAL SAMPLES

| | | | | |
|---|---|---|---|---|
| Future Biomedical | X | | X | X |
| Research Serum/Plasma | | | | |
| (optional) | | | | |
| Whole Blood for DNA | X | | | |
| Isolation (optional) | | | | |
| Whole Blood for RNA | X | X | | X |
| Isolation (optional) | | | | |

CLINICAL OUTCOME ASSESSMENTS

| | | | | | | | | | | | | | |
|---|---|---|---|---|---|---|---|---|---|---|---|---|---|
| PNH Symptom-Specific | X | X | X | X | X | X | X | X | X | X | X | X | X |
| Questionnaire | | | | | | | | | | | | | |
| TSQM | | | | | | | X (W 4, | | | | | | X |
| | | | | | | | W 16) | | | | | | |

| | |
|---|---|
| Wearable Device | < ------------------------------ W 0 to W 12 ------------------------------------- > |

EORTC: European Organization for Research and Treatment of Cancer
QLQ C30: Quality of life questionnaire core 30
PGIS: Patient global impression of severity
PGIC: Patient global impression of change
EQ 5D 3L: Euroquol EQ-5D 3 level questionnaire

### Results

The dose (REGN3918, 30 mg/kg intravenous (IV) on day 1, then 800 mg subcutaneous (SC) once weekly (QW; ±1 day)) was confirmed based only on the first 6 subjects through week 8. The study is open label and so LDH will continue to be monitored in all subjects as this is a marker for understanding if patients are experiencing breakthrough hemolysis.

All 6 patients achieved LDH ≤1.5×ULN by Day 15 and remained so through Day 57 (FIG. 5, FIG. 6 and FIG. 7). Median LDH levels and LDH levels in individual subjects (normal scale and semi-log scale) past day 57 are set forth in FIG. 17, FIG. 18, FIG. 19, FIG. 20, FIG. 21, FIG. 22, FIG. 23 and FIG. 24. FIG. 21, FIG. 22, FIG. 23 and FIG. 24 reflect LDH values for 9 patients. All 6 patients were normalized at and after Day 29, except that one patient had LDH=1.01 at Day 43 from 0.89 at Day 29, then returned back to 0.88 at Day 57; and one patient had LDH=1.19 at Day 43 from 0.90 at Day 29, and then returned back to 0.91 at Day 57 (FIG. 8 and FIG. 9). See also, Table 1-2.

TABLE 1-2
Summary of LDH by Visit day

| | Study Day | | | | | | | |
|---|---|---|---|---|---|---|---|---|
| | 1 | 3 | 8 | 15 | 22 | 29 | 43 | 57 |
| LDH (ULN) | 5.48 | 3.25 | 1.70 | 0.95 | 0.85 | 0.73 | 0.87 | 0.74 |
| Mean (SE) | (1.11) | (0.84) | (0.10) | (0.05) | (0.08) | (0.05) | (0.09) | (0.05) |
| LDH (ULN) | 2.67- | 2.03- | 1.48- | 0.80- | 0.64- | 0.60- | 0.62- | 0.62- |
| Range | 10.18 | 7.36 | 2.09 | 1.16 | 1.14 | 0.90 | 1.19 | 0.91 |
| patients | 0 | 0 | 2 | 6 | 6 | 6 | 6 | 6 |
| LDH ≤ 1.5 × | (0%) | (0%) | (33%) | (100%) | (100%) | (100%) | (100%) | (100%) |
| ULN n (%) | | | | | | | | |
| patients | 0 | 0 | 0 | 4 | 5 | 6 | 4 | 6 |
| LDH ≤ 1.0 × | (0%) | (0%) | (0%) | (67%) | (83%) | (100%) | (67%) | (100%) |
| ULN n (%) | | | | | | | | |

A 51 year old Asian female patient with past medical history of PNH, aplastic anemia and prior transfusion of 2 units of RBCs in the past one year screened for the study. The patient had an RBC transfusion of 2 units on Day 50 of the study due to an AE of symptomatic anemia that started on Day 50 and recovered on Day 56. The pre-transfusion HB (hemoglobin) was 7.8 g/dL. The last available HB after transfusion was 11.8 g/dL on Day 57. This transfusion is considered to be a per protocol transfusion.

There were no observed SAEs (serious adverse events), AESIs (adverse events of special interest), or infusion reactions. See Table 1-3.

TABLE 1-3
Treatment Emergent Adverse Event Summary

| System Organ Class | REGN 3918 |
|---|---|
| MedDRA Preferred Term | (N = 6) |
| Number of TEAEs | 9 |
| Number of subjects with | 4 (66.7%) |
| at least one TEAE | |
| Nervous system disorders | 2 (33.3%) |
| Headache | 2 (33.3%) |
| Blood and lymphatic | 1 (16.7%) |
| system disordets | |
| Anaemia | 1 (16.7%) |
| Eye disorders | 1 (16.7%) |
| Conjunctival deposit | 1 (16.7%) |
| Gastrointestinal disorders | 1 (16.7%) |
| Nausea | 1 (16.7%) |
| General disorders and | 1 (16.7%) |
| administration site condition | |
| Injection site reaction | 1 (16.7%) |
| Infections and infestations | 1 (16.7%) |
| Nasopharyngitis | 1 (16.7%) |
| Injury, poisoning and | 1 (16.7%) |
| procedural complications | |
| Foot fracture | 1 (16.7%) |
| Skin and subcutaneous | 1 (16.7%) |
| tissue disorder | |
| Pruritus | 1 (16.7%) |

The serum concentration of REGN3918 was also evaluated in the subjects. See FIG. 9 and FIG. 10. Individual subjects' and median REGN3918 serum concentrations over time are shown in normal scale as well as in semi-log scale (FIGS. 9A, 9B, 10A and 10B). These data are also broken down by gender (FIGS. 9C, 9D, 10C and 10D).

The level of total C5 in subjects administered the REGN3918 was observed to increase over time. See FIG. 11, FIG. 12, FIG. 13, FIG. 14 and FIG. 15. Individual subjects' and the median total C5 concentration over time is shown in FIG. 11 (A-B). These data are also broken down by gender (FIG. 12 (A-B)). The median fold-increase in total C5 over baseline over time and the increases in individual subjects is set forth in FIG. 13 (A-B). These data are also broken down by gender (FIG. 14 (A-B)).

A similar PK/total C5 ratio at steady-state across all 6 patients was observed. The median of the ratio was 4.02. FIG. 16 (A-F).

The data set forth in the present example provides evidence that REGN3918 has properties which are advantageous over that of ALXN1210 and eculizumab. Though the data herein is from only 6 patients, 100% (6 out of 6) of the patients normalized their LDH serum levels. In the studies set forth in FIG. 25, only about half of the patients on ALXN1210 or eculizumab achieved LDH normalization.

### Example 2: A Randomized, Double-Blind, Placebo-Controlled Phase 1 Study of the Pharmacokinetics and Pharmacodynamics of REGN3918, a Human Antibody Against Complement Factor C5, in Healthy Volunteers

REGN3918 (pozelimab), is a fully human monoclonal immunoglobulin antibody directed against the terminal complement protein C5 which inhibits terminal complement activation by blocking C5 cleavage, thereby blocking the formation of the membrane attack complex (MAC; C5b-9). REGN3918 binds with high affinity to wild-type and variant (R885H/C) human C5. REGN3918 was well-tolerated in monkey toxicology studies with up to 26 weeks of dosing at up to 100 mg/kg/wk. This finding was supportive of conducting this first-in-human (FIH) study of REGN3918 in healthy volunteers.

The primary objective of this study was to evaluate the safety and tolerability of REGN3918 administered in healthy volunteers, using both single ascending IV and SC doses and a multiple dose regimen consisting of an IV loading dose plus multiple weekly SC doses. The secondary objectives of this study were to assess the pharmacokinetic and pharmacodynamic profile of REGN3918.

A total of 57 subjects were randomized (56 received study treatment) to 4 sequential ascending IV dose cohorts plus 2 sequential ascending SC cohorts, followed by 1 multiple dose cohort (consisting of an IV loading dose and weekly SC doses). Each cohort consisted of 8 subjects randomized to receive REGN3918 or placebo (6 active: 2 placebo). REGN3918 was administered as follows:
Cohort 1: 1 mg/kg IV, single dose
Cohort 2a: 3 mg/kg IV, single dose
Cohort 2b: 300 mg SC, single dose
Cohort 3a: 10 mg/kg IV, single dose
Cohort 3b: 600 mg SC, single dose
Cohort 4: 30 mg/kg IV, single dose
Cohort 5: Loading dose of 15 mg/kg IV followed by 4 repeat SC doses of 400 mg administered once weekly for 4 weeks.
   See Table 2-1 below.

An adaptive design was implemented to allow for dose level and dosing interval adjustment utilizing in-study pharmacokinetic and pharmacodynamic measures. The pharmacodynamic profile of REGN3918 was assessed utilizing a sheep red blood cell complement activity assay (CH50 assay) as well as serum concentrations of total C5.

TABLE 2-1
Summary of Demographic and Baseline Characteristics of Subjects
by REGN3918 Dose Regimen and Route of Administration

| | | | | | | | | 15 mg/kg |
|---|---|---|---|---|---|---|---|---|
| | Placebo^{a} | 1 mg/kg | 3 mg/kg | 10 mg/kg | 30 mg/kg | 300 mg | 600 mg | IV + 400 mg |
| | (n = 14) | IV(n = 6) | IV (n = 6) | IV (n = 6) | IV (n = 6) | SC (n = 6) | SC (n = 6) | SC^{b} (n = 6) |
| Age, years, | 36.5 | 35.5 | 36.7 | 39.3 | 35.3 | 24.5 | 32.5 | 40.0 |
| mean (SD) | (8.9) | (7.1) | (10.7) | (12.1) | (12.3) | (4.1) | (9.1) | (6.8) |
| Sex, male, n | 9 | 2 | 3 | 3 | 1 | 3 | 2 | 2 |
| (%) | (64.3) | (33.3) | (50.0) | (50.0) | (16.7) | (50.0) | (33.3) | (33.3) |

Race, n (%)

| | | | | | | | | |
|---|---|---|---|---|---|---|---|---|
| White | 12 | 4 | 4 | 5 | 6 | 5 | 4 | 5 |
| | (85.7) | (66.7) | (66.7) | (83.3) | (100) | (83.3) | (66.7) | (83.3) |
| Black or | 2 | 1 | 1 | 1 | 0 | 0 | 1 | 0 |
| African | (14.3) | (16.7) | (16.7) | (16.7) | | | (16.7) | |
| American | | | | | | | | |
| Asian | 0 | 0 | 0 | 0 | 0 | 1 | 1 | 1 |
| | | | | | | (16.7) | (16.7) | (16.7) |
| Other | 0 | 1 | 1 | 0 | 0 | 0 | 0 | 0 |
| | | (16.7) | (16.7) | | | | | |
| Weight, kg, | 73.9 | 69.9 | 67.8 | 68.8 | 64.5 | 74.7 | 75.5 | 71.2 |
| mean (SD) | (10.7) | (16.8) | (8.6) | (12.7) | (3.1) | (11.6 | (21.1) | (6.7) |

^{a}Pool of all administration types.
^{b}Multiple dose study drug administration given as single dose of 15 mg/kg IV + 400 mg SC once weekly for 4 weeks.
IV, intravenous; n, number of dosed subjects; SC, subcutaneous; SD, standard deviation.

### Pharmacokinetics and Pharmacodynamics

REGN3918 exhibited dose-dependent increases in exposure in serum, with a trend toward prolonged serum concentrations at IV doses ≥10 mg/kg (FIG. 2). Following SC administration, concentrations of REGN3918 in serum peaked at 4-8 days post-dose, and bioavailability was estimated as approximately 70%. REGN3918 exposure led to dose-dependent inhibition of CH50. In all 4 IV dosing cohorts, suppression of hemolysis was observed at 15 minutes post-injection. Maximal suppression of hemolysis was achieved with ≥3 mg/kg dosing. At 30 mg/kg, maximal suppression of hemolysis was maintained for ≥4 weeks, consistent with observed prolonged REGN3918 concentrations following this dose. In the 2 SC cohorts, peak suppression of hemolysis was observed 3-7 days post dosing, again consistent with observed peak concentrations of REGN3918 in serum. In the multiple dose Cohort 5, complete suppression of CH50 was observed over the 4-week dosing period and 2 weeks post the last dosing (FIG. 3).

### Safety

REGN3918 was found to be well tolerated in single doses of up to 30 mg/kg IV and 600 mg SC (Table 2-2). The multiple dose Cohort 5 has completed dosing in all subjects and has completed all safety follow-up. A single serious adverse event, salpingitis, occurred in 1 subject in Cohort 5; the serious adverse event occurred after completion of dosing and completely resolved after treatment with a short course of antibiotics.

TABLE 2-2
Overview of Treatment-Emergent Adverse Events (TEAEs)

| | | | | | | | | 15 mg/kg |
|---|---|---|---|---|---|---|---|---|
| n (%) of | Placebo^{a} | 1 mg/kg | 3 mg/kg | 10 mg/kg | 30 mg/kg | 300 mg | 600 mg | IV + 400 mg |
| subjects | (n = 14) | IV (n = 6) | IV (n = 6) | IV (n = 6) | IV (n = 6) | SC (n = 6) | SC (n = 6) | SC^{b} (n = 6) |
| Any TEAE | 13 | 5 | 4 | 5 | 6 | 4 | 6 | 3 |
| | (92.9) | (83.3) | (66.7) | (83.3) | (100) | (66.7) | (100) | (50.0) |
| Any serious | 0 | 0 | 0 | 0 | 0 | 0 | 0 | 1 |
| TEAE | | | | | | | | (16.7) |
| Any severe | 0 | 0 | 0 | 0 | 0 | 0 | 0 | 1 |
| TEAE | | | | | | | | (16.7) |
| Any TEAE | 0 | 0 | 0 | 0 | 0 | 0 | 0 | 0 |
| leading to study | | | | | | | | |
| withdrawal, | | | | | | | | |
| discontinuation | | | | | | | | |
| or death | | | | | | | | |

TEAEs occurring in ≥20% of subjects in any treatment group by preferred term^{c}

| | | | | | | | | |
|---|---|---|---|---|---|---|---|---|
| Diarrhea | 4 | 0 | 1 | 0 | 1 | 0 | 1 | 0 |
| | (28.6) | | (16.7) | | (16.7) | | (16.7) | |
| Dizziness | 3 | 1 | 1 | 1 | 0 | 1 | 0 | 0 |
| | (21.4) | (16.7) | (16.7) | (16.7) | | (16.7) | | |
| Nasopharyngitis | 3 | 1 | 2 | 2 | 2 | 3 | 1 | 0 |
| | (21.4) | (16.7) | (33.3) | (33.3) | (33.3) | (50.0) | (16.7) | |
| Nausea | 3 | 0 | 0 | 1 | 0 | 1 | 2 | 0 |
| | (21.4) | | | (16.7) | | (16.7) | (33.3) | |
| Vomiting | 3 | 0 | 0 | 2 | 1 | 1 | 1 | 0 |
| | (21.4) | | | (33.3) | (16.7) | (16.7) | (16.7) | |
| Headache | 2 | 0 | 1 | 1 | 1 | 1 | 2 | 1 |
| | (14.3) | | (16.7) | (16.7) | (16.7) | (16.7) | (33.3) | (16.7) |
| Oropharyngeal | 1 | 0 | 0 | 0 | 2 | 0 | 0 | 1 |
| pain | (7.1) | | | | (33.3) | | | (16.7) |
| Candida | 0 | 0 | 0 | 0 | 2 | 0 | 0 | 0 |
| infection | | | | | (33.3) | | | |
| Pollakiuria | 0 | 0 | 0 | 0 | 0 | 0 | 0 | 2 |
| | | | | | | | | (33.3) |

^{a}Pool of all administration types.
^{b}Multiple dose study drug administration given as single dose of 15 mg/kg IV + 400 mg SC once weekly for 4 weeks.
^{c}MedDRA (Version 21.0) coding dictionary applied.
IV, intravenous; SC, subcutaneous; TEAE, treatment-emergent adverse event.

REGN3918 was generally well tolerated in both single ascending IV and SC dose administration as well as in a single IV loading dose followed by 4 consecutive weekly dose administrations. Rapid and maximal suppression of complement activity as measured by the sheep red blood cell CH50 assay was demonstrated for IV doses with ≥3 mg/kg dosing. At 30 mg/kg, maximal suppression of hemolysis was maintained for ≥4 weeks. A regimen of 15 mg/kg IV loading dose followed by 4 consecutive weekly 400 mg SC doses maintained suppression of CH50 throughout the dosing period and 2 weeks post the last dosing.

### Hemolysis Assays

To further characterize the impact of REGN3918 on the alternative complement pathway (AP) activity, the effect of REGN3918 on alternative pathway-mediated hemolysis using an AH50 assay in the completed first-in-human (FIH) study was investigated. In addition, the effect of REGN3918 in both alternative and classical pathway hemolysis assays with those of eculizumab and ravulizumab in pooled normal human serum (NHS) samples, ex vivo, was compared.

Serum collected at multiple time-points was utilized to assess the effect of REGN3918 on alternative pathway activity. For ex vivo spike experiments, pooled NHS was used to compare the hemolytic function of REGN3918, eculizumab and ravulizumab. The alternative pathway (AP) and classical pathway (CP) hemolysis assays were performed based on lysis of rabbit red blood cells (RBCs) and sensitized sheep RBCs, respectively. Both assays measure the amount of hemoglobin released from red blood cells at 412 nm.

In the FIH study, baseline AH50 was comparable across treatment groups with a mean of 110 U/mL (standard deviation=19, n=56). REGN3918 exposure led to dose-dependent inhibition of AH50. In all 4 IV dosing cohorts, peak suppression of hemolysis was observed at end of infusion (EOI). Maximal suppression of hemolysis was approximately −85% change from baseline. This was achieved with the 30 mg/kg IV group and the repeat dose 15 mg/kg IV+400 mg SC QW group. In the 2 SC cohorts, peak suppression of hemolysis was observed 3-7 days post dosing, which was consistent with observed peak concentrations of REGN3918 in serum. In an ex vivo spike study, REGN3918, eculizumab and ravulizumab were spiked into 10, 25 or 48% pooled NHS for AP, and 5, 10 or 25% for CP. The results from AP hemolysis assays showed that, for a given concentration of spiked antibody, the maximal suppression of hemolysis for all the antibodies decreased with increased percentage of serum (FIG. 4(A-C); Table 2-3). The maximal suppression of hemolysis was consistently higher (32-169%) for REGN3918 relative to eculizumab, and lower for ravulizumab relative to REGN3918 and eculizumab at all serum percentages tested. The results from CP hemolysis assays showed that, although the maximal suppression of hemolysis was similar for all antibodies tested, ravulizumab was required to be at least a log higher in concentration to achieve a similar effect as the other two anti-C5 antibodies (FIG. 4 (D-F); Table 2-4).

Magnesium is an important cofactor for the activity of AP C3 and C5 convertases. By changing the serum percentage (10, 25 or 48%), magnesium concentration could change, which would affect the converatse function. To test if this is the underlying cause for the differences observed among the three antibodies tested at different serum percentages, we conducted the AP assay with 25% NHS and three different concentrations of magnesium. Magnesium concentration (MgCl₂) at 1, 1.5 or 2 mM did not affect the individual antibody performance. Also, the relative differences among three antibodies tested still existed at three different concentrations of magnesium. While magnesium concentration may still be a contributing factor, under the conditions tested, there seems to be other mechanisms that may be responsible for the relative differences observed.

Ex vivo studies with pooled NHS demonstrated that REGN3918 robustly blocked both CP and AP hemolysis. Ravulizumab appeared to be less potent compared with eculizumab in both CP and AP hemolysis assays. The Phase I healthy volunteer study of REGN3918 demonstrated dose-dependent and significant inhibition of alternative pathway hemolysis, with the maximal suppression of hemolysis approximately −85% change from baseline.

TABLE 2-3
Comparison of Maximal Suppression
of Hemolysis of REGN3918, Eculizumab
and Ravulizumab in AP Hemolysis Assay

| | Maximum % suppression | | |
|---|---|---|---|
| Antibody | 10% NHS | 25% NHS | 48% NHS |
| Pozelimab | 77.06 | 70.81 | 41.17 |
| (REGN3918) | | | |
| Eculizumab | 58.39 | 35.15 | 15.29 |
| Ravulizumab | 42.95 | 21.86 | 8.46 |
| Isotype control | N/A | N/A | N/A |
| (REGN1945*) | | | |

REGN1945 = Fel d 1 (Felis domesticus) antibody

TABLE 2-4
Comparison of Maximal Suppression
of Hemolysis of REGN3918, Eculizumab and
Ravulizumab in CP Hemolysis Assay

| | Maximum % suppression | | |
|---|---|---|---|
| Antibody | 5% NHS | 10% NHS | 25% NHS |
| Pozelimab (REGN3918) | 95.05 | 92.83 | 89.24 |
| Eculizumab | 92.23 | 87.15 | 89.31 |
| Ravulizumab | 84.41 | 78.84 | 80.45 |
| Isotype control (REGN1945) | N/A | N/A | N/A |

TABLE 2-5
Differences Among the Three Anti-05
Antibodies are Consistent across
Different Concentration of Mgcl₂ Tested

| | Maximum % suppression | | |
|---|---|---|---|
| Antibody | 1 mM MgC₂ | 1.5 mM MgC₂ | 2 mM MgC₂ |
| Pozelimab | 81.13 | 79.84 | 72.42 |
| (REGN3918) | | | |
| Eculizumab | 54.94 | 47.02 | 50.63 |
| Ravulizumab | 29.39 | 26.58 | 20.43 |
| Isotype control | N/A | N/A | N/A |
| (REGN1945) | | | |

The sequences of eculizumab and ravulizumab antibodies used in these assays were as follows:

Eculizumab
(SEQ ID NO: 358)
QVQLVQSGAEVKKPGASVKVSCKASGYIFSNYWIQ
WVRQAPGQGLEWMGEILPGSGSTEYTENFKDRVTM
TRDTSTSTVYMELSSLRSEDTAVYYCARYFFGSSP
NWYFDVWGQGTLVTVSSASTKGPSVFPLAPCSRST
SESTAALGCLVKDYFPEPVTVSWNSGALTSGVHTF
PAVLQSSGLYSLSSVVTVPSSNFGTQTYTCNVDHK
PSNTKVDKTVERKCCVECPPCPAPPVAGPSVFLFP
PKPKDTLMISRTPEVTCVVVDVSQEDPEVQFNWYV
DGVEVHNAKTKPREEQFNSTYRVVSVLTVLHQDWL
NGKEYKCKVSNKGLPSSIEKTISKAKGQPREPQVY
TLPPSQEEMTKNQVSLTCLVKGFYPSDIAVEWESN
GQPENNYKTTPPVLDSDGSFFLYSRLTVDKSRWQE
GNVFSCSVMHEALHNHYTQKSLSLSLGK
(SEQ ID NO: 359)
DIQMTQSPSSLSASVGDRVTITCGASENIYGALNW
YQQKPGKAPKLLIYGATNLADGVPSRFSGSGSGTD
FTLTISSLQPEDFATYYCQNVLNTPLTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
Ravulizumab
(SEQ ID NO: 360)
QVQLVQSGAEVKKPGASVKVSCKASGHIFSNYWIQ
WVRQAPGQGLEWMGEILPGSGHTEYTENFKDRVTM
TRDTSTSTVYMELSSLRSEDTAVYYCARYFFGSSP
NWYFDVWGQGTLVTVSSASTKGPSVFPLAPCSRST
SESTAALGCLVKDYFPEPVTVSWNSGALTSGVHTF
PAVLQSSGLYSLSSVVTVPSSNFGTQTYTCNVDHK
PSNTKVDKTVERKCCVECPPCPAPPVAGPSVFLFP
PKPKDTLMISRTPEVTCVVVDVSQEDPEVQFNWYV
DGVEVHNAKTKPREEQFNSTYRVVSVLTVLHQDWL
NGKEYKCKVSNKGLPSSIEKTISKAKGQPREPQVY
TLPPSQEEMTKNQVSLTCLVKGFYPSDIAVEWESN
GQPENNYKTTPPVLDSDGSFFLYSRLTVDKSRWQE
GNVFSCSVLHEALHSHYTQKSLSLSLGK
(SEQ ID NO: 361)
DIQMTQSPSSLSASVGDRVTITCGASENIYGALNW
YQQKPGKAPKLLIYGATNLADGVPSRFSGSGSGTD
FTLTISSLQPEDFATYYCQNVLNTPLTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC

### Example 3: Switching from Eculizumab to REGN3918 Results in Normalization of C5 Concentrations in C5^{hu/hu} Mice and Sustained Suppression of Hemolytic Activity Ex Vivo

To assess the effects of switching treatments from eculizumab to REGN3918, three groups of C5^{hu/hu} mice were administered three doses of 15 mg/kg of REGN3918 or eculizumab (SEQ ID NOs: 358 and 359) on days 0, 15 and 29. One group received REGN3918 only for all three doses, while a second group received eculizumab only. The third group, the ‘switch group’, received eculizumab on day 0 and was then switched to REGN3918 on days 15 and 29. Cage-side observations and routine wellness checks showed mice to be healthy with all animals surviving until their scheduled date of termination. Over the duration of the study, blood was sequentially collected at multiple points pre- and post-dosing. Cₘₐₓ values for REGN3918 and eculizumab were comparable following the first dose (151 and 144 μg/mL, respectively); however, REGN3918 alone demonstrated slower clearance (CL) compared to eculizumab alone, resulting in modestly higher serum concentrations for REGN3918 (FIG. 4A, Table 4). Following the switch from eculizumab to REGN3918, the concentration-versus time profile of total hIgG initially resembled the PK profile of REGN3918 during the second post-switch dosing interval (FIG. 26(A), Table 3-1). These results suggest that there is only a modest effect on the total IgG concentrations in serum upon switching from eculizumab to REGN3918 relative to the concentrations observed with either mAb administered as a single agent.

Serum C5 concentrations were also monitored. In mice administered REGN3918, serum concentrations of C5 increased to a maximum of 1.4-fold over the duration of the study. In contrast, eculizumab induced higher concentrations of serum C5 after the first, second and third doses (1.9, 2.0 and 2.8-fold increases, respectively) (FIG. 26(B)). The first dose of eculizumab administered to the ‘switch group’ induced a similar increase in serum C5 concentrations as in animals that received eculizumab alone. After switching treatment to REGN3918 on day 15, serum C5 concentrations in the ‘switch group’ transiently fell below baseline (70%), but returned to levels similar to the group administered REGN3918 alone after the final dose of REGN3918. The accelerated clearance of C5 following dose switching may be consistent with a transient formation of immune complexes containing REGN3918, eculizumab and C5, as demonstrated by A4F-MALLS studies. The effects of switching treatments from eculizumab to REGN3918 on the efficacy of blocking complement-mediated hemolysis were also assessed. Sera from terminally sacrificed C5^{hu/hu} mice (n=5) were collected prior to each new dose as well as 14 days post third dose and used in CP mediated hemolysis assays. Hemolysis was effectively blocked to a similar extent in serum collected from all three groups after the first, second and third administration of antibody (FIG. 26(C)). Blockage of hemolysis was associated with C5:mAb ratios that were maintained below one over the duration of the study (FIG. 26(D)). Collectively, these results indicate that switching treatments from eculizumab to REGN3918 was generally well tolerated and was associated with continued suppression of complement activation ex vivo.

TABLE 3-1
Pharmacokinetic Analysis of Mice Receiving REGN3918 or Eculizumab or the Switch Regimen.

| | | Day 0-15 | | | Day 16-29 | | | Day 30-43 | | |
|---|---|---|---|---|---|---|---|---|---|---|
| Parameter | Units | REGN3918 | Eculizumab | Switch | REGN3918 | Eculizumab | Switch | REGN3918 | Eculizumab | Switch |
| Cₘₐₓ | μg/mL | 151 ± | 144 ± | 162 ± | 253 ± | 200 ± | 238 ± | 298 ± | 203 ± | 291 ± |
| | | 18 | 15 | 10 | 12 | 27 | 4.2 | 22 | 20 | 14 |
| AUC_{Last} | d · μg /mL | 1800 ± | 1560 ± | 1770 ± | 2460 ± | 1750 ± | 1940 ± | 2960 ± | 2080 ± | 2860 ± |
| | | 230 | 310 | 160 | 240 | 380 | 71 | 450 | 74 | 540 |
| CL | mL/d/kg | 3.3 ± | 6.4 ± | 5.0 ± | 3.0 ± | 6.0 ± | 5.7 ± | 2.5 ± | 2.8 ± | 3.1 ± |
| | | 1 | 5 | 2 | 1 | 3 | 0.2 | 1 | 0.8 | 2 |
| CP Hemolysis | % | 23.4 ± | 33.2 ± | ND | 22.5 ± | 12.0 ± | 11.4 ± | 4.8 ± | 5.2 ± | 8.3 ± |
| | | 6.1 | 16.0 | | 8.0 | 8.4 | 0 | 0.23 | 2.3 | 2.2 |
| AP Hemolysis | % | 6.9 ± | 10.4 ± | ND | 8.2 ± | 6.8 ± | 4.7 ± | 5.6 ± | 9.3 ± | 7.2 ± |
| | | 0.4 | 5.1 | | 3.8 | 1.7 | 0 | 0.5 | 3.7 | 0.5 |

ND = Not Determined

REGN3918, eculizumab and C5 complexes predominantly contain 1 to 2 molecules of C5. REGN3918 has can be a viable therapeutic option for patients that carry rare genetic variants of C5 and may also provide an alternative for patients currently treated with eculizumab. However, combining antibodies that bind unique epitopes on a soluble antigen has the potential to generate higher order protein complexes, which can elicit type III hypersensitivity reactions similar to serum sickness. Such conditions are likely to be self-limiting and the size of the complexes will be influenced by the molar ratio of the antibodies and antigen, with the largest complexes generally forming when the components are at or near equimolar amounts. Here, we examined the size of complexes formed at 5:1:1 molar ratios of REGN3918: eculizumab:C5 by asymmetric flow field flow fractionation using multi-angle laser light scattering detection (A4F-MALLS). This molar ratio was chosen based on the serum concentrations that would be expected in vivo at the time of the initial dose switch in the clinic.

Representative fractograms generated following A4F-MALLS analysis of the eculizumab/C5/REGN3918 mixture and each of the individual components are overlaid in FIG. 27. A major peak (Peak 1; ˜66% total peak area, Table 3-2) representing free REGN3918 was detected in the simulated mixture, since the concentration of REGN3918 is likely in vast molar excess to both C5 and in-house eculizumab. Several additional minor peaks (Peaks 2-4) corresponding to heteromeric complexes of eculizumab, C5, and REGN3918 were also detected in this sample, confirming that both in-house eculizumab and REGN3918 can engage the same molecule of C5 to form extended antibody-antigen lattices. However, the majority of these complexes fractionate into two discrete peaks (Peaks 2 and 3; ˜22% total peak area) with calculated average molar masses of −499 kDa and ˜841 kDa. Based on the calculated molar masses of the individual components, peaks 2 and 3 likely represent 2:1 and 3:2 mAb:C5 heteromeric complexes, respectively. A broad, poorly-resolved peak (Peak 4) that likely corresponds to a heterogenous distribution of higher order complexes (˜1200-2100 kDa) was also detected, but only represented ˜12% of the total peak area. Taken together, these data suggest that, although eculizumab and REGN3918 can form heteromeric complexes with C5, the formation of very large, heterogeneous, and potentially immunogenic complexes is likely minimal when each component is present at concentrations expected in vivo at the time of the initial dose switch. Furthermore, the formation of these very large complexes is likely transient and should steadily decline as eculizumab is cleared from circulation and/or with additional doses of REGN3918.

TABLE 3-2
Size Distribution of Heteromeric Complexes Formed by REGN3918, Eculizumab and C5.

| | | | | | | Peak 2 | | Peak 3 | | Peak 4 | |
|---|---|---|---|---|---|---|---|---|---|---|---|
| | | | | Peak 1 | | [mAb]₂; [hC5]₁ | | [mAb]₃; [hC5]₂ | | Higher Order | |
| | | | | Free mAb/hC5 | | Complex | | Complex | | Heteromeric Complexes | |
| | | | | Mean | Mean | Mean | Mean | Mean | Mean | Mean | Mean |
| [REGN3918] | [eculizumab] | [hC5] | Approximate | MW | Peak | MW | Peak | MW | Peak | MW | Peak |
| μM | μM | μM | Molar Ratio | (kDa) | Area (%) | (kDa) | Area (%) | (kDa) | Area (%) | (kDa) | Area (%) |
| 6.7 | 0 | 0 | 1:0:0 | 153.9 | 100 | ND | ND | ND | ND | ND | ND |
| | | | | (0.9) | (0.0) | | | | | | |
| 0 | 6.7 | 0 | 0:1:0 | 151.6 | 100 | ND | ND | ND | ND | ND | ND |
| | | | | (0.0) | (0.0) | | | | | | |
| 0 | 0 | 5.3 | 0:0:1 | 191.8 | 100 | ND | ND | ND | ND | ND | ND |
| | | | | (0.5) | (0.0) | | | | | | |
| 4.3 | 0.7 | 0.8 | 5:1:1 | 153.5 | 64.6 | 498.8 | 12.6 | 840.9 | 9.1 | ~1200- | 13.8 |
| | | | | (0.5) | (0.6) | (3.7) | (0.7) | (3.2) | (0.6) | 2100 | (0.6) |

ND = Not Determined

In addition to providing a viable therapeutic option for patients carrying rare C5 variants, REGN3918 also offers an alternative to patients currently treated using eculizumab. For example, REGN3918 may require less frequent dosing regimens and result in more stable serum C5 levels. Dose switching studies in humanized C5 mice demonstrated that switching treatments from eculizumab to REGN3918 was well tolerated and maintained suppression of complement activity. However, combining antibody therapeutics against a soluble antigen has the potential to generate higher order, immunogenic protein complexes. Using molar ratios of eculizumab:REGN3918:hC5 that would be expected at the time of dose switching, A4F-MALS studies demonstrated eculizumab and REGN3918 can form heteromeric complexes with C5. However, the formation of very large, heterogeneous, and potentially immunogenic complexes was minimal and would likely be transient in vivo. These data may support using an excess of REGN3918 when dose switching from eculizumab to minimize the potential for inducing serum sickness-like reactions.

### Example 4: An Open-Label Efficacy and Safety Study of Pozelimab in Patients with CD55-Deficient Protein-Losing Enteropathy (CHAPLE Disease)

This is an open label, single arm, 104-week treatment study in patients aged 1 year and older with active clinical signs and symptoms of CD55-deficient PLE/CHAPLE disease, and a CD55 loss-of-function mutation detected by genotype analysis (frameshift, nonsense mutations). Patients will be given a single loading dose of pozelimab 30 mg/kg intravenously (IV) on day 1, then fixed doses subcutaneously (SC) (based on body weight) QW (±1 day) over the treatment period. The study includes a screening period (up to 4 weeks) followed by a 104-week treatment period from week 0 to week 103, and a follow-up period from week 104 to week 116. Only patients with active PLE will be included in the primary analysis. In this study, active PLE is defined as hypoalbuminemia of less than or equal to 3.2 g/dL within the screening period, and within the last 6 months, at least 7 days (which do not have to be consecutive) of 1 or more of the following symptoms or signs: diarrhea, vomiting, abdominal pain, peripheral or facial edema, an episode of infection with concomitant hypogammaglobulinemia, or a new thrombotic event.

### Study Duration

The duration of the study for a patient is approximately 117 weeks (from week 0 to week 116), excluding the screening period.

### Study Population

Sample Size. A minimum of 6 patients with active PLE will be enrolled. Following this, enrollment will be closed 1 year after the FPFD or upon enrollment of the 20th patient, whichever is earlier. Eligible patients with inactive PLE may also be enrolled, but their data will not be included in the primary analysis.

Target Population. Patients aged 1 year and older with a clinical diagnosis of CD55-deficient PLE disease, with CD55 loss of function mutation determined by genetic analysis (frameshift, nonsense mutations) and confirmed (only necessary in the case of missense or suspected splice site mutations) by flow cytometry or western blotting CD55 on peripheral blood cells. The first 2 patients must be aged 6 years and older (exception will be made for patients under 6 years of age with life-threatening disease).

TABLE 4-1
Demographics and Other Baseline Characteristics
of Four Patients in the Clinical Trial.

| | Age | | | | Weight | Height | BMI |
|---|---|---|---|---|---|---|---|
| Subject ID* | (Years) | Sex | Ethnicity | Race | (kg) | (cm) | (kg/m²) |
| 792001001 | 11 | M | Not Hispanic or | WHITE | 28 | 147 | 12.96 |
| | | | Latino | | | | |
| 792001004 | 5 | F | Not Hispanic or | WHITE | 11 | 95 | 12.19 |
| | | | Latino | | | | |
| 792001006 | 9 | F | Not Hispanic or | WHITE | 25 | 130 | 14.79 |
| | | | Latino | | | | |
| 792001007 | 8 | F | Not Hispanic or | WHITE | 22.2 | 110.5 | 18.18 |
| | | | Latino | | | | |

*Subjects are identified by anonymous numeric ID.

### Inclusion Criteria

A patient must meet the following criteria to be eligible for inclusion in the study:
1. Male or female aged 1 year and older. The first 2 patients recruited must be aged 6 years or older;
2. Clinical diagnosis of CD55-deficient PLE/CHAPLE disease (based on a history of PLE), confirmed by biallelic CD55 loss-of-function mutation detected by genotype analysis (frameshift, nonsense mutations). In the case of missense or suspected splice site mutations, CD55-deficient PLE is to have been confirmed by flow cytometry of peripheral blood cells or western blot. These diagnostic tests can be performed as part of the study screening procedures, or as part of standard clinical evaluation prior to screening;
3. Patient either has:
   a. Active disease, defined as:
   (i) Hypoalbuminemia of less than or equal to 3.2 g/dL within the screening period, and
   (ii) Within the last 6 months and attributable to CD55-deficient PLE, at least 7 days (which do not have to be consecutive) of at least one of the following symptoms or signs: diarrhea, vomiting, abdominal pain, peripheral or facial edema, or an episode of infection with concomitant hypogammaglobulinemia, or a new thromboembolic event.
   NOTE: The first 2 patients enrolled in the study must fall into inclusion criterion 3a.
   b. Inactive disease on eculizumab therapy (and whose treating physician has the expectation of future access to renewed eculizumab treatment should this be required), and is willing to discontinue eculizumab during screening and start pozelimab at baseline with no eculizumab wash-out;
4. Willing and able to comply with clinic visits and study-related procedures;
5. Written informed consent from parent/guardian for minor patients;
6. Written assent from minor patients as appropriate (e.g., above the age of 6 years or the applicable age per local regulatory requirements); and
7. Patient either alone or with the help of their parents/legal guardians, as required, must be able to understand and complete study-related questionnaires.

### Exclusion Criteria

A patient who meets any of the following criteria will be excluded from the study:

1. History of meningococcal infection.
2. No documented meningococcal vaccination within 3 years prior to screening and patient unwilling to undergo vaccination during the study (if fully available according to local practice).
3. No documented vaccination for Haemophilus influenzae and Streptococcus pneumoniae if applicable based on local practice or guidelines prior to screening and patient unwilling to undergo vaccination during the study if required per local practice or guidelines.
4. Presence of a concomitant disease that leads to hypoproteinemia at the time of starting pozelimab, including a urinary protein loss or a hepatic disease that affects production of proteins by liver.
5. A concomitant disease that leads to secondary intestinal lymphangiectasia such as a fontan procedure for congenital heart disease.
6. Recent infection requiring systemic treatment with antibiotics, antivirals, or antifungals (within 2 weeks of screening or during the screening period). If the patient is appropriately treated, the patient may be rescreened.
7. PLE previously refractory to eculizumab, with the exception of patients with the Arg885His variant in the C5 gene.
8. Known hereditary complement deficiency other than CD55 deficiency.
9. Documented history of active, ongoing systemic autoimmune diseases.
10. Known or suspected infectious colitis at screening. Once this has resolved, patient may be rescreened.
11. Patients with an estimated glomerular filtration rate (eGFR) of <30 mL/min/1.73 m² (according to Chronic Kidney Disease—Epidemiology Collaboration equation 2009 [adults] or creatinine-based Schwartz equation [pediatric patients]).
12. Recent, unstable medical conditions, excluding PLE and related complications, within the past 3 months prior to screening visit. Option to rescreen after 3 months has elapsed.
13. Known sensitivity to any of the components of the pozelimab formulation or drug product.
14. Any clinically significant abnormality identified at the time of screening that, in the judgment of the investigator or any sub-investigator, would preclude safe completion of the study or constrain endpoints assessment, such as major systemic diseases, including a medical history of hepatitis B or C. Patients known to have had hepatitis B or C in the past can enroll only if these diseases are no longer active, as demonstrated by negative hepatitis B surface antigen (HBsAg), hepatitis B e antigen (HBeAg), hepatitis B virus DNA, and negative hepatitis C virus RNA (HCV RNA), respectively.
Note: Testing for hepatitis B and C is not mandatory for enrollment in the trial but may be performed at the discretion of the investigator.
15. Participation in another interventional clinical study or use of any experimental therapy within 30 days before screening visit or within 5 half-lives of that investigational product, whichever is greater, with the exception of complement inhibitors.
16. Considered by the investigator or any sub-investigator as inappropriate for this study for any reason, e.g.:
Deemed unable to meet specific protocol requirements, such as scheduled visits and/or
Deemed unable to tolerate long-term injections as per the patient, the investigator, sub-investigator, pharmacist, study coordinator, other study staff, or relative thereof directly involved in the conduct of the study, etc. and/or
Presence of any other conditions (e.g., geographic, social, etc.), actual or anticipated, that the investigator feels would restrict or limit the patient's participation for the duration of the study
   17. Patients who are committed to an institution by virtue of an order issued either by the judicial or the administrative authorities.
   18. Women who are pregnant, breastfeeding, or who have a positive pregnancy test at screening visit or day 1.
   19. Pregnant or breastfeeding women.
   20. Women of childbearing potential* and girls beyond menarche (and not sexually abstinent) who are unwilling to practice highly effective contraception prior to the initial dose/start of the first treatment, during the study, and for at least 21 weeks after the last dose. Highly effective contraceptive measures include:
a. Stable use of combined (estrogen and progestogen-containing) hormonal contraception (oral, intravaginal, transdermal) or progestogen-only hormonal contraception (oral, injectable, implantable) associated with inhibition of ovulation initiated 2 or more menstrual cycles prior to screening
b. Intrauterine device (IUD); intrauterine hormone-releasing system (IUS)
c. Bilateral tubal ligation
d. Vasectomized partner
e. And/or sexual abstinence†, ‡. * Postmenopausal women must be amenorrheic for at least 12 months in order not to be considered of childbearing potential. Pregnancy testing and contraception are not required for women with documented hysterectomy or tubal ligation.† Sexual abstinence is considered a highly effective method only if defined as refraining from heterosexual intercourse during the entire period of risk associated with the study treatments. The reliability of sexual abstinence needs to be evaluated in relation to the duration of the clinical trial and the preferred and usual lifestyle of the patient.‡ Periodic abstinence (calendar, symptothermal, post-ovulation methods), withdrawal (coitus interruptus), spermicides only, and lactational amenorrhoea method (LAM) are not acceptable methods of contraception. Female condom and male condom should not be used together.
   21. Intentionally left blank
   22. Documented history of unresolved tuberculosis (TB), or evidence of active or latent tuberculosis infection (LTBI) during screening period. Assessment for active TB and LTBI should accord with local practice or guidelines, including those pertaining to risk assessment, and the use of tuberculin skin test or T-cell interferon-gamma release assay.

### Outcomes/Endpoints

The primary endpoint is the proportion of patients achieving both of the following:
Normalization of serum albumin, defined as
   serum albumin within the normal range at least 70% of measurements between week 12 and week 24, and
   no single albumin measurement of <2.5 g/dL between week 12 and week 24, and
   no requirement for albumin infusion between week 12 and week 24
Improvement in the following clinical outcomes that were evaluable for improvement at baseline, with no worsening of the others (i.e., those not evaluable for improvement) at week 24:
   The number of bowel movements per day, based on a 1-week average, captured by e-diary. Improvement is defined as a reduction of 50% or more in the number of daily bowel movements based on a 1-week average. Patients evaluable for improvement are defined as those with an average of 3 or more bowel movements per day at baseline. Worsening is defined as an increase of 30% or more.
   Physician assessment of facial edema (based on a 5-point Likert scale). Improvement is defined as a reduction of 2 points or more. Patients evaluable for improvement are defined as those with a severity of at least 2 points out of 5 at baseline. Worsening is defined as an increase of 2 points or more.
   Physician assessment of peripheral edema (based on a 5-point Likert scale). Improvement is defined as a reduction of 2 points or more. Patients evaluable for improvement are defined as those with a severity of at least 2 points out of 5 at baseline. Worsening is defined as an increase of 2 points or more.
   Patient/caregiver assessment of abdominal pain frequency as assessed by the Stomach pain and hurt sub-scale of the PedsQL™ GI Symptom Scale. Improvement is defined as an increase of 6 points or more (on the 0 to 100 transformed total subscale score where lower scores indicate worse GI stomach pain and hurt). Patients evaluable for improvement are defined as those with a score of 70 points or less at baseline. Worsening is defined as a decrease of 6 points or more.

The secondary endpoints are:
Incidence and severity of treatment-emergent adverse events (TEAEs) and other safety variables from baseline to week 104
Improvement in each patient's most bothersome sign/symptom at week 24, as determined prior to baseline using a semi-structured concept elicitation interview, from amongst the ‘core’ clinical endpoints of frequency of bowel movements, peripheral edema, facial edema, abdominal pain frequency, nausea, vomiting, and stool consistency:
   Improvement in nausea and vomiting will be defined as an increase of 6 points on the 0 to 100 transformed nausea and vomiting subscale of the PedsQL GI Symptom Scale score where lower scores indicate worse nausea and vomiting. Patients will be evaluable for improvement in nausea and vomiting if they have a score 585 on the nausea and vomiting subscale at baseline
   Improvement in stool consistency will be defined as a reduction of 50% in the number of days per week that the patient has a bowel movement of loose/watery consistency. A bowel movement is considered to be loose/watery if it corresponds to 3 images of loose or watery stools on the Brussels Infant and Toddler Stool Scale (BITSS), the images and descriptors for categories 4 or 5 on the modified Bristol Stool Form Scale for Children (mBSFS-C), and the images and descriptors for categories 6 or 7 of the Bristol Stool Form Scale (BSFS). To be evaluable for improvement in stool consistency, patients must have a bowel movement of loose/watery stool consistency for ≥2 days/week at baseline
The proportion of patients with active disease at baseline who maintain disease control at week 48 and week 104 as defined by:
   Normalization of serum albumin, defined as serum albumin within the normal range at at least 70% of measurements between week 12 and week 48 (and between week 12 and week 104); and no single albumin measurement of <2.5 g/dL between week 12 and week 48 (and week 104); and no requirement for albumin infusion between week 12 and week 48 (and week 104), and
   No worsening of facial or peripheral edema, increase in bowel movement, or increase in abdominal pain frequency between week 12 and week 48 (and week 104) using definitions of worsening as in the primary endpoint
   No increase in dose of permitted concomitant medication for the treatment of PLE at any time, no re-introduction of any permitted concomitant medication once withdrawn, where permitted concomitant medication is as follows: corticosteroids, IV or SC immunoglobulin, IV albumin, biologic immunomodulators (anti-TNF, vedolizumab), small molecule immunomodulators (e.g., azathioprine, mesalazine), micronutrients, enteral or parenteral Supplementation
The proportion of patients with inactive disease on eculizumab at baseline who maintain disease control at week 24, week 48, and week 104 as defined by:
   Normalization of serum albumin, defined as serum albumin within the normal range at at least 70% of measurements between week 12 and week 24 (and between week 12 and week 48, between week 12 and week 104); and no single albumin measurement of <2.5 g/dL between week 12 and week 24 (and week 48, week 104); and no requirement for albumin infusion between week 12 and week 24 (and week 48, week 104); and
   No worsening of facial or peripheral edema, increase in bowel movement, or increase in abdominal pain frequency between week 12 and week 24 (and week 48, week 104) using definitions of worsening as in the primary endpoint
   No increase in dose of permitted concomitant medication for the treatment of PLE at any time, no re-introduction of any permitted concomitant medication once withdrawn, where permitted concomitant medication is as follows: corticosteroids, IV or SC immunoglobulin, IV albumin, biologic immunomodulators (anti-TNF, vedolizumab), small molecule immunomodulators (e.g., azathioprine, mesalazine), micronutrients, enteral or parenteral supplementation
The number of bowel movements per day, based on a 1-week average, captured by e-diary from baseline to week 24
The number of days/week with ≥1 bowel movement of loose/watery stool consistency, as measured by BSFS for patients who are 18 years of age and older, mBSFS-C for patients who are toilet-trained and less than 18 years of age, or the BITSS who are not toilet-trained, and captured by e-diary from baseline to week 24
Physician assessment of facial edema (based on a 5-point Likert scale) from baseline to week 104
Physician assessment of peripheral edema (based on a 5-point Likert scale) from baseline to week 104
Change in abdominal symptoms, as assessed by the PedsQL™ GI Symptom Scale stomach pain and hurt sub-scale and food and drink limits sub-scale from baseline to week 104
Health-related quality of life as assessed by the PedsQL™ Generic Core Scales from baseline to week 104; additionally, the following sub-scales will be reported separately:
   About my work/studies and school functioning sub-scale
   Physical functioning sub-scale
Assessment of abdominal ascites (assessed by measurement of abdominal circumference) from baseline to week 24
Frequency of albumin infusions up to week 104, expressed as number per half-year. Albumin infusions are permitted during the treatment phase in the event that the albumin level is below 3.0 g/dL at 2 consecutive visits with accompanying symptoms of facial or peripheral edema or ascites. Any albumin infusions between week 12 and week 24 will render the patient a non-responder for the primary endpoint.
Total albumin, protein, total Ig, IgG, IgM, IgA, expressed as:
   Absolute value at every scheduled time point including week 24
   Absolute and percent change from baseline over time
   Time to first normalization
Vitamin B12, folate, iron, iron binding capacity, ferritin, magnesium, fasting cholesterol/triglycerides, expressed as:
   Absolute value at every scheduled time point including week 24
   Change from baseline over time
   Time to first normalization
Alpha-1 antitrypsin levels in blood and stool, and change from baseline to week 12 and week 24
Use and dose/frequency from baseline to week 104 of: corticosteroids, IV or SC immunoglobulin, IV albumin, biologic immunomodulators (anti-TNF, vedolizumab), small molecule immunomodulators (e.g., azathioprine, mesalazine), micronutrients, enteral or parenteral supplementation, anti-coagulants (e.g., low-molecular-weight heparin), antibiotics (with the exception of those used for the purpose of Neisserial prophylaxis), anti-platelet agents (e.g., low-dose aspirin)
Hospitalization days (percentage of days hospitalized) over time
Body weight and height (expressed as z scores) over time
Concentrations of total pozelimab in serum assessed throughout the study
Incidence of treatment-emergent anti-drug antibodies (ADA) to pozelimab in patients over time
Change and percent change from baseline of total complement activity CH50 over time

The exploratory outcomes are:
Total C5 concentrations in plasma over time
Markers of thrombosis: D-dimer, and N terminal prothrombin fragments (F1+2)
Complement assays: sC5b-9
Change in GI symptoms (Diarrhea sub-scale and Nausea and vomiting sub-scale) as measured by the Pediatric Quality of Life Inventory (PedsQL™) GI Symptoms Scales from baseline over time
Change in caregiver well-being and burden as measured by the PedsQL™ Family Impact Module from baseline over time
Clinician global impression of change (CGIC) from baseline to week 104
Clinician global impression of severity (CGIS) from baseline to week 104
Patient/caregiver global impression of change (PGIC/CareGIC) from baseline to week 104
Patient/caregiver global impression of severity (PGIS/CareGIS) from baseline to week 104
If appropriate to age and stage of sexual maturation, the Tanner pubertal stage
Whole exome sequencing (if not already done)

### Efficacy Measures/Procedures

Serum Albumin, Total Protein, and Immunoglobulin. Samples will be collected and tested in the blood chemistry or immunoglobin panel at a lab.

Physician Assessment of Edema and Ascites. Physicians will assess peripheral edema as follows: Following a general inspection and palpation of all 4 limbs, the investigator will rate the overall severity of peripheral edema taking into account both degree and distribution, on a 5-point rating scale, where 1 signifies no edema and 5 signifies very severe edema.

Physicians will assess facial edema as follows: Following a general inspection of the face, the investigator will rate the overall severity of facial edema, taking into account both degree and distribution, on a 5-point rating scale, where 1 signifies no edema and 5 signifies very severe edema.

Ascites severity will be assessed by measurement of abdominal circumference, as follows:
1. Palpate for the lower rib margin (costal margin) and mark with a short horizontal line;
2. Palpate for the iliac crest and mark with a short horizontal line;
3. Using the tape measure, measure the mid-distance between the two horizontal lines and mark this with another short horizontal line in the middle;
4. Ask the patient to cross their arms across their chest so that you have access to the waist. Instruct them to stand relaxed and look straight ahead. Try to make sure that they don't deliberately hold themselves in or out;
5. Pass the tape around the waist, making sure it is level and that it is positioned at the mid-distance mark on both sides;
6. Make sure the tape is not pulled too tight. It should rest on the skin but not indent it.
7. Make the measurement at the end of expiration;
8. Measure to the nearest 0.1 cm (1 mm);
9. Make 3 measurements of waist circumference; and
10. Record all three measurements and the mean (average) by adding the values together and dividing by 3.

In case of abnormal findings, these assessments should be accompanied by clinical photography as available. All physician assessments for a patient should be performed by the same investigator until after week 24.

### Study Design

This is an open-label, single-arm, 104-week treatment study in patients aged 1 year and older with active clinical signs and symptoms of CD55-deficient PLE/CHAPLE disease, and a CD55 loss-of-function mutation detected by genotype analysis (frameshift, nonsense mutations). In the case of missense or suspected splice site mutations, CD55-deficient PLE is to be confirmed by flow cytometry of peripheral blood cells. The first 2 patients enrolled will be of age 6 or older (exception will be made for patients under 6 with life threatening disease).

A minimum of 6 patients with active PLE will be enrolled. Following this, enrollment will be closed 1 year after the first patient first dose (FPFD) or upon enrollment of the 20th patient, whichever is sooner. The primary analysis will occur when approximately 6 patients with active PLE have received 6 months of treatment. Subsequent analyses will occur 1 and 2 years after the first dose in the last patient enrolled.

Patients will be given a single loading dose of pozelimab 30 mg/kg IV on day 1, then fixed doses SC (based on body weight) QW (±1 day) over the treatment period.

The study consists of a screening period (up to 4 weeks) followed by a 104-week treatment period from week 0 to week 103, and a follow-up period from week 104 to week 116. Following the end of the treatment period, patients may have the option to enroll in an open-label extension study, to last until approval of commercialization of pozelimab in their country if this has not already occurred, or until termination of commercialization/development of pozelimab.

Active PLE is defined as hypoalbuminemia of less than or equal to 3.2 g/dL within the screening period, and within the last 6 months, at least 7 days (which do not have to be consecutive) of one or more of the following symptoms or signs: diarrhea, vomiting, abdominal pain, peripheral or facial edema, or an episode of infection with concomitant hypogammaglobulinemia, or a new thrombotic event. Active patients should not be on current therapy with eculizumab.

### Investigational Drug

Pozelimab drug product will be provided in a sterile, single-use glass vial for either IV or SC administration and will be supplied by the sponsor. Drug product will be initially provided in lyophilized form in a sterile, single use glass vial for IV or SC administration that requires reconstitution with sterile water for injection, and then transitioned to a sterile, single-use glass vial or pre-filled syringe containing a liquid 200 mg/mL pozelimab formulation for IV or SC administration that will not require reconstitution.

Study drug will be supplied by the sponsor. The admixture solutions needed for delivery of the lyophilized or liquid drug product for IV administration will be sourced locally, or may be supplied by the sponsor, as necessary.

### Dosage and Administration

Patients will be given a single loading dose of pozelimab—30 mg/kg IV on day 1—then SC dosing QW (±2 days) over the treatment period based on body weight. The last dose of study drug is administered at week 103.

Subcutaneous dose regimen:
For BW <10 kg: 125 mg;
For BW ≥10 kg and <20 kg: 200 mg;
For BW ≥20 kg and <40 kg: 350 mg;
For BW ≥40 kg and <60 kg: 500 mg;
For BW ≥60 kg: 800 mg.

The location and administration options for the SC route of administration will depend on the preference of the investigator and patient (e.g., abdomen, thigh, or upper arm), the availability of clinical supply, and home healthcare visiting professional. Clinic visits for SC administration may or may not be needed.

If self-administration/administration by patient/designated person is allowed locally, then sufficient injection training at the scheduled injection with pozelimab will be provided. After training, observation of self-administration/administration by patient/designated person will be conducted by clinical site personnel or visiting healthcare professional. Once this observation is considered satisfactory, then the study drug can be subsequently administered independently by patient/designated person for the remainder of the study.

In addition, a patient diary will be provided prior to initiation of self-administration (i.e., day 29). The diary should be completed upon each study drug administration. A study drug kit will be dispensed at the clinical site visit, using a direct-to-patient (DTP) service provider, or transported by a healthcare professional, as applicable. Detailed information about the study drug administration is provided in the pharmacy manual.

### Pharmacokinetics (PK)

Analysis of Drug Concentration Data. The PK endpoint is concentration of total pozelimab in serum over time.

Summary of total drug concentrations and total C5 will be presented by nominal time point (i.e., the time points specified in the protocol). Individual data will be presented by actual time. Plots of the concentrations of pozelimab and total C5 will be presented over time (linear and log scales). When the scale is linear, concentrations below the lower limit of quantification (LLOQ) will be set to zero. In the log-scaled figures, concentrations below the LLOQ will be imputed as LLOQ/2. Summary statistics of concentrations of total pozelimab and total C5 may include, but are not limited to, arithmetic mean, standard deviation, standard error of the mean, coefficient of variation (in %), minimum, Q1, median, Q3, and maximum. No formal statistical analysis will be performed.

Analysis of Anti-Drug Antibody Data. Anti-drug antibodies will be characterized by the type and level of the observed response. Samples positive in the ADA assay will be further characterized for neutralizing antibodies (NAbs) and ADA titers.

Anti-drug antibodies response categories and titer categories that will be assessed are as follows:
Negative/preexisting immunoreactivity;
Treatment-emergent response;
Treatment-boosted response;
NAb response in ADA-positive patients;
Titer value category (titer range);
   Low (titer <1,000),
   Moderate (1,000≤titer ≤10,000),
   High (tier >10,000).

Listings of ADA assay results, treatment-emergent ADAs, NAbs, and titers presented by patient, time point, and dose cohort/group will be provided. Incidence of treatment-emergent ADAs and NAbs will be assessed as absolute occurrence (N) and percent of patients (%), grouped by ADA titer level.

Plots of drug concentrations will be examined and the influence of ADAs on individual PK profiles evaluated. Assessment of impact of ADAs on safety and efficacy may be provided.

### Pre-Treatments

Enrolled patients will require evidence of meningococcal immunization or administration of vaccination during the screening period, and oral antibiotics are recommended during the treatment period, according to local or national practice and investigator's assessment.

Vaccinations. Enrolled patients will require immunization with meningococcal vaccinations. Administration of vaccination should occur preferably at least 2 weeks prior to initiation of pozelimab, or at another time point according to local practice or national guidelines. It is suggested that patients undergo vaccination for serotypes A, C, Y, W, and, if available, serotype B. Patients who have had previous, documented vaccination for meningococcus will be re-immunized based on local practice. Patients should be closely monitored for early signs and symptoms of meningococcal infection and evaluated immediately if an infection is suspected. Patients will be provided with a patient safety card describing signs and symptoms of meningococcal infection along with instructions in case of a potential meningococcal infection, as well as information for the non-investigator healthcare provider.

It is recommended that pediatric patients have evidence of Haemophilus influenzae and Streptococcus pneumoniae immunizations, or administration of vaccinations during the screening period or during the treatment period, according to local practice, guidelines, and availability. The vaccinations will be sourced locally by the investigator or designee and reimbursed by the sponsor.

Oral Antibiotics. Daily, oral antibiotic prophylaxis may commence on the day of first dosing, unless the risks outweigh the benefits or it is inconsistent with local practice, and continue for the duration of the study. It is recommended that patients who prematurely discontinue pozelimab receive at least 21 weeks of oral antibiotic prophylaxis after discontinuing pozelimab, or a duration consistent with local guidelines, whichever is longer. For adults, it is suggested that antibiotic prophylaxis be penicillin V 500 mg twice a day (BID), and in the case of penicillin allergy, erythromycin 500 mg BID may be used at the discretion of the investigator. For pediatric patients, it is suggested that antibiotic prophylaxis be penicillin VK 125 mg orally BID in patients who are <5 years of age, and 250 mg BID if ≥5 years of age. If pediatric patients are penicillin-allergic, then erythromycin 125 mg orally BID in patients who are <3 years of age and 250 mg orally BID in patients who are ≥3 years of age. Ultimately, the decision to administer prophylaxis with oral antibiotics, the duration of prophylaxis, the choice and dosage regimen of oral antibiotics will be at the discretion of the investigator. The oral antibiotics will be sourced locally by the investigator or designee and reimbursed by the sponsor.

### Dose Modification and Study Treatment Discontinuation Rules

Dose Modification. Dose regimen modification/reduction is not allowed for an individual patient. Patients will increase dose as specified by the dose regimen in the event that they move into a higher BW bracket. For the purposes of these dose increases, body weight will be measured at the study visits as specified in the schedule of assessments and not at each weekly administration. Pozelimab will be supplied initially in vials as lyophilized powder for reconstitution, so a single presentation will support all the weight-based dosing regimen. The correct number of vials and volume for SC injection drawn up will be administered by a healthcare practitioner (not necessarily a doctor) at the study site, during a visit, or at a local primary healthcare clinic in between visits or at home; self-administration/administration by patient/designated person may also be allowed. Each SC dose may be administered by more than one injection if necessary; each injection should not exceed a 2 mL volume.

Study Drug Discontinuation. Patients who permanently discontinue from study drug and who do not withdraw from the study will be asked to return to the clinic for all remaining study visits per the visit schedule. Patients who permanently discontinue from study drug and who opt to withdraw from the study may be asked to complete study assessments.

Reasons for Permanent Discontinuation of Study Drug. Study drug dosing will be permanently stopped in the event of:
Serious or severe allergic reactions considered related to study drug;
Liver impairment as evidenced by one or more of the following criteria occurring without evidence of another etiology:
   Alanine aminotransferase (ALT) or aspartate aminotransferase (AST) >8× ULN, or
   ALT or AST >5×ULN for more than 2 weeks, or
   ALT or AST >3×ULN and total bilirubin >2×ULN (or international normalized ratio [INR]>1.5) and no other reason can be found to explain the combination of increased AST/ALT and total bilirubin, such as viral hepatitis A, B, or C; preexisting or acute liver disease; or another drug capable of causing the observed injury;
Patient withdraws consent;
Patient noncompliance (e.g., not complying with protocol-required visits, assessments, and/or dosing instructions); or
Investigator's clinical judgment that it is in the best interest of the patient.
   Note: Evidence of pregnancy is not considered an automatic reason for permanent discontinuation and should be discussed with the medical monitor. Pregnancy may be a reason for permanent discontinuation if the benefit-risk assessment of continuing treatment with pozelimab is deemed unfavorable.

Reasons for Temporary Discontinuation of Study Drug. Temporary discontinuation may be considered by the investigator because of suspected AEs. The investigator can reinitiate treatment with study drug under close and appropriate clinical and/or laboratory monitoring once the investigator will have considered, according to his/her best medical judgment, that the responsibility of the study drug in the occurrence of the concerned event was unlikely.

### Management of Acute Reactions

Acute Intravenous Infusion Reactions. Patients should be observed for 30 minutes after the infusion. Emergency equipment and medication for the treatment of infusion reactions must be available for immediate use. All infusion reactions must be reported as AEs and graded using the grading scales.

Interruption of the Intravenous Infusion. The infusion should be interrupted if any of the following AEs are observed:
Cough;
Rigors/chills;
Rash, pruritus (itching);
Urticaria (hives, welts, wheals);
Diaphoresis (sweating);
Hypotension;
Dyspnea (shortness of breath);
Vomiting; or
Flushing.

The reaction(s) should be treated symptomatically, and the infusion may be restarted at 50% of the original rate.

If investigators feel there is a medical need for treatment or discontinuation of the infusion other than described above, they should use clinical judgment to provide the appropriate response according to typical clinical practice.

Termination of the Intravenous Infusion. The infusion should be terminated and NOT restarted if any of the following AEs occur:
Anaphylaxis*;
Laryngeal/pharyngeal edema;
Severe bronchospasm;
Chest pain;
Seizure;
Severe hypotension;
Other neurological symptoms (confusion, loss of consciousness, paresthesia, paralysis, etc.); or
Any other symptom or sign that, in the opinion of the investigator, warrants termination of the IV infusion *Consider anaphylaxis if the following is observed (Sampson et al., Second symposium on the definition and management of anaphylaxis: summary report—second National Institute of Allergy and Infectious Disease/Food Allergy and Anaphylaxis Network Symposium. Ann Emerg Med 2006; 47(4):373-80): acute onset of an illness (minutes to several hours) with involvement of the skin, mucosal tissue, or both (e.g., generalized hives, pruritus or flushing, swollen lips-tongue-uvula) and at least one of the following:
Respiratory compromise (e.g., dyspnea, wheeze-bronchospasm, stridor, reduced peak expiratory flow, hypoxemia); or
Reduced BP or associated symptoms of end-organ dysfunction (e.g., hypotonia [collapse], syncope, incontinence)

Systemic Injection Reactions. Patients should be observed for 30 minutes after the first SC injection. Emergency equipment and medication for the treatment of systemic reactions must be available for immediate use. All injection reactions must be reported as AEs and graded using the grading scales. Acute systemic reactions following SC injection of study drug should be treated using clinical judgment to determine the appropriate response according to typical clinical practice.

Local Injection Site Reactions. Local injection site reactions must be reported as AEs and graded according to the Food and Drug Administration (FDA) September 2007 Guidance for Industry, Toxicity Grading Scale for Healthy Adult and Adolescent Volunteers Enrolled in Preventive Vaccine Clinical Trials.

### Concomitant Medications

Any treatment administered from the time of informed consent to the end of the final study visit will be considered concomitant medication. This includes medications that were started before the study and are ongoing during the study.

Prohibited Medications. The following medications are prohibited, with the exception of those which are permitted as discussed below:
Within 24 hours prior to each clinic visit when blood is drawn, patients should not consume any alcohol;
Beginning on day 1 and continuing throughout the study, while the patient is continuing pozelimab, the patient should not take eculizumab;
Add any experimental therapy, including complement inhibitors even if they become approved during study conduct;
No Vitamin B12 supplementation during the first 4 weeks of pozelimab treatment (i.e., cannot be initiated prior to week 4 visit).

Permitted Medications. Permitted medication is any medication that is not prohibited. The following medications and procedures will be permitted, under the following conditions:
Albumin infusions are permitted during screening only for disease of life-threatening severity and after start of study drug in the event that the albumin level is below 3.0 g/dL with accompanying symptoms of facial or peripheral edema or ascites. This limitation only applies to albumin infusions given specifically for the PLE.
Any medication required to treat an AE, including non-steroidal anti-inflammatory drugs, antihistamines, or topical or systemic corticosteroids, at the discretion of the investigator;
Meningococcal vaccination;
Oral antibiotic prophylaxis;
Medications for treatment of type III hypersensitivity reactions;
Oral contraceptives or hormone replacement therapy may continue or be started during the study;
Acetaminophen/paracetamol, aspirin, or ibuprofen at the recommended dose per the local label;
Immunosuppressive drugs, biologic therapies, immunoglobulins, corticosteroids, anti-thrombotic agents, anticoagulants, antibiotics, iron supplements, vitamins, and enteral and parenteral feeding are permitted. Any changes to these concomitant medications will be at the discretion of the investigator. Weaning and/or withdrawal of any of these medications is permitted at the discretion of the investigator, in the context of response in the underlying disease to treatment with pozelimab; or
Any medication required for the treatment of patient's background medical conditions.

TABLE 4-1
Schedule of Events (Visits 1-11)

| Study procedure | | | | | | | | | | |
|---|---|---|---|---|---|---|---|---|---|---|
| Screening²⁹ | Baseline | Treatment period¹ | | | | | | | | |
| Visit No | | | | | | | | | | |
| 1 | 2 | 3 | 4 | 5 | 6 | 7 | 8 | 9 | 10 | 11 |
| Day | | | | | | | | | | |
| | | | 8 ± | 15 ± | 22 ± | 29 ± | 43 ± | 57 ± | 71 ± | 85 ± |
| −28 | 1 | 2 | 3 | 3 | 3 | 3 | 3 | 3 | 3 | 3 |
| Week | | | | | | | | | | |
| −4 | 0 | | 7 | 2 | 3 | 4 | 6 | 8 | 10 | 12 |

Screening/Baseline

| | | |
|---|---|---|
| Informed Consent | X | |
| Inclusion/Exclusion² | X | X |
| Genetic testing | X | |
| (if needed)² | | |
| Medical History³ | X | |
| Demographics | X | |
| Prior Medications⁴ | X | |
| Lab Parameter | X | |
| History⁵ | | |
| Vaccination History⁶ | X | X |
| Risk assessment | X | |
| for Neisseria | | |
| (as applicable)⁷ | | |
| Gonorrhea | X | |
| TB history and | X | |
| assessment⁸ | | |
| Electrocardiogram | X | |
| Concept Elicitation | X | |
| Interview⁹ | | |

Treatment

| Pozelimab Admin¹⁰ | | X¹¹ | | X¹² | | | | | | | |
|---|---|---|---|---|---|---|---|---|---|---|---|
| Patient Diary | | | | | | | X | X | X | X | X |
| (for self-admin) | | | | | | | | | | | |
| Concomitant Meds | X | X | X | X | X | X | X | X | X | X | X |
| and Interventions | | | | | | | | | | | |

Efficacy

| | | | | | | | | | | | |
|---|---|---|---|---|---|---|---|---|---|---|---|
| Tanner Staging¹³ | | X | | | | | | | | | X |
| e-Diary | X¹⁴ | X | X | X | X | X | X | X | X | X | X |
| BSFS, mBSFS-C, | X¹⁴ | X | X | X | X | X | X | X | X | X | X |
| or BITSS | | | | | | | | | | | |
| CGIS | | X | | X | X | X | X | | X | | |
| CGIC | | | | X | X | X | X | | X | | |
| Physician | X | X | X | X | X | X | X | X | X | X | X |
| Assessment of | | | | | | | | | | | |
| Facial | | | | | | | | | | | |
| Physician | X | X | X | X | X | X | X | X | X | X | X |
| assessment of | | | | | | | | | | | |
| Peripheral Edema¹⁵ | | | | | | | | | | | |
| Abdominal | X | X | X | X | X | X | X | X | X | X | X |
| Circumference | | | | | | | | | | | |
| Body Weight with | X¹⁶ | X | | | | | X | | X | | X |
| z Score | | | | | | | | | | | |
| Height with | X¹⁶ | X | | | | | X | | X | | X |
| z Score | | | | | | | | | | | |
| PedsQL Generic | | X | | X | X | X | X | | X | | X |
| Core Scales | | | | | | | | | | | |
| PedsQL GI | | X | | X | X | X | X | | X | | X |
| Symptom Scales | | | | | | | | | | | |
| PedsQL Family | | X | | X | X | X | X | | X | | X |
| Impact Module | | | | | | | | | | | |
| PGIS | | X | | X | X | X | X | | X | | X |
| PGIC | | | | X | X | X | X | | X | | X |
| Hospitalization | X¹⁷ | X | X | X | X | X | X | X | X | X | X |
| Days | | | | | | | | | | | |

Safety

| | | | | | | | | | | | |
|---|---|---|---|---|---|---|---|---|---|---|---|
| Vital signs | X | X | X | X | X | X | X | X | X | X | X |
| Physical | X | X | X | X | X | X | X | X | X | X | X |
| examination | | | | | | | | | | | |
| Adverse events¹⁸ | X | X | X | X | X | X | X | X | X | X | X |

Laboratory testing and biomarkers

| | | | | | | | | | | | |
|---|---|---|---|---|---|---|---|---|---|---|---|
| Blood Chemistry | X | X | X | X | X | X | X | X | X | X | X |
| Panel¹⁹ | | | | | | | | | | | |
| Micronutrients and | | X | | X | | | X | | X | | X |
| Lipid Panel²⁰ | | | | | | | | | | | |
| Blood | | X | | X | | | X | | X | | X |
| Immunoglobulin | | | | | | | | | | | |
| Panel²¹ | | | | | | | | | | | |
| Alpha-1 Antitrypsin | | X | | X | | | X | | | | X |
| (fecal and blood) | | | | | | | | | | | |
| Pregnancy Test²² | X | | | | | | | | | | |
| Urinalysis | X | X | | X | X | X | X | | | | X |
| Hematology | X | X | | X | X | | X | | X | | X |
| Coagulation Panel | X | | | X | | | X | | | | X |
| (APTT/PT) | | | | | | | | | | | |
| Complement Hemolytic | | X | | X | | | X | | X | | X |
| Assay (CH50)²³ | | | | | | | | | | | |
| Total C5²³ | | X | | X | | | X | | X | | X |
| Total Complement | X | | | | | | | | | | |
| C3 and C4 levels | | | | | | | | | | | |
| Thrombosis Biomarkers²⁴ | | X | | X | | | X | | | | X |
| sC5b-9 (plasma); | | X | | X | X | | X | | X | | X |
| C5a (plasma & | | | | | | | | | | | |
| urine)²³ | | | | | | | | | | | |
| Buccal Swab for | | X | | | | | | | | | |
| DNA Isolation | | | | | | | | | | | |
| (optional)²⁵ | | | | | | | | | | | |
| Future biomedical | | X | | | | | X | | X | | X |
| research | | | | | | | | | | | |
| (optional, weight | | | | | | | | | | | |
| >20 kg only) | | | | | | | | | | | |

Drug Concentration and ADA Samples

| | | | | | | | |
|---|---|---|---|---|---|---|---|
| Pozelimab | X | X | X | X | X | X | X |
| concentration | | | | | | | |
| sample^{23, 27} | | | | | | | |
| ADA sample^{23, 27, 28} | X | | | | | | X |

TABLE 4-2
Schedule of Events (Visits 12-25)

| Study procedure | | | | | | | | | | | | | | |
|---|---|---|---|---|---|---|---|---|---|---|---|---|---|---|
| Treatment period | | | | | | | | | | | | EOT | EOS | |
| Visit No | | | | | | | | | | | | | | |
| 12 | 13 | 14 | 15 | 16 | 17 | 18 | 19 | 20 | 21 | 22 | 23 | 24 | 25 | |
| Day | | | | | | | | | | | | | | |
| 99 ± | 113 ± | 127 ± | 141 ± | 155 ± | 169 ± | 253 ± | 337 ± | 421 ± | 505 ± | 589 ± | 673 ± | 729 ± | 813 ± | |
| 3 | 3 | 3 | 3 | 3 | 3 | 3 | 3 | 3 | 3 | 3 | 3 | 3 | 3 | |
| Week | | | | | | | | | | | | | | |
| 14 | 16 | 18 | 20 | 22 | 24 | 36 | 48 | 60 | 72 | 84 | 96 | 104 | 116 | ET |

Treatment

| Admin pozelimab¹⁰ | X¹² | | | | | | | | | | | | | | |
|---|---|---|---|---|---|---|---|---|---|---|---|---|---|---|---|
| Patient diary | X | X | X | X | X | X | X | X | X | X | X | X | X | | |
| Comcomitant meds | X | X | X | X | X | X | X | X | X | X | X | X | X | X | X |
| and interventions | | | | | | | | | | | | | | | |

Efficacy

| | | | | | | | | | | | | | | | |
|---|---|---|---|---|---|---|---|---|---|---|---|---|---|---|---|
| Tanner Staging¹³ | | | | | | X | | X | | X | | | X | | |
| E-Diary | X | X | X | X | X | X | | | | | | | | | |
| BSFS, mBSFS-C, | X | X | X | X | X | X | | | | | | | | | |
| or BITSS | | | | | | | | | | | | | | | |
| CGIS | | X | | | | X | | X | | X | | X | X | | |
| CGIC | | X | | | | X | | | | | | | X | | |
| Physician | | X | | X | | X | X | X | X | X | X | X | X | | |
| Assessment of | | | | | | | | | | | | | | | |
| Facial Edema¹⁵ | | | | | | | | | | | | | | | |
| Physician | | X | | X | | X | X | X | X | X | X | X | X | | |
| Assessment of | | | | | | | | | | | | | | | |
| Peripheral Edema¹⁵ | | | | | | | | | | | | | | | |
| Abdominal | | X | | X | | X | | | | | | | | | |
| Circumference¹⁵ | | | | | | | | | | | | | | | |
| Body Weight with | | X | | X | | X | X | X | X | X | X | X | X | | |
| z Score¹⁶ | | | | | | | | | | | | | | | |
| Height with | | X | | X | | X | X | X | X | X | X | X | X | | |
| z Score¹⁶ | | | | | | | | | | | | | | | |
| PedsQL Generic | | X | | | | X | | | | | | | X | | |
| Core Scales | | | | | | | | | | | | | | | |
| PedsQL GI | | X | | | | X | | X | | X | | X | X | | |
| Symptom Scales | | | | | | | | | | | | | | | |
| PedsQL Family | | X | | | | X | | | | | | | | | |
| Impact Module | | | | | | | | | | | | | | | |
| PGIS | | X | | | | X | | X | | X | | X | X | | |
| PGIC | | X | | | | X | | X | | | | | X | | |
| Exit interview⁹ | | | | | | X | | | | | | | | | X |
| Hospitalization | | X | | X | | X | X | X | X | X | X | X | X | X | X |
| Days | | | | | | | | | | | | | | | |

Safety

| | | | | | | | | | | | | |
|---|---|---|---|---|---|---|---|---|---|---|---|---|
| Vital signs | X | X | X | X | X | X | X | X | X | X | | |
| Physical Exam | X | X | X | X | X | X | X | X | X | X | | |
| Adverse Events | X | X | X | X | X | X | X | X | X | X | X | X |

Laboratory testing and biomarkers

| | | | | | | | | | | | | | | | |
|---|---|---|---|---|---|---|---|---|---|---|---|---|---|---|---|
| Blood Chemistry | X²⁶ | X | X²⁶ | X | X²⁶ | X | X | X | X | X | X | X | X | | X |
| Panel¹⁹ | | | | | | | | | | | | | | | |
| Micronutrients and | | X | | X | | X | X | X | | X | | X | X | | X |
| Lipid Panel²⁰ | | | | | | | | | | | | | | | |
| Blood | | X | | X | | X | X | X | X | X | X | X | X | | X |
| Immunoglobulin | | | | | | | | | | | | | | | |
| Panel | | | | | | | | | | | | | | | |
| Alpha 1- Antitrypsin | | | | | | X | | | | | | | | | |
| (fecal and blood) | | | | | | | | | | | | | | | |
| Pregnancy Test²² | | | | | | X | | | | | | | X | X | X |
| Hematology | | X | | X | | X | X | X | | X | | X | X | | |
| Coagulation Panel | | | | | | X | | X | | | | | X | | |
| (APTT/PT) | | | | | | | | | | | | | | | |
| Complement Hemolytic | | X | | X | | X | X | X | | X | | X | X | | |
| Assay (CH50)²³ | | | | | | | | | | | | | | | |
| Total C5²³ | | X | | X | | X | X | X | | X | | X | X | | |
| Total Complement | X | | | | | | | | | | | | | | |
| C3 and C4 Levels | | | | | | | | | | | | | | | |
| Thrombosis Biomarkers²⁴ | | | | | | X | | X | | | | | X | | |
| sC5b-9 (plasma); | | | | | | X | | X | | X | | | X | | |
| C5a (plasma & | | | | | | | | | | | | | | | |
| urine)²³ | | | | | | | | | | | | | | | |
| Buccal Swab for | | | | | | | | | | | | | | | |
| DNA Isolation | | | | | | | | | | | | | | | |
| (optional)²⁵ | | | | | | | | | | | | | | | |
| Future biomedical | | X | | X | | X | | X | | | | | X | | |
| research | | | | | | | | | | | | | | | |
| (optional, body | | | | | | | | | | | | | | | |
| weight >20 kg only) | | | | | | | | | | | | | | | |

Drug concentration and ADA samples

| | | | | | | | | | | |
|---|---|---|---|---|---|---|---|---|---|---|
| Pozelimab | X | X | X | X | X | X | X | X | X | X |
| concentration | | | | | | | | | | |
| sample^{23, 27} | | | | | | | | | | |
| ADA sample^{23, 27} | | | X | | X | | | X | X | X |

EOT = End of TX analysis
EOS = End of Study
ET—Early term
CGIS = Clinical global impression of severity
CGIC = Clinical global impression of change
PT = prothrombin time
APTT = activated partial thromboplastin time
BITSS = Brussels infant and toddler stool scale
BSFS = Bristol stool form scale
mBSFS-C = modified Bristol Stool Form Scale for Children
ADA = -anti-drug antibody
PedsQL = Pediatric Quality of Life Inventory

1. Treatment period is from first dose at week 0 to last dose at week 103. All visits in the Schedule of Events table are mandatory in-clinic visits, and do not reflect the dosing schedule, which is weekly. Study procedures within each visit may be conducted on different days, within the stated visit window.
2. Including history of CD55 gene mutation analysis and if necessary CD55 protein analysis, confirmed by flow cytometry or western blot, respectively. If this data is unavailable, a blood sample may be collected, as needed, for analysis.
3. Including history of albumin infusions and prior thromboembolic events since birth
4. Including eculizumab administration history.
5. Albumin, total protein, total immunoglobulin data including everything available from the patient's birth onwards.
6. All patients require meningococcal, pneumococcal, and H. influenzae vaccination, either prior to the study or during screening, according to local availability and practice guidelines.
7. Patients should be counseled about Neisseria gonorrhea prevention and regular testing should be advised for at-risk patients, as applicable. A risk factor assessment should be based on local practice or national guidelines. The investigator should make his/her own assessment of risk (and if needed, consultation with other healthcare provider) to determine if the patient is at risk, which would lead to further management on prevention, testing, and treatment of Neisseria gonorrhea. Testing and treatment should be in accordance with local practice/national guidelines. General preventive measures include abstinence and use of a condom. Additional preventive measures should be considered based on local practice or national guidelines.
8. Screening by tuberculin skin test or T-cell interferon-gamma release assay may be performed according to local practice or guidelines at the discretion of the investigator.
9. Patients (and caregivers, as appropriate) will be undergo a concept elicitation interview at screening and an exit interview at the time point of the primary endpoint as part of clinical outcomes evaluation.
10. Meningococcal vaccination is required and daily oral antibiotic prophylaxis is recommended.
11. IV loading dose.
12. Subcutaneous dosing to be administered weekly either at study site or in local community healthcare setting close to patient or at home. Weekly dosing is not noted as visits on this SOE table. The last dose of study drug is administered at week 103.
13. Only for patients between ages 8 to 20 years.
14. Patients to begin completion of e-diary recording bowel movements and consistency at least 7 days prior to the baseline visit.
15. In the presence of facial or peripheral edema or ascites, assessment should be accompanied by clinical photography where available.
16. Including all available historical height and weight data from birth.
17. Collect all available information pertaining to previous hospitalization dates since birth.
18. Including new thromboses and extension of existing thromboses.
19. Total protein and albumin are tested in this panel. Testing will use either adult or small-volume pediatric kits as specified in a manual or kit instruction. If patient receives IV albumin infusion, this panel should be drawn either prior to the infusion or 2 weeks after the infusion.
20. Samples for laboratory testing will be collected at visits according to the Schedule of Events Table.

Hematology, chemistry (except Total C5, CH50 sC5b-9 and C5a), urinalysis, and pregnancy testing samples may be analyzed by a local/central laboratory. Other testing will be done by a central or specialized laboratory as outlined in the sample management plan. Detailed instructions for blood sample collection are in the sample management plan provided to study sites.

### Blood Chemistry

| | | |
|---|---|---|
| Sodium | Total protein, serum albumin | Total bilirubin |
| Potassium | Creatine (eGFR) | Total cholesterol |
| | | ((low-density |
| | | lipoprotein [LDL] |
| | | and high-density |
| | | lipoprotein [HDL])) |
| Chloride | Blood urea nitrogen | Triglycerides |
| | (BUN)/Urea | |
| Calcium | Aspartate aminotransferase | Uric acid |
| Glucose | Alanine aminotransferase | Creatine kinase |
| Albumin | Alkaline phosphatase | |
| magnesium | Lactate dehydrogenase | |

Fasting lipids and glucose should be obtained at the baseline visit and visits at weeks 12 and 24, if possible

### Lipid Panel (Fasting)

Total cholesterol (LDL and HDL)

### Triglycerides

### Blood Immunoglobin Panel

### Total Ig, IgG, IgM, IgA

### Micronutrient Panel

Vitamin B12, folate, iron, iron-binding capacity, ferritin

### Hematology Panel

| | |
|---|---|
| Hemoglobin | Differential: |
| Hematocrit | Neutrophils |
| Red blood cells | Lymphocytes |
| White blood cells | Monocytes |
| Red cell indices | Basophils |
| Platelet count | Eosinophils |
| Reticulocyte count | |

### Coagulation Panel

PT/aPTT (PT/aPTT Prothrombin time/activated partial thromboplastin time)

### Urinalysis

### Glucose

Protein—Note: If protein is ++ or more then reflex to urinary protein creatinine ratio
Blood—Note: if blood is ++ or more then reflex to microscopy

### Other Laboratory Tests

Other laboratory tests may include:
Complement hemolytic assay (CH50)
Alpha-1 antitrypsin
Pregnancy testing: serum human chorionic gonadotropin pregnancy testing, urine pregnancy testing
Sample collection is described separately for drug concentration, ADA, and exploratory biomarkers

### 21. See Blood Immunoglobulin Panel.

22. According to local practice in the study country, pregnancy testing (urinary human chorionic gonadotropin) is mandatory for all females from the age of sexual maturity, or for married females and, at the discretion of the investigator, for non-married females from the age of sexual maturity.
23. Intensity of blood sampling for these analytes will be reduced if necessary to comply with local body weight-specific limitations on blood withdrawal volume. The blood draw schedule in the SOE Table is designed for patients with body weight equal or greater than 20 kg. It is expected that patients below 20 kg in weight will require reduction in blood-draw intensity. A separate blood draw schedule will be provided for patients with body weight between 10 kg and 20 kg in the sample handling manual. For patients with body weight less than 10 kg, an order of priority of blood draws will be provided in the sample handling manual or kit instruction, and samples should be drawn in this order until the volume limit is reached. The chemistry panel will have highest priority followed by full blood count and drug concentration.
24. May include D-dimer, F(1+2).
25. Sample should be collected at baseline visit but may be collected at any time.
26. Kits may be provided locally so that the chemistry panel may be taken locally to the patient without needing a site visit.
27. Drug concentration and ADA samples are to be collected prior to study drug administration. In the event of any SAE or any AESI of anaphylactic reactions or systemic allergic reactions that are related to study drug and require treatment, or severe injection site reaction lasting longer than 24 hours, drug concentration and ADA samples will be collected at or near the onset of the event for any additional analysis.
28. In the event a patient sample is positive in the pozelimab ADA assay at week 12 or the first time point analyzed, the week 4 PK sample may be analyzed in the ADA assay, provided there is sufficient volume.
29. The screening period may be extended to approximately 10 weeks for patients with extenuating circumstances.

### COVID-19

In light of the public health emergency related to COVID-19, the continuity of clinical study conduct and oversight may require implementation of temporary or alternative mechanisms. Examples of such mechanisms may include, but are not limited to, any of the following: phone contact, virtual visits, telemedicine visits, online meetings, non-invasive remote monitoring devices, use of local clinic or laboratory locations, and home visits by skilled staff. Additionally, no waivers to deviate from protocol enrollment criteria due to COVID-19 will be granted. All temporary mechanisms utilized, and deviations from planned study procedures in response to COVID-19 are to be documented as being related to COVID-19 and will remain in effect only for the duration of the public health emergency.

### Results

The patients receiving the pozelimab dosing regimen achieved improvements in albumin, total protein, vitamin B12, platelets, fecal a1AT, edema of face, edema of limbs, some suggestion of improvement in abdominal pain scores and bowel movement frequency; and an early indication of reduction in hospitalization days and a reduction in steroid use.

Albumin and total protein. CHAPLE is characterized by a loss of serum protein, such as albumin, into the gastrointestinal tract resulting in hypoproteinemia, which can be complicated by edema, ascites, pleural and pericardial effusions, and malnutrition. In healthy individuals, loss of protein through the gut epithelium has only a minor role in total protein metabolism. Gastrointestinal (GI) protein loss in CHAPLE can involve up to 60% of the total albumin pool. Patients receiving the pozelimab regimen exhibited more normal levels of serum albumin and total protein suggesting alleviation of GI protein loss. Shortly after initiation of treatment, albumin levels improved (increased to at or above the lower level of normal (LLN)) and remained at or above the LLN at all time points measured (FIG. 28(a)). Monitoring of albumin levels for each patient prior to treatment demonstrated that albumin levels were historically below normal (FIG. 28(b)-(e)). Moreover, shortly after initiation of treatment, total protein levels improved (increased to between the lower level of normal (LLN) and upper level of normal (ULN)) and remained within this normal range at all time points measured (FIG. 29).

Vitamin B12. Malabsorption and deficiency of vitamins, such as B12, have been observed in protein-losing enteropathy. Vitamin B12 levels in patients receiving the pozelimab regimen improved over time. This is possibly due to alleviation of GI malabsorption in CHAPLE patients. These patients did not receive vitamin B12 supplementation. Shortly after initiation of treatment, vitamin B12 levels improved and the elevated levels were maintained at all time points measured (FIG. 30).

Platelets. Excessive complement activation can lead to induction of the coagulation cascade. The loss of GPI-anchored complement inhibitory proteins, such as CD55, can lead to terminal-complement mediated hemolysis with a secondary thrombotic risk. Indeed, CHAPLE patients have an increased risk of thrombosis. Patients receiving the pozelimab regimen benefited by a decrease in platelet counts. Shortly after initiation of treatment, platelet counts decreased and remained at the lower levels at all time points measured (FIG. 31).

Fecal alpha-1-antitrypsin. Alpha-1-antitrypsin (A1A) is resistant to degradation by digestive enzymes and is, therefore, used as an endogenous marker for the presence of blood proteins in the intestinal tract. Patients receiving the pozelimab regimen exhibited decreases in A1A. Shortly after initiation of treatment, fecal alpha-1-antitrypsin concentration decreased in each patient and remained at the lower levels at all time points measured (FIG. 32).

Facial and peripheral edema. CHAPLE is characterized by excessive loss of serum proteins into the gastrointestinal tract. This leads to reduced levels of serum proteins that, if severe, causes loss of fluid from the intravascular space and edema. There was evidence of alleviation of edema in patients receiving the pozelimab regimen. Shortly after initiation of treatment, the severity (grade) of facial and peripheral edema generally decreased in the patients and remained at the lower levels at all time points measured (FIG. 33 and FIG. 34).

Bowel movement frequency. Patients suffering from CHAPLE disease typically have diarrhea and excessive bowel movement frequency. These factors have significant impacts on patient quality of life and can lead to secondary medical conditions such as vitamin or electrolyte imbalances. There was evidence that patients on the pozelimab regimen achieved an improvement in bowel movement frequency. Shortly after initiation of treatment there were early indication of a reduction in the frequency of bowel movements in the patients (FIG. 35).

The present invention, thus, provides methods for:
Increasing serum albumin level, or decreasing loss thereof through the GI tract;
Increasing total serum protein level, or decreasing loss thereof through the GI tract;
Increasing serum vitamin (e.g., vitamin B12), e.g., in the absence of supplementation of such vitamin, or GI absorption thereof;
Decreasing platelet counts or decreasing coagulation cascade activation or decreasing the incidence of thrombotic events (e.g., heart attack, stroke);
Decreasing the loss of alpha-1-antitrypsin through the GI tract;
Treating or preventing edema (e.g., facial or peripheral);
Decreasing the frequency of bowel movements or treating or preventing diarrhea;
Treating or preventing abdominal pain;
Decreasing the use of steroids (e.g., corticosteroids such as cortisone, hydrocortisone or prednisone);
Decreasing the incidence of hospitalization;
   in a patient with a C5-associated disease, such as CHAPLE disease, by administering, to the patient,
   (i) one or more doses of about 30 mg/kg of antagonist antigen-binding protein that binds specifically to C5 (e.g., pozelimab) intravenously (IV); then
   (ii) one or more doses (e.g., weekly doses) of about 800 mg of the antagonist antigen-binding protein that binds specifically to C5, subcutaneously (SC);
   or
   (i) one or more doses of about 30 mg/kg of antagonist antigen-binding protein that binds specifically to C5 (e.g., pozelimab) intravenously (IV); then
   (ii) one or more doses (e.g., weekly doses) of the antagonist antigen-binding protein that binds specifically to C5, subcutaneously (SC), according to the following:
for body weight (BW)<10 kg: 125 mg;
for BW ≥10 kg and <20 kg: 200 mg;
for BW ≥20 kg and <40 kg: 350 mg;
for BW ≥40 kg and <60 kg: 500 mg; and
for BW ≥60 kg: 800 mg.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A method for administering an antagonist antigen-binding protein that binds specifically to C5 or a pharmaceutical formulation thereof, to a subject suffering from a C5-associated disease, comprising introducing, into the body of the subject, one or more doses of about 30 mg/kg of the antigen-binding protein intravenously; and, optionally, one or more doses of the antigen-binding protein or a pharmaceutical formulation thereof subcutaneously.

2. A method for administering an antagonist antigen-binding protein that binds specifically to C5 or a pharmaceutical formulation thereof, to a subject, comprising introducing, into the body of the subject,
(i) one or more doses of about 30 mg/kg of the antigen-binding protein intravenously (IV); then
(ii) one or more doses of about 800 mg of the antigen-binding protein, subcutaneously (SC);
or,
(i) one or more doses of about 30 mg/kg of the antigen-binding protein intravenously (IV); then
(ii) one or more doses of the antigen-binding protein, subcutaneously (SC), according to the following:
for body weight (BW)<10 kg: 125 mg;
for BW ≥10 kg and <20 kg: 200 mg;
for BW ≥20 kg and <40 kg: 350 mg;
for BW ≥40 kg and <60 kg: 500 mg; and
for BW ≥60 kg: 800 mg.

3. The method of claim 1, wherein the subject is a human.

4. The method of claim 1, wherein the subcutaneous doses are administered once a week.

5. The method of claim 1, wherein only a single intravenous dose is administered.

6. The method of claim 4, wherein said weekly doses are administered about 7 days, 7 days (±1 day), 7 days (±2 days) or 7 days (±3 days) after the immediately preceding dose.

7. The method of claim 1, wherein the subject suffers from a C5-associated disease.

8. The method of claim 1, wherein the subcutaneous doses are administered to the subject with a pre-filled syringe.

9. A method for treating or preventing CD55-deficient protein-losing enteropathy in a subject in need thereof by administering, to the subject, a therapeutically effective amount of antagonist antigen-binding protein that binds specifically to C5 which comprises one or more selected from the group consisting of:
(1) a heavy chain variable region (HCVR) that comprises the amino acid sequence set forth in SEQ ID NO: 2 or HCDR1, HCDR2 and HCDR3 thereof, and a light chain variable region (LCVR) that comprises the amino acid sequence set forth in SEQ ID NO: 10 or LCDR1, LCDR2 and LCDR3 thereof;
(2) a HCVR that comprises the amino acid sequence set forth in SEQ ID NO: 18 or HCDR1, HCDR2 and HCDR3 thereof, and an LCVR that comprises the amino acid sequence set forth in SEQ ID NO: 26 or LCDR1, LCDR2 and LCDR3 thereof;
(3) a HCVR that comprises the amino acid sequence set forth in SEQ ID NO: 34 or HCDR1, HCDR2 and HCDR3 thereof, and an LCVR that comprises the amino acid sequence set forth in SEQ ID NO: 42 or LCDR1, LCDR2 and LCDR3 thereof;
(4) a HCVR that comprises the amino acid sequence set forth in SEQ ID NO: 50 or HCDR1, HCDR2 and HCDR3 thereof, and an LCVR that comprises the amino acid sequence set forth in SEQ ID NO: 58 or LCDR1, LCDR2 and LCDR3 thereof;
(5) a HCVR that comprises the amino acid sequence set forth in SEQ ID NO: 66 or HCDR1, HCDR2 and HCDR3 thereof, and an LCVR that comprises the amino acid sequence set forth in SEQ ID NO: 74 or LCDR1, LCDR2 and LCDR3 thereof;
(6) a HCVR that comprises the amino acid sequence set forth in SEQ ID NO: 82 or HCDR1, HCDR2 and HCDR3 thereof, and an LCVR that comprises the amino acid sequence set forth in SEQ ID NO: 90 or LCDR1, LCDR2 and LCDR3 thereof;
(7) a HCVR that comprises the amino acid sequence set forth in SEQ ID NO: 98 or HCDR1, HCDR2 and HCDR3 thereof, and an LCVR that comprises the amino acid sequence set forth in SEQ ID NO: 106 or LCDR1, LCDR2 and LCDR3 thereof;
(8) a HCVR that comprises the amino acid sequence set forth in SEQ ID NO: 98 or HCDR1, HCDR2 and HCDR3 thereof, and an LCVR that comprises the amino acid sequence set forth in SEQ ID NO: 114 or LCDR1, LCDR2 and LCDR3 thereof;
(9) a HCVR that comprises the amino acid sequence set forth in SEQ ID NO: 122 or HCDR1, HCDR2 and HCDR3 thereof, and an LCVR that comprises the amino acid sequence set forth in SEQ ID NO: 106 or LCDR1, LCDR2 and LCDR3 thereof;
(10) a HCVR that comprises the amino acid sequence set forth in SEQ ID NO: 98 or HCDR1, HCDR2 and HCDR3 thereof, and an LCVR that comprises the amino acid sequence set forth in SEQ ID NO: 130 or LCDR1, LCDR2 and LCDR3 thereof;
(11) a HCVR that comprises the amino acid sequence set forth in SEQ ID NO: 138 or HCDR1, HCDR2 and HCDR3 thereof, and an LCVR that comprises the amino acid sequence set forth in SEQ ID NO: 106 or LCDR1, LCDR2 and LCDR3 thereof;
(12) a HCVR that comprises the amino acid sequence set forth in SEQ ID NO: 146 or HCDR1, HCDR2 and HCDR3 thereof, and an LCVR that comprises the amino acid sequence set forth in SEQ ID NO: 106 or LCDR1, LCDR2 and LCDR3 thereof;
(13) a HCVR that comprises the amino acid sequence set forth in SEQ ID NO: 122 or HCDR1, HCDR2 and HCDR3 thereof, and an LCVR that comprises the amino acid sequence set forth in SEQ ID NO: 130 or LCDR1, LCDR2 and LCDR3 thereof;
(14) a HCVR that comprises the amino acid sequence set forth in SEQ ID NO: 146 or HCDR1, HCDR2 and HCDR3 thereof, and an LCVR that comprises the amino acid sequence set forth in SEQ ID NO: 114 or LCDR1, LCDR2 and LCDR3 thereof;
(15) a HCVR that comprises the amino acid sequence set forth in SEQ ID NO: 146 or HCDR1, HCDR2 and HCDR3 thereof, and an LCVR that comprises the amino acid sequence set forth in SEQ ID NO: 130 or LCDR1, LCDR2 and LCDR3 thereof;
(16) a HCVR that comprises the amino acid sequence set forth in SEQ ID NO: 138 or HCDR1, HCDR2 and HCDR3 thereof, and an LCVR that comprises the amino acid sequence set forth in SEQ ID NO: 130 or LCDR1, LCDR2 and LCDR3 thereof;
(17) a HCVR that comprises the amino acid sequence set forth in SEQ ID NO: 154 or HCDR1, HCDR2 and HCDR3 thereof, and an LCVR that comprises the amino acid sequence set forth in SEQ ID NO: 162 or LCDR1, LCDR2 and LCDR3 thereof;
(18) a HCVR that comprises the amino acid sequence set forth in SEQ ID NO: 170 or HCDR1, HCDR2 and HCDR3 thereof, and an LCVR that comprises the amino acid sequence set forth in SEQ ID NO: 178 or LCDR1, LCDR2 and LCDR3 thereof;
(19) a HCVR that comprises the amino acid sequence set forth in SEQ ID NO: 186 or HCDR1, HCDR2 and HCDR3 thereof, and an LCVR that comprises the amino acid sequence set forth in SEQ ID NO: 194 or LCDR1, LCDR2 and LCDR3 thereof;
(20) a HCVR that comprises the amino acid sequence set forth in SEQ ID NO: 202 or HCDR1, HCDR2 and HCDR3 thereof, and an LCVR that comprises the amino acid sequence set forth in SEQ ID NO: 210 or LCDR1, LCDR2 and LCDR3 thereof;
(21) a HCVR that comprises the amino acid sequence set forth in SEQ ID NO: 218 or HCDR1, HCDR2 and HCDR3 thereof, and an LCVR that comprises the amino acid sequence set forth in SEQ ID NO: 226 or LCDR1, LCDR2 and LCDR3 thereof;
(22) a HCVR that comprises the amino acid sequence set forth in SEQ ID NO: 234 or HCDR1, HCDR2 and HCDR3 thereof, and an LCVR that comprises the amino acid sequence set forth in SEQ ID NO: 242 or LCDR1, LCDR2 and LCDR3 thereof;
(23) a HCVR that comprises the amino acid sequence set forth in SEQ ID NO: 250 or HCDR1, HCDR2 and HCDR3 thereof, and an LCVR that comprises the amino acid sequence set forth in SEQ ID NO: 258 or LCDR1, LCDR2 and LCDR3 thereof;
(24) a HCVR that comprises the amino acid sequence set forth in SEQ ID NO: 266 or HCDR1, HCDR2 and HCDR3 thereof, and an LCVR that comprises the amino acid sequence set forth in SEQ ID NO: 258 or LCDR1, LCDR2 and LCDR3 thereof;
(25) a HCVR that comprises the amino acid sequence set forth in SEQ ID NO: 274 or HCDR1, HCDR2 and HCDR3 thereof, and an LCVR that comprises the amino acid sequence set forth in SEQ ID NO: 282 or LCDR1, LCDR2 and LCDR3 thereof;
(26) a HCVR that comprises the amino acid sequence set forth in SEQ ID NO: 290 or HCDR1, HCDR2 and HCDR3 thereof, and an LCVR that comprises the amino acid sequence set forth in SEQ ID NO: 298 or LCDR1, LCDR2 and LCDR3 thereof;
(27) a HCVR that comprises the amino acid sequence set forth in SEQ ID NO: 306 or HCDR1, HCDR2 and HCDR3 thereof, and an LCVR that comprises the amino acid sequence set forth in SEQ ID NO: 314 or LCDR1, LCDR2 and LCDR3 thereof;
(28) a HCVR that comprises the amino acid sequence set forth in SEQ ID NO: 322 or HCDR1, HCDR2 and HCDR3 thereof, and an LCVR that comprises the amino acid sequence set forth in SEQ ID NO: 330 or LCDR1, LCDR2 and LCDR3 thereof;
and,
(29) a HCVR that comprises the amino acid sequence set forth in SEQ ID NO: 338 or HCDR1, HCDR2 and HCDR3 thereof, and an LCVR that comprises the amino acid sequence set forth in SEQ ID NO: 346 or LCDR1, LCDR2 and LCDR3 thereof.

10. A method for treating or preventing a C5-associated disease or reducing C5 complement activity, in a subject, comprising administering an antagonist antigen-binding protein that binds specifically to C5, to the subject, by a method according to claim 1.

11. The method of claim 10 wherein the C5 complement activity is reduced by about 95-100% as measured by CH50 assay of complement-mediated sheep red blood cell lysis.

12. The method of claim 10, wherein the C5-associated disease is paroxysmal nocturnal hemoglobinuria (PNH).

13. The method of claim 10, wherein the C5-associated disease is CD55-deficient protein-losing enteropathy (CHAPLE disease).

14. The method of claim 10, wherein the C5-associated disease is:
adult respiratory distress syndrome; age-related macular degeneration (AMD); allergy; alport's syndrome; alzheimer's disease; amyotrophic lateral sclerosis (ALS); antiphospholipid syndrome (APS); asthma; atherosclerosis; atypical hemolytic uremic syndrome (aHUS); an autoimmune disease; autoimmune hemolytic anemia (AIHA); balloon angioplasty; bronchoconstriction; bullous pemphigoid; burns; C3 glomerulopathy; capillary leak syndrome; a cardiovascular disorder; catastrophic antiphospholipid syndrome (CAPS); a cerebrovascular disorder; CHAPLE disease (CD55 deficiency with hyperactivation of complement, angiopathic thrombosis, and protein-losing enteropathy); a chemical injury; chronic obstructive pulmonary disease (COPD); cold agglutinin disease (CAD); corneal and/or retinal tissue; Crohn's disease; Degos disease; dense deposit disease (DDD); dermatomyositis; diabetes; diabetic angiopathy; diabetic macular edema (DME); diabetic nephropathy; diabetic retinopathy; dilated cardiomyopathy; disorder of inappropriate or undesirable complement activation; dyspnea; eclampsia; emphysema; epidermolysis bullosa; epilepsy; fibrogenic dust disease; frostbite; geographic atrophy (GA); glomerulonephritis; glomerulopathy; Goodpasture's Syndrome; Graves' disease; Guillain-Barre Syndrome; Hashimoto's thyroiditis; hemodialysis complications; hemolysis-elevated liver enzymes- and low platelets (HELLP) syndrome; hemolytic anemia; hemoptysis; Henoch-Schonlein purpura nephritis; hereditary angioedema; hyperacute allograft rejection; hypersensitivity pneumonitis; idiopathic thrombocytopenic purpura (ITP); IgA nephropathy; an immune complex disorder; immune complex vasculitis; immune complex-associated inflammation; an infectious disease; inflammation caused by an autoimmune disease; an inflammatory disorder; inherited CD59 deficiency; injury due to inert dusts and/or minerals; interleukin-2 induced toxicity during IL-2 therapy; ischemia-reperfusion injury; Kawasaki's disease; a lung disease or disorder; lupus nephritis; membrane proliferative glomerulonephritis; membrano-proliferative nephritis; mesenteric artery reperfusion after aortic reconstruction; mesenteric/enteric vascular disorder; multifocal motor neuropathy (MMN); multiple sclerosis; myasthenia gravis; myocardial infarction; myocarditis; neurological disorder; neuromyelitis optica; obesity; ocular angiogenesis; ocular neovascularization affecting choroidal; organic dust disease; parasitic disease; Parkinson's disease; paroxysmal nocturnal hemoglobinuria (PNH); pauci-immune vasculitis; pemphigus; percutaneous transluminal coronary angioplasty (PTCA); peripheral vascular disorder; pneumonia; post-ischemic reperfusion condition; post-pump syndrome in cardiopulmonary bypass; post-pump syndrome in renal bypass; pre-eclampsia; progressive kidney failure; proliferative nephritis; proteinuric kidney disease; psoriasis; pulmonary embolism; pulmonary fibrosis; pulmonary infarction; pulmonary vasculitis; recurrent fetal loss; a renal disorder; renal ischemia; renal ischemia-reperfusion injury; a renovascular disorder; restenosis following stent placement; rheumatoid arthritis (RA); rotational atherectomy; schizophrenia; sepsis; septic shock; SLE nephritis; smoke injury; spinal cord injury; spontaneous fetal loss; stroke; systemic inflammatory response to sepsis; systemic lupus erythematosus (SLE); systemic lupus erythematosus-associated vasculitis; Takayasu's disease; thermal injury; thrombotic thrombocytopenic purpura (TTP); traumatic brain injury; type I diabetes; typical hemolytic uremic syndrome (tHUS); uveitis; vasculitis; vasculitis associated with rheumatoid arthritis; venous gas embolus (VGE); and/or; xenograft rejection.

15. A method for maintaining a concentration of at least about 100 mg/L of antagonist antigen-binding protein that binds specifically to C5 in the serum of a subject and/or for maintaining at least 80% suppression of hemolysis in the serum of a subject comprising administering the antagonist antigen-binding protein that binds specifically to C5, to the subject, by the method of claim 1.

16. The method of claim 15 wherein the subject suffers from a C5-associated disease.

17. The method of claim 15, wherein hemolysis is as measured in vitro in a CH50 and/or AH50 assay.

18. A method for:
normalizing and/or increasing serum albumin or decreasing loss thereof through the gastrointestinal tract;
increasing total serum protein level, or decreasing loss thereof through the gastrointestinal tract;
increasing serum vitamin B12 or gastrointestinal absorption thereof;
decreasing platelet counts or decreasing coagulation cascade activation or decreasing the incidence of thrombotic events;
decreasing the loss of alpha-1-antitrypsin through the gastrointestinal tract;
treating or preventing facial and/or peripheral edema;
decreasing the frequency of bowel movements;
treating or preventing diarrhea;
treating or preventing abdominal pain;
decreasing the use of corticosteroids; and/or
decreasing the incidence of hospitalization;
or,
reducing therapeutic interventions,
in a subject suffering from CD55-deficient protein-losing enteropathy, comprising administering the antagonist antigen-binding protein that binds specifically to C5 to the subject by the method of claim 1;
wherein the therapeutic intervention is one or more selected from the group consisting of:
(i) administration of a corticosteroid;
(ii) administration of an immunoglobulin;
(iii) administration of albumin;
(iv) administration of an anti-tumor necrosis factor alpha therapeutic agent;
(v) administration of an immunomodulator;
(vi) administration of a micronutrient;
(vii) administration of enteral or parenteral supplementation;
(viii) administration of an anti-coagulant;
(ix) administration of an antibiotic; and
(x) administration of an anti-platelet agent.

19. The method of claim 18 for increasing serum albumin by at least 1 g/dL and/or for normalizing serum albumin to about 3.5 to about 5.5 g/dL.

20. A method for reducing serum lactate dehydrogenase (LDH) levels, intravascular hemolysis and/or the need for transfusions of red blood cells in a subject suffering from paroxysmal nocturnal hemoglobinuria (PNH) comprising administering the antagonist antigen-binding protein that binds specifically to C5 to the subject by the method of claim 1.

21. The method of claim 1, wherein:
(i) the subject has a serum lactate dehydrogenase (LDH) level ≥2× upper limit of normal (ULN);
(ii) the subject has PNH granulocytes (polymorphonuclear [PMN])) of >10%;
(iii) the subject has hypoalbuminemia of less than or equal to 3.2 g/dL;
(iv) the subject suffers from diarrhea;
(v) the subject suffers from vomiting;
(vi) the subject suffers from abdominal pain;
(vii) the subject suffers from peripheral or facial edema;
(viii) the subject suffers from an episode of infection with concomitant, hypogammaglobulinemia, or a thromboembolic event;
(ix) the subject suffers from fatigue;
(x) the subject suffers from hemoglobinuria;
(xi) the subject suffers from shortness of breath (dyspnea);
(xii) the subject suffers from anemia;
(xiii) the subject suffers from a history of a major adverse vascular event;
(xiv) the subject suffers from dysphagia; and/or
(xv) the subject suffers from erectile dysfunction.

22. The method of claim 1, wherein
(i) the subject has a biallelic loss-of-function mutation in CD55;
(ii) the subject has a biallelic loss-of-function mutation in CD55 which is a frame shift mutation; missense mutation, splice site mutation or nonsense mutation;
(iii) the subject has hypoalbuminemia of less than or equal to 3.2 g/dL serum albumin;
(iv) the subject suffers from diarrhea;
(v) the subject suffers from vomiting;
(vi) the subject suffers from abdominal pain;
(vii) the subject suffers from peripheral or facial edema;
(viii) the subject suffers from an episode of infection with concomitant hypogammaglobulinemia; and/or
(ix) the subject suffers from a thrombotic event.

23. The method of claim 1, wherein the antagonist antigen-binding protein that binds specifically to C5 is an antibody or antigen-binding fragment thereof.

24. The method of claim 23, wherein the antagonist antigen-binding protein that binds specifically to C5 is REGN3918 (pozelimab).

25. The method of claim 1, wherein the subject has previously received tesidolumab, eculizumab and/or ravulizumab.

26. The method of claim 23, wherein the antagonist antigen-binding protein that binds specifically to C5 comprises a:
(1) a heavy chain variable region (HCVR) that comprises the amino acid sequence set forth in SEQ ID NO: 2 or HCDR1, HCDR2 and HCDR3 thereof, and a light chain variable region (LCVR) that comprises the amino acid sequence set forth in SEQ ID NO: 10 or LCDR1, LCDR2 and LCDR3 thereof;
(2) a HCVR that comprises the amino acid sequence set forth in SEQ ID NO: 18 or HCDR1, HCDR2 and HCDR3 thereof, and an LCVR that comprises the amino acid sequence set forth in SEQ ID NO: 26 or LCDR1, LCDR2 and LCDR3 thereof;
(3) a HCVR that comprises the amino acid sequence set forth in SEQ ID NO: 34 or HCDR1, HCDR2 and HCDR3 thereof, and an LCVR that comprises the amino acid sequence set forth in SEQ ID NO: 42 or LCDR1, LCDR2 and LCDR3 thereof;
(4) a HCVR that comprises the amino acid sequence set forth in SEQ ID NO: 50 or HCDR1, HCDR2 and HCDR3 thereof, and an LCVR that comprises the amino acid sequence set forth in SEQ ID NO: 58 or LCDR1, LCDR2 and LCDR3 thereof;
(5) a HCVR that comprises the amino acid sequence set forth in SEQ ID NO: 66 or HCDR1, HCDR2 and HCDR3 thereof, and an LCVR that comprises the amino acid sequence set forth in SEQ ID NO: 74 or LCDR1, LCDR2 and LCDR3 thereof;
(6) a HCVR that comprises the amino acid sequence set forth in SEQ ID NO: 82 or HCDR1, HCDR2 and HCDR3 thereof, and an LCVR that comprises the amino acid sequence set forth in SEQ ID NO: 90 or LCDR1, LCDR2 and LCDR3 thereof;
(7) a HCVR that comprises the amino acid sequence set forth in SEQ ID NO: 98 or HCDR1, HCDR2 and HCDR3 thereof, and an LCVR that comprises the amino acid sequence set forth in SEQ ID NO: 106 or LCDR1, LCDR2 and LCDR3 thereof;
(8) a HCVR that comprises the amino acid sequence set forth in SEQ ID NO: 98 or HCDR1, HCDR2 and HCDR3 thereof, and an LCVR that comprises the amino acid sequence set forth in SEQ ID NO: 114 or LCDR1, LCDR2 and LCDR3 thereof;
(9) a HCVR that comprises the amino acid sequence set forth in SEQ ID NO: 122 or HCDR1, HCDR2 and HCDR3 thereof, and an LCVR that comprises the amino acid sequence set forth in SEQ ID NO: 106 or LCDR1, LCDR2 and LCDR3 thereof;
(10) a HCVR that comprises the amino acid sequence set forth in SEQ ID NO: 98 or HCDR1, HCDR2 and HCDR3 thereof, and an LCVR that comprises the amino acid sequence set forth in SEQ ID NO: 130 or LCDR1, LCDR2 and LCDR3 thereof;
(11) a HCVR that comprises the amino acid sequence set forth in SEQ ID NO: 138 or HCDR1, HCDR2 and HCDR3 thereof, and an LCVR that comprises the amino acid sequence set forth in SEQ ID NO: 106 or LCDR1, LCDR2 and LCDR3 thereof;
(12) a HCVR that comprises the amino acid sequence set forth in SEQ ID NO: 146 or HCDR1, HCDR2 and HCDR3 thereof, and an LCVR that comprises the amino acid sequence set forth in SEQ ID NO: 106 or LCDR1, LCDR2 and LCDR3 thereof;
(13) a HCVR that comprises the amino acid sequence set forth in SEQ ID NO: 122 or HCDR1, HCDR2 and HCDR3 thereof, and an LCVR that comprises the amino acid sequence set forth in SEQ ID NO: 130 or LCDR1, LCDR2 and LCDR3 thereof;
(14) a HCVR that comprises the amino acid sequence set forth in SEQ ID NO: 146 or HCDR1, HCDR2 and HCDR3 thereof, and an LCVR that comprises the amino acid sequence set forth in SEQ ID NO: 114 or LCDR1, LCDR2 and LCDR3 thereof;
(15) a HCVR that comprises the amino acid sequence set forth in SEQ ID NO: 146 or HCDR1, HCDR2 and HCDR3 thereof, and an LCVR that comprises the amino acid sequence set forth in SEQ ID NO: 130 or LCDR1, LCDR2 and LCDR3 thereof;
(16) a HCVR that comprises the amino acid sequence set forth in SEQ ID NO: 138 or HCDR1, HCDR2 and HCDR3 thereof, and an LCVR that comprises the amino acid sequence set forth in SEQ ID NO: 130 or LCDR1, LCDR2 and LCDR3 thereof;
(17) a HCVR that comprises the amino acid sequence set forth in SEQ ID NO: 154 or HCDR1, HCDR2 and HCDR3 thereof, and an LCVR that comprises the amino acid sequence set forth in SEQ ID NO: 162 or LCDR1, LCDR2 and LCDR3 thereof;
(18) a HCVR that comprises the amino acid sequence set forth in SEQ ID NO: 170 or HCDR1, HCDR2 and HCDR3 thereof, and an LCVR that comprises the amino acid sequence set forth in SEQ ID NO: 178 or LCDR1, LCDR2 and LCDR3 thereof;
(19) a HCVR that comprises the amino acid sequence set forth in SEQ ID NO: 186 or HCDR1, HCDR2 and HCDR3 thereof, and an LCVR that comprises the amino acid sequence set forth in SEQ ID NO: 194 or LCDR1, LCDR2 and LCDR3 thereof;
(20) a HCVR that comprises the amino acid sequence set forth in SEQ ID NO: 202 or HCDR1, HCDR2 and HCDR3 thereof, and an LCVR that comprises the amino acid sequence set forth in SEQ ID NO: 210 or LCDR1, LCDR2 and LCDR3 thereof;
(21) a HCVR that comprises the amino acid sequence set forth in SEQ ID NO: 218 or HCDR1, HCDR2 and HCDR3 thereof, and an LCVR that comprises the amino acid sequence set forth in SEQ ID NO: 226 or LCDR1, LCDR2 and LCDR3 thereof;
(22) a HCVR that comprises the amino acid sequence set forth in SEQ ID NO: 234 or HCDR1, HCDR2 and HCDR3 thereof, and an LCVR that comprises the amino acid sequence set forth in SEQ ID NO: 242 or LCDR1, LCDR2 and LCDR3 thereof;
(23) a HCVR that comprises the amino acid sequence set forth in SEQ ID NO: 250 or HCDR1, HCDR2 and HCDR3 thereof, and an LCVR that comprises the amino acid sequence set forth in SEQ ID NO: 258 or LCDR1, LCDR2 and LCDR3 thereof;
(24) a HCVR that comprises the amino acid sequence set forth in SEQ ID NO: 266 or HCDR1, HCDR2 and HCDR3 thereof, and an LCVR that comprises the amino acid sequence set forth in SEQ ID NO: 258 or LCDR1, LCDR2 and LCDR3 thereof;
(25) a HCVR that comprises the amino acid sequence set forth in SEQ ID NO: 274 or HCDR1, HCDR2 and HCDR3 thereof, and an LCVR that comprises the amino acid sequence set forth in SEQ ID NO: 282 or LCDR1, LCDR2 and LCDR3 thereof;
(26) a HCVR that comprises the amino acid sequence set forth in SEQ ID NO: 290 or HCDR1, HCDR2 and HCDR3 thereof, and an LCVR that comprises the amino acid sequence set forth in SEQ ID NO: 298 or LCDR1, LCDR2 and LCDR3 thereof;
(27) a HCVR that comprises the amino acid sequence set forth in SEQ ID NO: 306 or HCDR1, HCDR2 and HCDR3 thereof, and an LCVR that comprises the amino acid sequence set forth in SEQ ID NO: 314 or LCDR1, LCDR2 and LCDR3 thereof;
(28) a HCVR that comprises the amino acid sequence set forth in SEQ ID NO: 322 or HCDR1, HCDR2 and HCDR3 thereof, and an LCVR that comprises the amino acid sequence set forth in SEQ ID NO: 330 or LCDR1, LCDR2 and LCDR3 thereof;
and/or,
(29) a HCVR that comprises the amino acid sequence set forth in SEQ ID NO: 338 or HCDR1, HCDR2 and HCDR3 thereof, and an LCVR that comprises the amino acid sequence set forth in SEQ ID NO: 346 or LCDR1, LCDR2 and LCDR3 thereof;
or
competes for binding to C5 with an antigen-binding protein selected from the group consisting of (1)-(29);
or
binds to the same epitope on C5 as an antigen-binding protein selected from the group consisting of (1)-(29).

27. The method of claim 23, wherein the antagonist antigen-binding protein that binds specifically to C5 is a monoclonal antibody comprising an immunoglobulin heavy chain comprising the amino acid sequence:
QVQLQESGPGLVKPSETLSLTCTVSGDSVSSS
YWTWIRQPPGKGLEWIGYIYYSGSSNYNPSLK
SRATISVDTSKNQFSLKLSSVTAADTAVYYCA
REGNVDTTMIFDYWGQGTLVTVSSASTKGPSV
FPLAPCSRSTSESTAALGCLVKDYFPEPVTVS
WNSGALTSGVHTFPAVLQSSGLYSLSSVVTVP
SSSLGTKTYTCNVDHKPSNTKVDKRVESKYGP
PCPPCPAPEFLGGPSVFLFPPKPKDTLMISRT
PEVTCVVVDVSQEDPEVQFNWYVDGVEVHNAK
TKPREEQFNSTYRVVSVLTVLHQDWLNGKEYK
CKVSNKGLPSSIEKTISKAKGQPREPQVYTLP
PSQEEMTKNQVSLTCLVKGFYPSDIAVEWESN
GQPENNYKTTPPVLDSDGSFFLYSRLTVDKSR
WQEGNVFSCSVMHEALHNHYTQKSLSLSLGK
(SEQ ID NO: 368) or a variable region thereof or HCDR1, HCDR2 and HCDR3 thereof; and,
an immunoglobulin light chain comprising the amino acid sequence:
AIQMTQSPSSLSASVGDRVTITCRASQGIRND
LGWYQQKPGKAPKLLIYAASSLQSGVPSRFAG
RGSGTDFTLTISSLQPEDFATYYCLQDFNYPW
TFGQGTKVEIKRTVAAPSVFIFPPSDEQLKSG
TASVVCLLNNFYPREAKVQWKVDNALQSGNSQ
ESVTEQDSKDSTYSLSSTLTLSKADYEKHKVY
ACEVTHQGLSSPVTKSFNRGEC
(SEQ ID NO: 369) or a variable region thereof or LCDR1, LCDR2 and LCDR3 thereof.

28. The method of claim 1, wherein the antagonist antigen-binding protein that binds specifically to C5 is administered or introduced in association with a further therapeutic agent.

29. The method of claim 28 wherein the further therapeutic agent is acetaminophen, an albumin infusion, ancrod, an angiotensin-converting enzyme inhibitor, an antibiotic, an oral antibiotic, a further antibody, an anti-CD20 agent, an anti-coagulant, an anti-fungal agent, an antihypertensive, an anti-inflammatory drug, antiplasmin-a1, an anti-seizure agent, anti-thrombotic agent, an anti-TNFalpha agent, an anti-viral agent, argatroban, aspirin, a biological therapeutic agent, bivalirudin, a C3 inhibitor, a corticosteroid, cyclosporine A, dabigatran, defibrotide, E-aminocaproic acid, enteral feeding, erythromycin, erythropoietin, a fibrinolytic agent, folic acid, fondaparinux, heparin, hormone replacement therapy, ibuprofen, idraparinux, an immunosuppressive drug, infliximab, an inhibitor of hydroxymethylglutaryl CoA reductase, an iron supplement, lepirudin, lipid-lowering agent, magnesium sulfate, a Meningococcal vaccine, methotrexate, a non-steroidal anti-inflammatory drug (NSAID), an oligonucleotide, paracetamol, parenteral feeding, penicillin, phenindione, a pregnancy contraceptive drug, prostacyclin, rituximab, a thrombin inhibitor, a vaccine, vincristine, a vitamin and/or warfarin.

30. The method of claim 28 wherein the further therapeutic agent is an oligonucleotide which is:
a DNA oligonucleotide,
an RNA oligonucleotide,
a single stranded DNA oligonucleotide,
a single stranded RNA oligonucleotide,
a double stranded DNA oligonucleotide, or
a double stranded RNA oligonucleotide;
optionally, wherein the oligonucleotide is conjugated to a sugar.
